(19) United States
(12) Patent Application Publication
(10) Pub. No.: US 2026/0199325 A1
(43) Pub. Date: Jul. 16, 2026

(54) NOVEL SUBSTITUTED PYRAZINE-CARBOXAMIDE-IMIDAZOPYRIDINE DERIVATIVES

(71) Applicant: Boehringer Ingelheim International GmbH, Ingelheim am Rhein (DE)

(72) Inventors: Roland PFAU, Mittlebiberach (DE); Georg DAHMANN, Biberach (DE); Kai GERLACH, Mittlebiberach (DE); Riccardo GIOVANNINI, Biberach an der Riss (DE); Johann Faustus DU HOFFMANN, Biberach an der Riss (DE); Christoph HOHN, Freiburg (DE); Stefan JUST, Biberach an der Riss (DE); Heiko SOMMER, Biberach an der Riss (DE); Christian SPECKER, Hochdorf (DE)

(21) Appl. No.: 19/135,874

(22) PCT Filed: Dec. 8, 2023
(86) PCT No.: PCT/EP2023/084851

§ 371 Date: Jun. 5, 2025

(30) Foreign Application Priority Data
Dec. 8, 2022 (EP) 22212299.6

(51) Int. Cl. A61K 31/497; C07D 401/04

(52) U.S. Cl. CPC A61K 31/497; C07D 401/04

(57) ABSTRACT

The present invention relates to compounds of formula(I), a process for their manufacture, pharmaceutical compositions containing them and their use in therapy, particularly in the treatment and/or prevention of conditions having an association with the function of metabotropic glutamate receptor subtype 4 (mGluR4). A, Xa, Xb, Xc, Xd, A, R¹ and R⁶ have meanings given in the description.

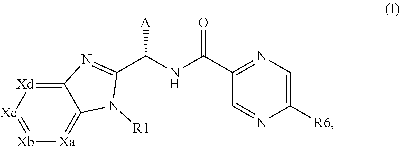

### FIELD OF THE INVENTION

The present invention relates to substituted pyrazine-carboxamide derivatives, pharmaceutical compositions containing them and their use in therapy, particularly in the treatment and/or prevention of neuronal and non-neuronal conditions having an association with mGluR4 function.

### BACKGROUND OF THE INVENTION

L-glutamate (here referred to as glutamate) is among the most abundant excitatory neurotransmitters within the vertebrate brain. Malfunction of the brain glutamate system often leads to neurological or psychiatric disorders. Therefore, modulation of the glutamatergic system is considered as attractive therapeutic direction.

Glutamate acts via different types of glutamate receptor, which are located on the cell surface. Glutamate receptors include AMPA receptors, kainate receptors, NMDA receptors, and metabotropic glutamate receptors. The metabotropic glutamate receptors (mGluR) exert their action via coupling to G proteins and activation of second messenger systems.

The mGluR subtypes are classified into three groups (distinction by sequence homology, pharmacology, second messenger system) with Group III being the largest group (mGluR4, mGluR6, mGluR7, mGluR8) [Conn and Pin, Annu Rev Pharmacol Toxicol, 1997, 37: 205-237]. Group III mGlu receptors share mainly presynaptic expression (Schoepp, Pharmacol Exp Ther, 2001, 299: 12-20) where they modulate glutamatergic as well as GABAergic transmission. Activation of Group III receptors (including mGluR4), reduces transmitter release due to its activation of the Gαi/o which leads to attenuated adenylate cyclase activity.

The mGluR4 receptor is mainly located in presynaptic endings of nerve endings. Expression of mGluR4 has been demonstrated in multiple brain regions with high expression within the basal ganglia and cerebellum among other brain regions. Due to expression of mGluR4 within relevant brain circuitries and its role to modulate transmitter release, mGluR4 modulators are considered to have impact on motor control (including Parkinsons Disease), impulse control, learning and memory, anxiety, pain, cerebellar functions, epilepsy, modulation of excitation/inhibition balance, which is of crucial importance for information processing (Marino et al. Ann NY Acad Sci, 2003, 1003: 435-437; Isherwood et al. Neuropharmacology 2017, 123: 249-260; Makoff et al. Mol Brain Res, 1996, 37: 239-248; Davis et al. Neuropharmacology 2013, 66: 365-372; Iscru et al. Genes Brain Behav. 2013, 12: 615-625; Szczurowska and Mareš, Physiol Res, 2012, 61: 619-628) but is not limited to these actions.

As mGluR4 has been reported to be also expressed in peripheral tissue like islets of Langerhans, but not limited to, it is considered that antagonists of mGluR4 function will also have therapeutic effect in disorders including but not limited to metabolic disorders, gastrointestinal disorders, and cancer (Chang et al. Clin Cancer Res. 2005, 11: 3288-3295; Uhera et al. Diabetes 2004, 53: 998-1006; Nunez-Salces et al._Neurogastroenterol Motil 2020, 32).

As mGluR4 has been reported to be also expressed in vagal afferents as well as within central satiety pathways and brain circuits, it is considered that antagonists of mGluR4 function may also have therapeutic effect in disorders including but not limited to overweight and obesity (Blackshow et al. Front Neurosci 2011, 5: 40; 1-7; Page et al. Br J Pharmacol. 2012, 166: 1537-1558).

WO21028512 describes arylsulfonamides as mGluR4 NAMs. However, the activity of those compounds seems too low to be applicable as drug, especially so since acidic arylsulfonamides might additionally be subject to efflux at the blood brain barrier, which limits their brain exposure for CNS applications.

### DETAILED DESCRIPTION OF THE INVENTION

The present invention provides novel substituted pyrazine-carboxamide derivatives of formula I

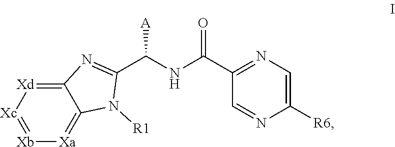

in which
A represents C₁-C₆-alkyl, C₃-C₆-cycloalkyl, C₃-C₅-cycloalkyl-C₁-C₂-alkyl-, C₁-C₃-alkyl-O—C₁-C₃-alkyl-, 4-6-membered heterocycloalkyl-, 4-6-membered heterocycloalkyl-C₁-C₃-alkyl-, which latter groups are optionally substituted with 1-4 substituents chosen from C₁-C₄-alkyl, C₁-C₄-alkoxy, hydroxy, fluoro;
Xa represents either N or C—R²
Xb represents either N or C—R³;
Xc represents either N or C—R⁴;
Xd represents C—R⁵;

Provided that one of Xa, Xb and Xc represents N;
R¹ represents C₁-C₇-alkyl, C₁-C₃-alkyl-O—C₁-C₃-alkyl-, C₃-C₇-cycloalkyl, 4-6-membered heterocycloalkyl, C₃-C₇-cycloalkyl-C₁-C₃-alkyl-, 4-6-membered-heterocycloalkylmethyl-, C₅-C₆-heterocycloalkylethyl-, which latter groups are optionally substituted with 1-4 substituents chosen from C₁-C₄-alkyl, C₁-C₄-alkoxy, C₃-C₇-cycloalkoxy, hydroxy, fluoro;
R², R³, R⁴ and R⁵ independently of each other represent hydrogen, halogen, cyano, C₁-C₄-alkyl, C₁-C₃-alkyl-O—C₁-C₃-alkyl-, C₃-C₆-cycloalkyl, 4-6-membered C₄-C₆-heterocycloalkyl-, C₁-C₄-alkoxy-, C₃-C₅-cycloalkoxy-, which latter six groups are optionally substituted with 1-4 substituents chosen from C₁-C₄-alkyl, C₁-C₄-alkoxy, hydroxy, fluoro;
R⁶ represents halogen, C₁-C₃-alkyl optionally substituted with 2-3 fluorine atoms;
or a physiologically acceptable salt thereof.

In another embodiment, the present invention provides compounds of formula Ia, Ib and Ic

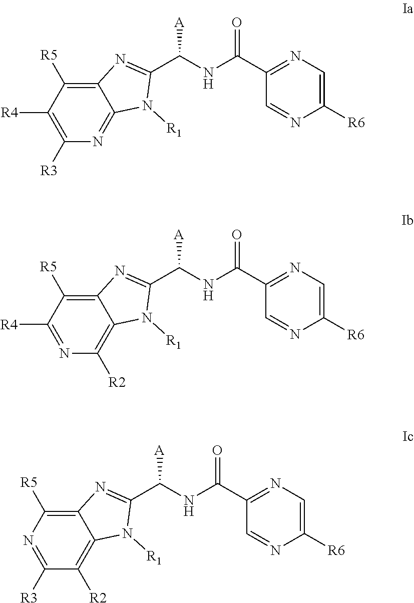

in which
A represents C₁-C₆-alkyl, C₃-C₆-cycloalkyl, C₃-C₅-cycloalkyl-C₁-C₂-alkyl-, C₁-C₃-alkyl-O—C₁-C₃-alkyl-, 4-6-membered heterocycloalkyl-, 4-6-membered heterocycloalkyl-C₁-C₃-alkyl-, which latter groups are optionally substituted with 1-4 substituents chosen from C₁-C₄-alkyl, C₁-C₄-alkoxy, hydroxy, fluoro;
R¹ represents C₁-C₇-alkyl, C₁-C₃-alkyl-O—C₁-C₃-alkyl-, C₃-C₇-cycloalkyl, 4-6-membered heterocycloalkyl, C₃-C₇-cycloalkyl-C₁-C₃-alkyl-, 4-6-membered-heterocycloalkylmethyl-, C₅-C₆-heterocycloalkylethyl-, which latter groups are optionally substituted with 1-4 substituents chosen from C₁-C₄-alkyl, C₁-C₄-alkoxy, C₃-C₇-cycloalkoxy, hydroxy, fluoro;
R², R³, R⁴ and R⁵ independently of each other represent hydrogen, halogen, cyano, C₁-C₄-alkyl, C₁-C₃-alkyl-O—C₁-C₃-alkyl-, C₃-C₆-cycloalkyl, 4-6-membered C₄-C₆-heterocycloalkyl-, C₁-C₄-alkoxy-, C₃-C₅-cycloalkoxy-, which latter six groups are optionally substituted with 1-4 substituents chosen from C₁-C₄-alkyl, C₁-C₄-alkoxy, hydroxy, fluoro;
R⁶ represents halogen, C₁-C₃-alkyl optionally substituted with 2-3 fluorine atoms;
or a physiologically acceptable salt thereof.

In another embodiment, in the general formula I, according to any one of the preceding embodiments
A represents C₁-C₃-alkyl, C₃-C₆-cycloalkyl, C₃-C₅-cycloalkylmethyl-, tetrahydrofuranyl-, tetrahydropyranyl-, 1,4-dioxanyl-, tetrahydrofuranylmethyl-, tetrahydropyranylmethyl-, 1,4-di-oxanylmethyl-, C₁-C₂-alkyl-O—C₁-C₃-alkyl-, which latter groups are optionally substituted with 1-4 substituents chosen from methyl, methoxy, hydroxy, fluoro.

In a further embodiment, in the general formula I, according to any one of the preceding embodiments
R¹ represents C₁-C₃-alkyl, C₁-C₂-alkyl-O—C₁-C₃-alkyl-, C₃-C₄-cycloalkyl, C₄-C₅-heterocycloalkyl, C₃-C₄-cycloalkyl-O—C₁-C₃-alkyl-, which latter groups are optionally substituted with 1-4 substituents chosen from C₁-C₄-alkyl, C₁-C₄-alkoxy, C₃-C₄-cycloalkoxy, hydroxy, fluoro.

In a further embodiment, in the general formula I, according to any one of the preceding embodiments
R², R³, R⁴ and R⁵ independently of each other represent hydrogen, fluoro, chloro, bromo, cyano, methyl, cyclopropyl, methoxy, which latter three groups are optionally substituted with 2-3 fluoro substituents.

In a further embodiment, in the general formula I, according to any one of the preceding embodiments
R⁶ represents C₁-C₃-alkyl optionally substituted with 2-3 fluorine atoms.

In another embodiment, in the general formula I, according to any one of the preceding embodiments
A represents a group chosen from the group comprising

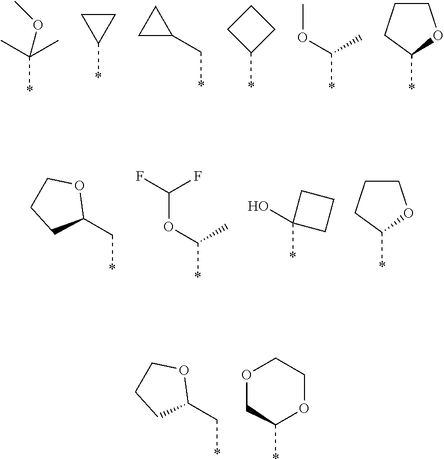

In another embodiment, in the general formula I, according to any one of the preceding embodiments
R¹ represents a substituent chosen from the group consisting of ethyl, —CH₂—CHF₂, iso-propyl.

In another embodiment, in the general formula I, according to any one of the preceding embodiments
R² represents hydrogen.

In another embodiment, in the general formula I, according to any one of the preceding embodiments
R³ represents hydrogen, methyl and trifluromethyl.

In another embodiment, in the general formula I, according to any one of the preceding embodiments
R⁴ represents hydrogen, fluoro, chloro, bromo, cyano, methyl and trifluromethyl.

In another embodiment, in the general formula I, according to any one of the preceding embodiments
R⁵ represents hydrogen, methyl and methoxy.

In another embodiment, in the general formula I, according to any one of the preceding embodiments
R⁶ represents methyl, trifluromethyl and —CF₂H.

Compounds of the present invention are potent mGluR4 negative modulators inhibiting the function of mGluR4 thereby blocking glutamate induced intracellular cAMP lowering.

The present invention thus provides compounds for use in the treatment of a mGluR4 mediated disorder.

The present invention further provides methods of treating a mGluR4 mediated disorder in a human subject comprising administering to the subject a compound or composition of a compound of the present invention or a pharmaceutically acceptable salt thereof.

In one aspect, the invention relates to a method for treating a condition for which reduced mGluR4 activity can reduce the severity of the condition, by administering a compound inhibiting mGluR4 function, such as a compound as described herein that inhibits glutamate induced intracellular cAMP lowering. Described herein are compounds, which are antagonists of mGluR4 function that have a measured IC₅₀ for inhibition of mGluR4 of 50 nanomolar or less.

In another aspect, the compounds described herein, which are antagonists of mGluR4 function can be used to inhibit a function of mGluR4, for example a mGluR4-mediated glutamate induced intracellular cAMP lowering. In some embodiments, the compounds described herein can be used to inhibit a mGluR4 mediated glutamate induced intracellular cAMP lowering in vitro, for example in cells in culture. In other embodiments, the compounds described herein can be used to inhibit a mGluR4 mediated glutamate induced intracellular cAMP lowering in vivo.

### Definitions

Terms not specifically defined herein should be given the meanings that would be given to them by one skilled in the art in light of the disclosure and the context.

The terms “negative modulator”, “antagonist” and “inhibitor” are used interchangeably to refer to an agent that decreases or suppresses a biological activity, such as the reduction of an activity of a receptor, and comprise negative allosteric modulators (NAM). mGluR4 receptors as described herein include homomultimeric and heteromultimeric structures (e.g. homomultimeric mGluR4 and heteromeric mGluR4-mGluR2). Inhibitors of mGluR4 function include inhibitors having any combination of the structural and/or functional properties disclosed herein.

An “effective amount” of an (mGluR4 antagonist), with respect to the subject methods of inhibition or treatment, refers to an amount of the antagonist in a preparation which, when applied as part of a desired dosage regimen brings about a desired clinical or functional result. Without being bound by theory, an effective amount of a mGluR4 antagonist for use in the methods of the present invention includes an amount of a mGluR4 antagonist effective to decrease one or more in vitro or in vivo functions of a mGluR4 receptor. Exemplary functions include, but are not limited to, changed intracellular cAMP, or synaptic transmitter release, or changed neuronal activity or modulation of impulsive behavior. Compounds that antagonize mGluR4 function include compounds that antagonize an in vitro or in vivo functional activity of mGluR4. When a particular functional activity is only readily observable in an in vitro assay, the ability of a compound to inhibit mGluR4 function in that in vitro assay serves as a reasonable proxy for the activity of that compound. In certain embodiments, an effective amount is an amount sufficient to inhibit a mGluR4-mediated cellular function.

The mGluR4 antagonists for use in the methods of the present invention may be characterized according to their activity, or lack of activity, against one or more receptors. When other receptors are referred to, inhibition of a function of such other receptors is defined similarly. For example, inhibition of a receptor or an activity of a receptor means the antagonist inhibits one or more functional activities of the other receptor. Such functions include e.g. signal transduction across a cellular membrane and/or changes in the intracellular concentration of intracellular substances like cAMP mediated by the particular receptor and subsequent functions like e.g. neurotransmitter release.

The terms “compound” and “agent” are used interchangeably to refer to the negative modulators of the invention.

In the groups, radicals, or moieties defined below, the number of carbon atoms is often specified preceding the group, for example, C1-6-alkyl means an alkyl group or radical having 1 to 6 carbon atoms. In general, for groups comprising two or more subgroups, the last named subgroup is the radical attachment point, for example, the substituent “aryl-C1-3-alkyl-” means an aryl group which is bound to a C1-3-alkyl-group, the latter of which is bound to the core or to the group to which the substituent is attached.

In case a compound of the present invention is depicted in form of a chemical name and as a formula in case of any discrepancy the formula shall prevail.

An asterisk may be used in sub-formulas to indicate the bond which is connected to the core molecule as defined.

### Stereochemistry/Solvates/Hydrates

The compounds described herein can be chiral (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis.

Resolution of racemic mixtures of compounds can be carried out by any of numerous methods known in the art. An example method includes fractional recrystallizaion using a “chiral resolving agent” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as p-camphorsulfonic acid. Other resolving agents suitable for fractional crystallization methods include stereoisomerically pure forms of α-methylbenzylamine (e.g., S- and R-forms, or diastereomerically pure forms), 2-phenylglycinol, norephedrine, ephedrine, N-methylephedrine, cyclohexylethylamine, and 1,2-diaminocyclohexane.

Resolution of racemic mixtures can also be carried out by elution on a column packed with an optically active resolving agent (e.g., dinitrobenzoylphenylglycine). Suitable elution solvent composition can be determined by one skilled in the art. Compounds of the invention also include tautomeric forms, such as keto-enol tautomers.

Unless specifically indicated, throughout the specification and the appended claims, a given chemical formula or name shall encompass tautomers and all stereo, optical and geometrical isomers (e.g. enantiomers, diastereoisomers, E/Z isomers) and racemates thereof, as well as mixtures in different proportions of the separate enantiomers, mixtures of diastereoisomers, or mixtures of any of the foregoing forms where such isomers and enantiomers exist.

Compounds of the invention can also include all isotopes of atoms occurring in the intermediates or final compounds. For example, the compound of the invention may be radiolabeled with radioactive isotopes, such as for example tritium (³H) or carbon-14 (¹⁴C). All isotopic variations, whether radioactive or not, are intended to be encompassed within the scope of the present invention.

### Salts

The phrase “pharmaceutically acceptable” is employed herein to refer to those compounds, materials, compositions, and/or dosage forms which are, within the scope of sound medical judgment, suitable for use without excessive toxicity, irritation, allergic response, or other problem or complication, and commensurate with a reasonable benefit/risk ratio.

As used herein, “pharmaceutically acceptable salts” refer to derivatives of the disclosed compounds wherein the parent compound forms a salt with an acid or a base.

Examples for acids forming a pharmaceutically acceptable salt with a parent compound containing a basic moiety include mineral or organic acids such as benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid or tartaric acid. Also included are the salts of amino acids such as arginate, and salts of organic acids like glucuronic or galactunoric acids (see, for example, Berge et al., “Pharmaceutical Salts”, Journal of Pharmaceutical Science, 1977, 66, 1-19).

The neutral form of the compounds of the invention is preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

### Halogen

The term halogen generally denotes fluorine, chlorine, bromine and iodine.

### Alkyl

The term “C₁-n-alkyl”, wherein n is an integer from 2 to n, either alone or in combination with another radical denotes an acyclic, saturated, branched or linear hydrocarbon radical with 1 to n C atoms. For example, the term C₁-C₅-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

### Cycloalkyl

The term “C3-n-cycloalkyl”, wherein n is an integer from 4 to n, either alone or in combination with another radical denotes a cyclic, saturated, unbranched hydrocarbon radical with 3 to n C atoms. For example, the term C3-7-cycloalkyl includes cyclopropyl, cyclobutyl, cyclopentyl, cyclohexyl and cycloheptyl.

### Heterocycloalkyl:

The term “heterocycloalkyl” means a saturated or unsaturated mono- or polycyclic-ring systems including aromatic ring system containing one or more heteroatoms selected from N, O or S(O)r, wherein r=0, 1 or 2, consisting of 3 to 14 ring atoms wherein none of the heteroatoms is part of the aromatic ring.

Many of the terms given above may be used repeatedly in the definition of a formula or group and in each case have one of the meanings given above, independently of one another.

According to the invention the compounds of general formula (I) are obtained by methods known per se, for example by the following methods:
(a) The preparation of a compound of general formula (I)

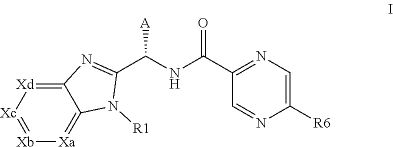

wherein A, Xa, Xb, Xc, Xd and R¹ to R⁶ are defined as described in embodiment 1, and which may optionally be protected at any amino, hydroxy, carboxy or thiol groups durch common protective groups such as for example those described in T. W. Greene, P. G. M. Wuts in “Protective Groups in Organic Synthesis”, Wiley, 1991 and 1999, and the protective groups of which may be cleaved by methods known from the literature,
is described in the examples or may be carried out for example according to the following formula scheme 1.

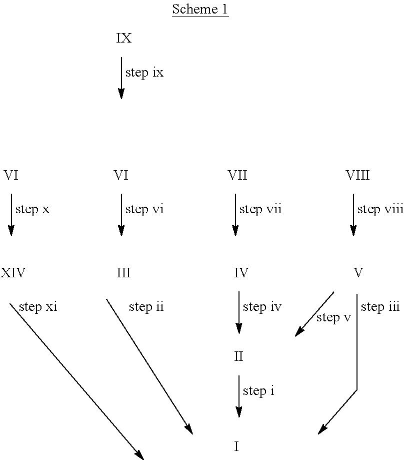

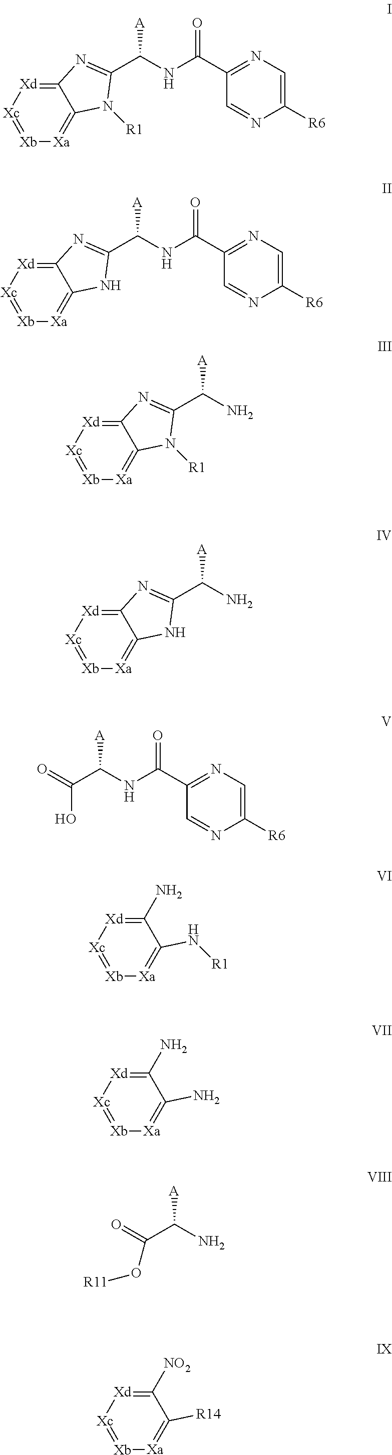

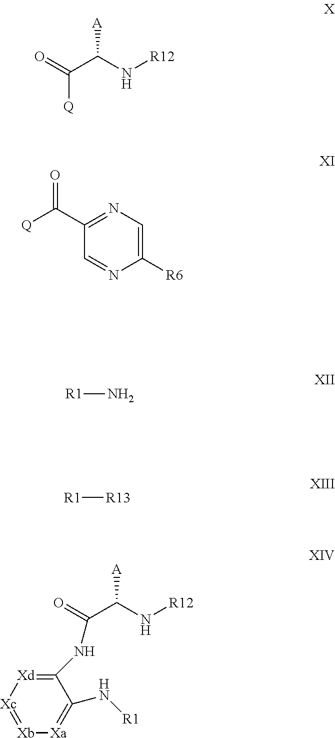

wherein
Q denotes a leaving group or a group which may be converted in-situ into a leaving group, such as for example a halogen atom, a hydroxy, C₁₋₄-alkyloxy, alkyloxycarbonyloxy, 4-pentafluorophenyloxy, nitrophenyloxy, a trichloromethyl or acyloxy group or together with the carbonyl group denotes an alkali carboxylate group, and
R11 denotes a protective group for the carboxylate function known from the literature, such as for example a tert.-butyl, methyl, ethyl, allyl or benzyl group, and
R12 denotes a protective group for the amino function known from the literature, such as for example a tert.-butoxycarbonyl, benzyloxycarbonyl or a trifluoroacetyl group, and
R13 denotes a leaving group for alkylating reactions, such as for example a iodine or bromine atom or tosylate or mesylate group, and
R14 denotes a leaving group for nucleophilic aromatic substitution reactions, such as for example a fluorine or chlorine atom.

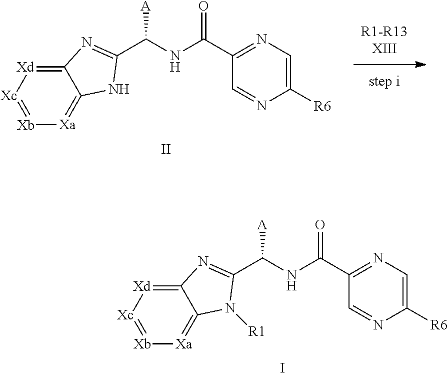

The reaction step i (substitution) shown in Scheme 1 may be carried out in the manner described in the Examples or according to the conditions known from the literature, for example as follows:
Compounds of general formula II are mixed with compounds of general formula XIII in a solvent such as methylene chloride, chloroform, carbon tetrachloride, diethylether, tetrahydrofuran, dioxane, benzene, toluene, acetonitrile, dimethylformamide, dimethylsulphoxide, sodium hydroxide solution or sulpholane, optionally in the presence of an inorganic or organic base like potassium carbonate, sodium hydride, triethylamine or Hunig's base at temperatures between −20 and 200° C., but preferably at temperatures between −10 and 100° C.

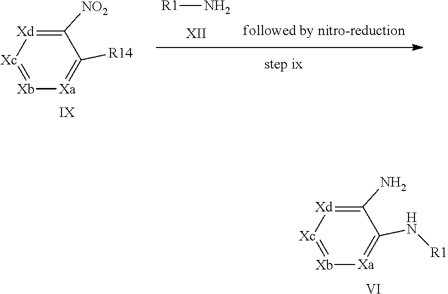

The reaction step ix (substitution followed by nitro-reduction) shown in Scheme 1 may be carried out in the manner described in the Examples or according to the conditions known from the literature, for example as follows:
Substitution of a substrate IX with an amine XII like described above, followed by a nitro-reduction like described below:
A nitro reduction to an amine group can be achieved in an aqueous solvent, e.g. in water, isopropanol/water, tetrahydrofuran/water or dioxane/water, or in a solvent such as diethylether, tetrahydrofuran, dioxane, benzene, toluene, in the presence of an acid such as trifluoroacetic acid, hydrochloric acid or sulphuric acid and in the presence of a reductive metal like zink, iron, magnesium or calcium or in the presence of an reductive agent like triphenyl phosphine or lithium alanate, at temperatures between −40 and 100° C., preferably at temperatures between −10 and 50° C. Alternatively, reduction can be achieved with hydrogen in the presence of a catalyst such as palladium/charcoal, Raney nickel or Platinum in a solvent such as tetrahydrofuran, methanol, ethanol, ethyl acetate, dimethylformamide, dimethylformamide/acetone or glacial acetic acid, optionally with the addition of an acid such as hydrochloric acid at temperatures between −20 and 50° C., but preferably at 0° C. to ambient temperature, and at a hydrogen pressure of 1 to 7 bar, but preferably, 1 to 5 bar.

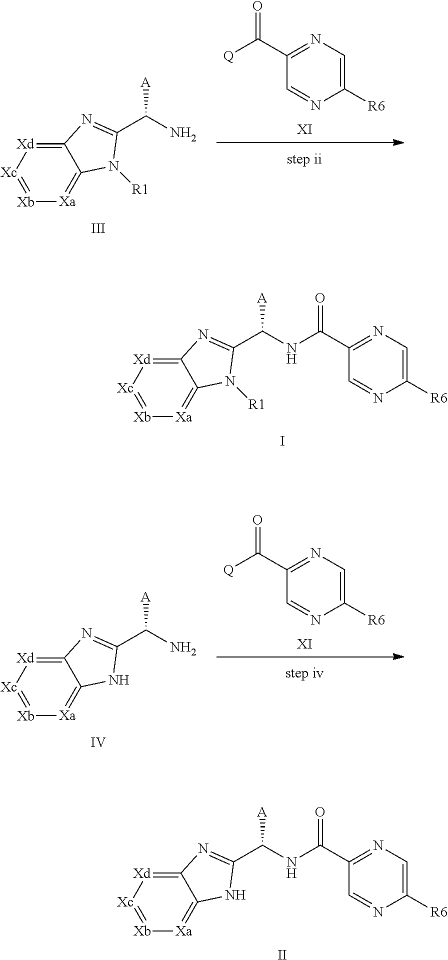

The reaction steps ii and iv (acylation) may be carried out in the manner described in the Examples or according to the conditions known from the literature, for example as follows:
by acylating an amine (III or IV) with an optionally activated carboxylic acid (XI):
The acylation is conveniently carried out with a corresponding halide or anhydride in a solvent such as methylene chloride, chloroform, carbon tetrachloride, ether, tetrahydrofuran, dioxane, benzene, toluene, acetonitrile, dimethylformamide, dimethylsulphoxide, sodium hydroxide solution or sulpholane, optionally in the presence of an inorganic or organic base like potassium carbonate, sodium hydride, triethylamine or Hünig's base at temperatures between −20 and 200° C., but preferably at temperatures between −10 and 100° C.
The acylation may however also be carried out with the free acid optionally in the presence of an acid-activating agent or a dehydrating agent, for example in the presence of ethyl-1-ethoxy-1,2-dihydroquinoline-1-carboxylate, isobutyl chloroformate, thionyl chloride, trimethylchlorosilane, hydrogen chloride, sulphuric acid, methanesulphonic acid, p-toluenesulphonic acid, phosphorus trichloride, phosphorus pentoxide, propanephosphonic acid cycloanhydride, N,N′-dicyclohexylcarbodiimide, N,N′-dicyclohexylcarbodiimide/camphorsulphonic acid, N,N′-dicyclohexylcarbodiimide/N-hydroxysuccinimide or 1-hydroxy-benzotriazole, N,N′-carbonyldiimidazole, 0-(benzotriazol-1-yl)-N,N,N′,N′-tetramethyl-uronium tetrafluoroborate/N-methylmorpholine, 0-(benzotriazol-1-yl)-N,N,N′,N′-tetramethyl-uronium tetrafluoroborate/N-ethyldiisopropylamine, O-(7-azabenzotriazol-1-yl)-N,N,N′,N′-tetramethyluronium-hexafluorophosphate/N-methylmorpholine, 0-pentafluorophenyl-N,N,N′,N′-tetramethyluronium-hexafluorophosphate/triethylamine, N,N′-thionyldiimidazole or triphenylphosphine/carbon tetrachloride, optionally with the addition of an auxiliary base such as sodium hydroxide solution, caesium, potassium or sodium carbonate or hydrogen carbonate or an amine base such as pyridine, triethylamine, N-methylmorpholine or diisopropylethylamine, at temperatures between −20 and 200° C., but preferably at temperatures between −10 and 160° C.
Other methods of amide coupling are described for example in P. D. Bailey, I.D. Collier, K. M. Morgan in “Comprehensive Functional Group Interconversions”, Vol. 5, page 257ff., Pergamon 1995, or in the Houben-Weyl Supplementary Volume 22, published by Thieme, 2003, and the literature cited therein.

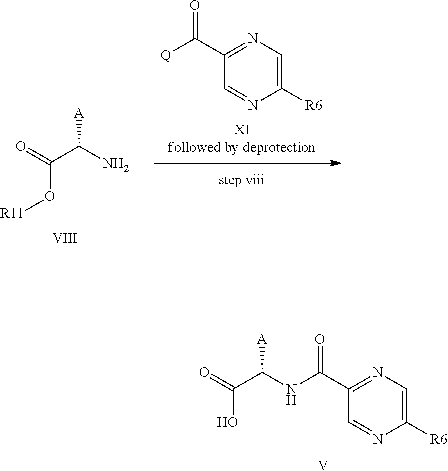

The reaction steps viii (acylation followed by deprotection) may be carried out in the manner described in the Examples or according to the conditions known from the literature, for example as follows:
Acylation of an amine-carrying substrate VIII with a reagent XI like described above, followed by cleaving a protective group like described below:
Any protecting group used may optionally subsequently be cleaved for example by hydrolysis in an aqueous solvent, e.g. in water, isopropanol/water, tetrahydrofuran/water or dioxane/water, in the presence of an acid such as trifluoroacetic acid, hydrochloric acid or sulphuric acid or in the presence of an alkali metal base such as lithium hydroxide, sodium hydroxide or potassium hydroxide or by ether splitting, e.g. in the presence of iodotrimethylsilane, at temperatures between 0 and 100° C., preferably at temperatures between 1° and 50° C.
However, a benzyl, methoxybenzyl or benzyloxycarbonyl group is cleaved hydrogenolytically, for example, e.g. with hydrogen in the presence of a catalyst such as palladium/charcoal in a solvent such as tetrahydrofuran, methanol, ethanol, ethyl acetate, dimethylformamide, dimethylformamide/acetone or glacial acetic acid, optionally with the addition of an acid such as hydrochloric acid at temperatures between 0 and 50° C., but preferably at ambient temperature, and at a hydrogen pressure of 1 to 7 bar, preferably, however, 1 to 5 bar.
However, a protective group may also be cleaved by the methods described by T. W. Greene, P. G. M. Wuts in “Protective Groups in Organic Synthesis”, Wiley, 1991 and 1999.

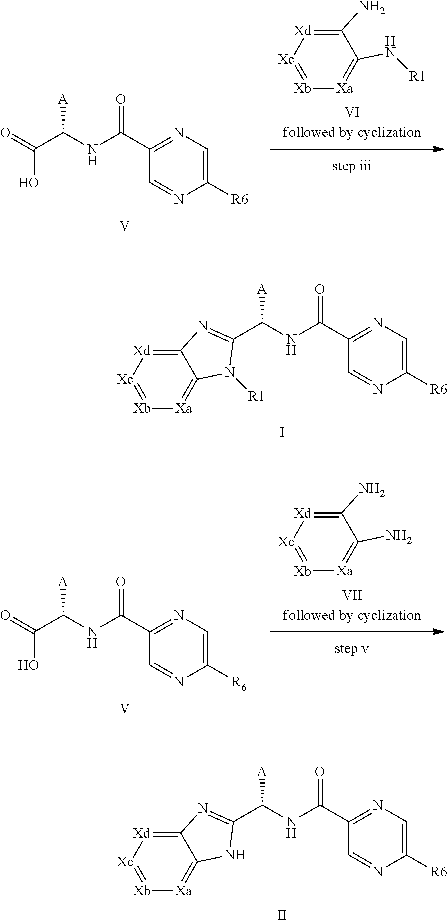

The reaction steps iii and v (acylation followed by cyclization) may be carried out in the manner described in the Examples or according to the conditions known from the literature, for example as follows:
Acylation of an amine-carrying substrate V with a reagent VI or VII like described above, followed by cyclization like described below:
The cyclisation is conveniently carried out in a solvent or mixture of solvents such as ethanol, isopropanol, acetic acid, benzene, chlorobenzene, toluene, xylene, glycol, glycolmonomethylether, diethyleneglycoldimethylether, sulpholane, dimethylformamide or tetraline, dimethylsulphoxide, methylene chloride, chloroform, tetrachloromethane, for example at temperatures between 0 and 250° C., but preferably between 2° and 100° C., optionally in the presence of a condensing agent such as phosphorus oxychloride, thionyl chloride, sulphuryl chloride, sulphuric acid, p-toluenesulphonic acid, methanesulphonic acid, hydrochloric acid, phosphoric acid, polyphosphoric acid, acetic acid, acetic anhydride, N,N′-dicyclohexylcarbodiimide or optionally also in the presence of a base such as potassiumethoxide or potassium-tert.-butoxide or in the presence of a metal salt like lithium bromide, aluminum bromide, zinc bromide or aluminum-donated montmorillonite clay. However, the cyclisation may also be carried out without a solvent and/or condensing agent.

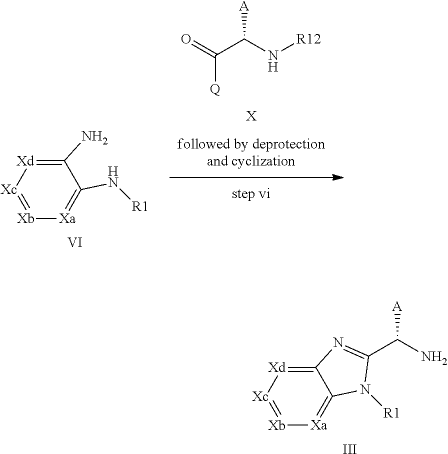

The reaction steps vi (acylation followed by deprotection and cyclization) may be carried out in the manner described in the Examples or according to the conditions known from the literature, for example as follows:
Acylation of an amine-carrying substrate VI with a reagent X like described above, followed by cleaving a protective group like described above, followed by cyclization like described above.

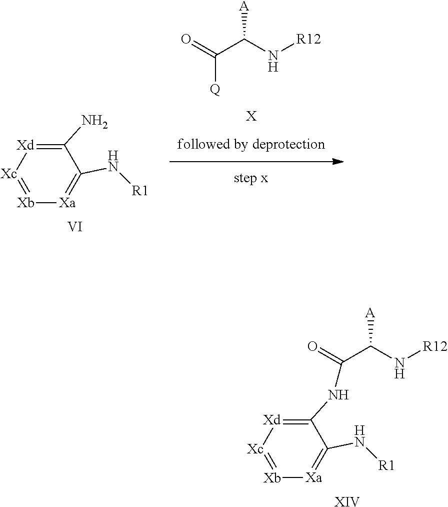

The reaction step x (acylation followed by deprotection) shown in Scheme 1 may be carried out in the manner described in the Examples or according to the conditions known from the literature, for example as follows:
Acylation of a substrate VI with a carboxylic acid or carboxylic acid derivative X like described above, followed by deprotection like described above.

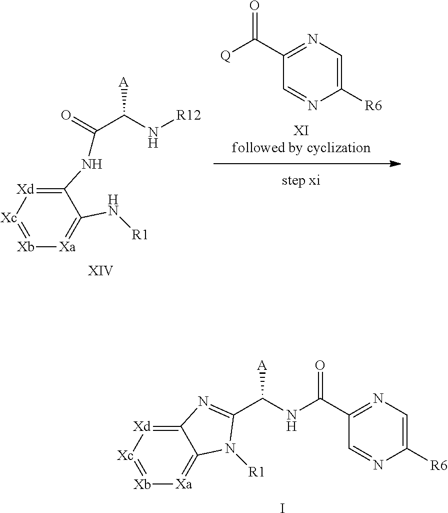

The reaction step xi (acylation followed by cyclization) shown in Scheme 1 may be carried out in the manner described in the Examples or according to the conditions known from the literature, for example as follows:
Acylation of substrate XIV with a carboxylic acid or carboxylic acid derivative XI like described above, followed by cyclization like described above.

The terms “mGluR4”, “mGluR4 protein”, and “mGluR4 receptor” are used interchangeably throughout the application. Unless expressly stated, the term mGluR4 includes homomultimeric structures (e.g. homomultimeric mGluR4) and heteromultimeric structures (e.g. heteromultimeric mGluR4-mGluR2).

### Biological Assays

The biological activity of compounds is determined by the following methods:

A. In Vitro Testing of mGluR4 Potency

The in vitro activity of the compounds according to the invention may be investigated as follows:

The HEK293 cell overexpressing the human metabotropic Glutamate 4 receptor were thawed at 37° C. and immediately diluted with cell culture medium. After centrifugation, the cell pellet is re-suspended in medium and then distributed from a stirred spinner flask into the wells of the assay plate. The plates are incubated for one hour at room temperature before they are incubated for 24 hours at 37° C./5% C₀₂. After washing the cells in the plate three times with 80 uL HBSS/HEPES buffer (10 uL buffer remained in the wells after washing), 5 uL per well of compounds diluted in HBSS/HEPES buffer containing 0.2% BSA (final concentration: 0.1%) and 1 mM IBMX (final concentration: 0.5 mM) are added to the wells of the assay plate. Thereafter 5 uL per well of L-Glutamic acid (final concentration: 10 uM), forskolin (final concentration: 1 uM) and 1 mM IBMX (final concentration: 0.5 mM) dissolved in HBSS/HEPES buffer containing 0.2% BSA (final concentration: 0.1%) are added to the assay plate (final DMSO concentration: 1%). Several wells of the assay plate are used either for the positive and the negative controls or for the cAMP standard curve. The assay plate is incubated for 30 minutes at room temperature. Then 5 ul per well of Anti-cAMP-Antibody-d2 solution and 5 ul per well of cAMP-Europium Cryptate dilution are added to all wells of the plate and the plate is incubated another 60 minutes light protected at room temperature. The emission at 615 nm and 665 nm (Excitation wavelength: 320 nm) are measured on the EnVision™ reader (PerkinElmer). The ratio between the emission at 665 nm and 615 is calculated by the reader. The whole assay is performed in the dark or under green light.

The cAMP standard is prepared by diluting the cAMP stock solution with HBSS/Hepes buffer: 5 μl/well of the cAMP dilutions (in HBSS/Hepes buffer containing 1 mM IBMX and 0.2% BSA-final concentration: 0.5 mM IBMX and 0.1% BSA) are added to 10 ul/well HBSS/Hepes buffer plus 5 ul/well 4% DMSO in HBSS/Hepes containing 0.2% BSA (final DMSO concentration: 1%-like in the wells containing compounds) in the wells of the assay plate. The final cAMP concentrations in the assay plate were: 0, 0.17, 0.69, 2.78, 11.1, 44.5, 178, and 712 nM (two wells/cAMP concentration).

Each assay microtiter plate contained also wells with vehicle controls instead of compound as controls for L-Glutamic acid induced signal (negative control; 100% CTL; 10 uM L-Glutamic acid+1 uM forskolin+0.5 mM IBMX+1% DMSO) and wells with vehicle controls without L-Glutamic acid as controls for non-specific changes in signal (positive control; 0% CTL; 0 uM L-Glutamic acid+1 uM forskolin+0.5 mM IBMX+1% DMSO).

The analysis of the data is performed by the calculation the ratio between the emission at 665 nm and the emission at 615 nm (Em665/Em615 ratio). Thereafter the signals of the compounds are normalized using the positive and negative controls by the following formula:

PoC=100×((Signal Sample−Positive Control)/(Negative Control−Positive Control))

### B. Assessment of Metabolic Stability in Human Liver Microsomes (Human MST)

The metabolic stability of the compounds according to the invention may be investigated as follows:

The metabolic degradation of the test compound is assayed at 37° C. with pooled human liver microsomes. The final incubation volume of 100 μL per time point contains TRIS buffer pH 7.6 at room temperature (0.1 M), MgCI₂ (5 mM), microsomal protein (1 mg/mL) and the test compound at a final concentration of 1 μM. Following a short pre-incubation period at 37° C., the reactions are initiated by addition of beta-nicotinamide adenine dinucleotide phosphate, reduced form (NADPH, 1 mM), and terminated by transferring an aliquot into solvent after different time points. After centrifugation (10000 g, 5 min), an aliquot of the supernatant is assayed by LC-MS/MS for the amount of parent compound. The half-life (t₁/2) is determined by the slope of the semi-logarithmic plot of the concentration-time profile.

C. Assessment of efflux in Madin-Darby canine kidney (MDCK) cells transfected with the Human MDR1 Gene

Apparent permeability coefficients (PE) of the compounds across the MDCK-MDR1 cell monolayers are measured (pH 7.4, 37° C.) in apical-to-basal (AB) and basal-to-apical (BA) transport direction. AB permeability (PEAB) represents drug absorption from the blood into the brain and BA permeability (PEBA) drug efflux from the brain back into the blood via both passive permeability as well as active transport mechanisms mediated by efflux and uptake transporters that are expressed on the MDCK-MDR1 cells, predominantly by the overexpressed human MDR1 P-gp. The compounds are assigned to permeability/absorption classes by comparison of the AB permeabilities with the AB permeabilities of reference compounds with known in vitro permeability and oral absorption in the human. Identical or similar permeabilities in both transport directions indicate passive permeation, vectorial permeability points to additional active transport mechanisms. Higher PEBA than PEAB indicates the involvement of active efflux mediated by MDR1 P-gp. Active transport is concentration-dependently saturable. MDCK-MDR1 cells (1-2×10e5 cells/1 cm2 area) are seeded on filter inserts (Costar transwell polycarbonate or PET filters, 0.4 μm pore size) and cultured (DMEM) for 7 days. Subsequently, the MDR1 expression is boosted by culturing the cells with 5 mM sodium butyrate in full medium for 2 days. Compounds are dissolved in appropriate solvent (like DMSO, 1 −20 mM stock solutions). Stock solutions are diluted with HTP-4 buffer (128.13 mM NaCl, 5.36 mM KCl, 1 mM MgSO₄, 1.8 mM CaCl₂, 4.17 mM NaHCO₃, 1.19 mM Na₂HPO₄×7H₂O, 0.41 mM NaH₂PO₄×H2O, 15 mM HEPES, 20 mM glucose, 0.25% BSA, pH 7.4) to prepare the transport solutions (0.1-300 μM compound, final DMSO<=0.5%). The transport solution (TL) is applied to the apical or basolateral donor side for measuring A-B or B-A permeability (3 filter replicates), respectively. The receiver side contains the same buffer as the donor side. Samples are collected at the start and end of experiment from the donor and at various time intervals for up to 2 hours also from the receiver side for concentration measurement by HPLC-MS/MS or scintillation counting. Sampled receiver volumes are replaced with fresh receiver solution.

### D. Assessment of Efficacy on Impulsive Behavior Tested in the Rat Five Choice Serial Reaction Time Task 5-CSRTT

Assessment of efficacy on motor impulsive behavior may be investigated as follows:

5-CSRTT task training took place according to standard protocols (Isherwood et al.

Neuropharmacology 2017, 123: 249-260). Briefly, rats are trained to nose poke at the location of a light cue presented at 1 of 5 locations on a curved wall of an operant box (Med Associates Inc, St. Albans, Vermont). If a nose poke occurred at the illuminated location during or up to 1 s after stimulus presentation a sugar pellet is delivered in a reward receptacle located across the chamber. Infrared beams in each choice aperture and the reward receptacle allowed for precise detection of the rat at this task associated operanda. Motor impulsive behavior is defined as a response at any nose poke aperture which occurred before onset of the light cue (premature response).

After reaching stable performance, a new analytical approach is applied which revealed trait-like (long-term) stability in the number of premature responses individual animals made across several months. In general, this analysis made it possible to robustly stratify animals into high- and low-impulsive groups based on longitudinal assessment of the number of premature responses they made during training.

Experiments are performed in cross-over such that all experiment subjects received both vehicle and compound, on separate days, with each administration separated by ~2 weeks. The order of vehicle and compound administration is randomized within experimental subjects, while a third group is administered Atomoxetine on both experimental days as a technical control.

As a standardized numerical threshold for impulsivity levels, animals with >40 and <40 premature responses (out of 200 initiated trials) in vehicle are labeled as high and low impulsive, respectively. Importantly, this numerical threshold-based labeling overlapped>80% with the longitudinal analysis of the training data (described above). The high convergence of these two approaches towards stratification allowed us to robustly compare compound effects in stably high- vs stably low-impulsive rats in the 5-CSRTT.

### Biological Data

TABLE 1
In vitro potency of the structurally closest compound disclosed in
WO2019/138017 (as determined in Assay A)

| | | Assay A |
|---|---|---|
| | | mGluR4 |
| Example | Structure | IC50 |
| Inter- mediate 213 in WO2019/ 138017 (regio- isomere 2) | 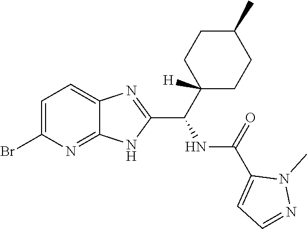 [SMILES: Cn1nccc1C(=O)N[C@H](c1nc2ccc(Br)nc2[nH]1)[C@H]1CC[C@H](C)CC1] | 13.2 μM |

The compounds of the present invention differ structurally from the structurally closest compound in the prior art (I.e. Intermediate 213 in WO 2019/138017) in that the heteromonocycle bound as an carboxamide is a substituted pyrazine (6-membered heteroaryl) rather than a pyrazole moiety (5-membered heteroaryl). Whereas compounds disclosed in WO2019/138017 are immunomodulators (IL-17 modulators), compounds of the present invention unexpectedly are highly potent mGluR4 negative modulators (see Table 2). The structurally closest compound disclosed in WO2019/138017 was tested in Assay A and found to have no therapeutically relevant activity as mGluR4 modulators (Table 1). Unexpectedly, compounds of the present invention are >100 times more potent in Assay A. (Compare data in Tables 1 and 2).

TABLE 2
In vitro potencies of compounds of the present invention as determined in Assay A

| | | Assay A mGluR4 IC₅₀ |
|---|---|---|
| Example | Structure | [μM] |
| 1 | 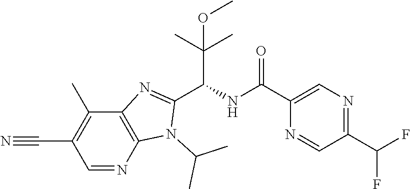 [SMILES: COC(C)(C)[C@H](NC(=O)c1cnc(C(F)F)cn1)c1nc2c(C)c(C#N)cnc2n1C(C)C] | 0.001 |
| 2 | 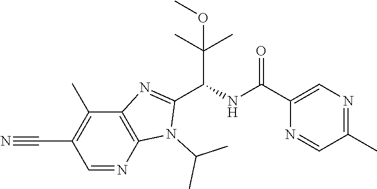 [SMILES: COC(C)(C)[C@H](NC(=O)c1cnc(C)cn1)c1nc2c(C)c(C#N)cnc2n1C(C)C] | 0.002 |
| 3 | 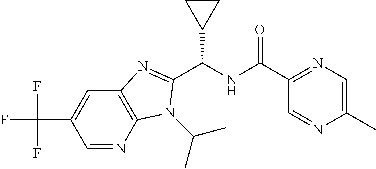 [SMILES: Cc1cnc(C(=O)N[C@H](c2nc3cc(C(F)(F)F)cnc3n2C(C)C)C2CC2)cn1] | 0.002 |
| 4 | 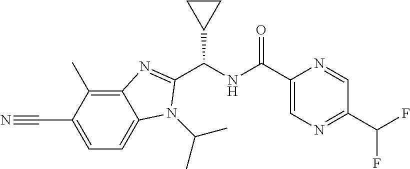 [SMILES: Cc1c(C#N)ccc2c1nc([C@@H](NC(=O)c1cnc(C(F)F)cn1)C1CC1)n2C(C)C] | 0.002 |
| 5 | 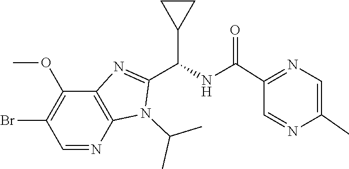 [SMILES: COc1c(Br)cnc2c1nc([C@@H](NC(=O)c1cnc(C)cn1)C1CC1)n2C(C)C] | 0.002 |
| 6 | 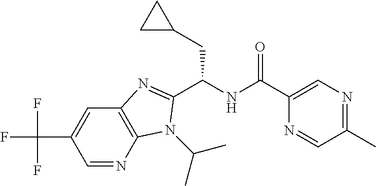 [SMILES: Cc1cnc(C(=O)N[C@@H](CC2CC2)c2nc3cc(C(F)(F)F)cnc3n2C(C)C)cn1] | 0.002 |
| 7 | 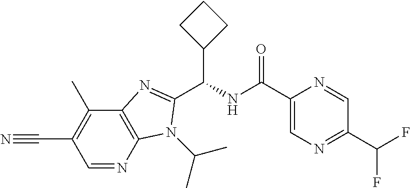 [SMILES: Cc1c(C#N)cnc2c1nc([C@@H](NC(=O)c1cnc(C(F)F)cn1)C1CCC1)n2C(C)C] | 0.003 |
| 8 | 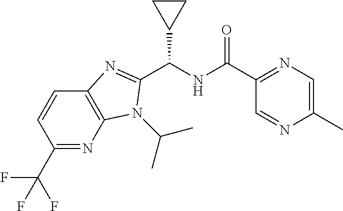 [SMILES: Cc1cnc(C(=O)N[C@H](c2nc3ccc(C(F)(F)F)nc3n2C(C)C)C2CC2)cn1] | 0.003 |
| 9 | 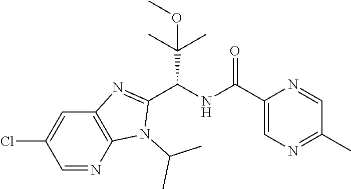 [SMILES: COC(C)(C)[C@H](NC(=O)c1cnc(C)cn1)c1nc2cc(Cl)cnc2n1C(C)C] | 0.003 |
| 10 | 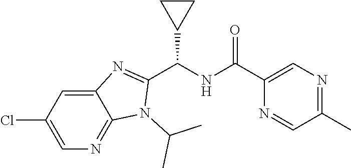 [SMILES: Cc1cnc(C(=O)N[C@H](c2nc3cc(Cl)cnc3n2C(C)C)C2CC2)cn1] | 0.004 |
| 11 | 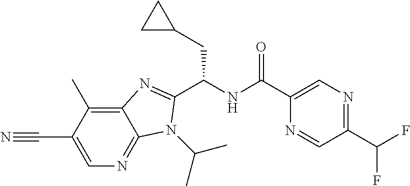 [SMILES: Cc1c(C#N)cnc2c1nc([C@H](CC1CC1)NC(=O)c1cnc(C(F)F)cn1)n2C(C)C] | 0.004 |
| 12 | 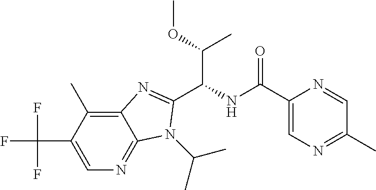 [SMILES: CO[C@H](C)[C@H](NC(=O)c1cnc(C)cn1)c1nc2c(C)c(C(F)(F)F)cnc2n1C(C)C] | 0.004 |
| 13 | 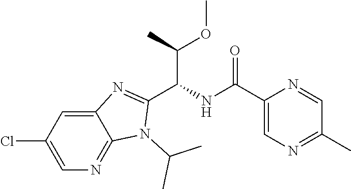 [SMILES: CO[C@H](C)[C@H](NC(=O)c1cnc(C)cn1)c1nc2cc(Cl)cnc2n1C(C)C] | 0.004 |
| 14 | 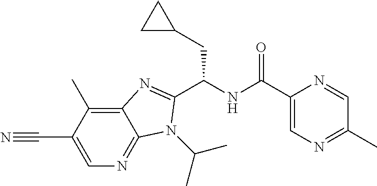 [SMILES: Cc1cnc(C(=O)N[C@@H](CC2CC2)c2nc3c(C)c(C#N)cnc3n2C(C)C)cn1] | 0.005 |
| 15 | 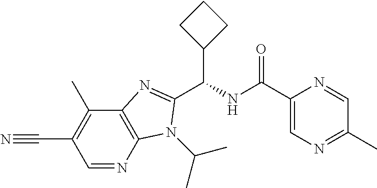 [SMILES: Cc1cnc(C(=O)N[C@H](c2nc3c(C)c(C#N)cnc3n2C(C)C)C2CCC2)cn1] | 0.006 |
| 16 | 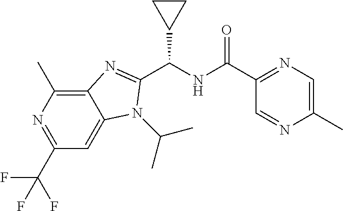 [SMILES: Cc1cnc(C(=O)N[C@H](c2nc3c(C)nc(C(F)(F)F)cc3n2C(C)C)C2CC2)cn1] | 0.013 |
| 17 | 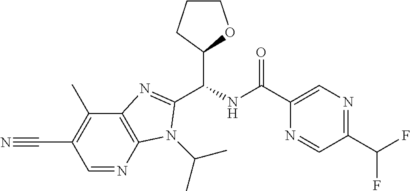 [SMILES: Cc1c(C#N)cnc2c1nc([C@@H](NC(=O)c1cnc(C(F)F)cn1)[C@H]1CCCO1)n2C(C)C] | 0.007 |
| 18 | 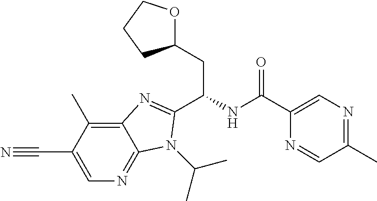 [SMILES: Cc1cnc(C(=O)N[C@@H](C[C@H]2CCCO2)c2nc3c(C)c(C#N)cnc3n2C(C)C)cn1] | 0.007 |
| 19 | 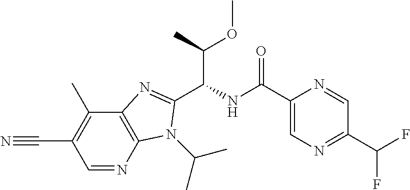 [SMILES: CO[C@H](C)[C@H](NC(=O)c1cnc(C(F)F)cn1)c1nc2c(C)c(C#N)cnc2n1C(C)C] | 0.008 |
| 20 | 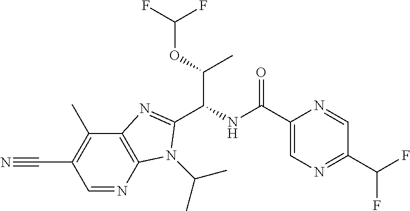 [SMILES: Cc1c(C#N)cnc2c1nc([C@@H](NC(=O)c1cnc(C(F)F)cn1)[C@@H](C)OC(F)F)n2C(C)C] | 0.008 |
| 21 | 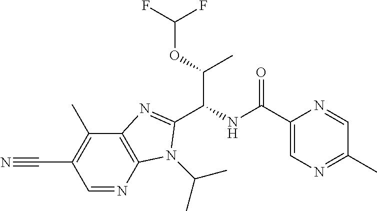 [SMILES: Cc1cnc(C(=O)N[C@H](c2nc3c(C)c(C#N)cnc3n2C(C)C)[C@@H](C)OC(F)F)cn1] | 0.009 |
| 22 | 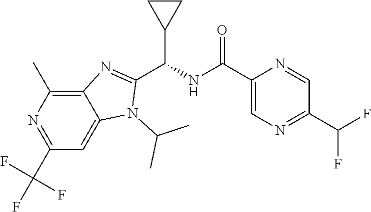 [SMILES: Cc1nc(C(F)(F)F)cc2c1nc([C@@H](NC(=O)c1cnc(C(F)F)cn1)C1CC1)n2C(C)C] | 0.010 |
| 23 | 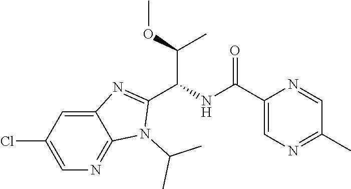 [SMILES: CO[C@@H](C)[C@H](NC(=O)c1cnc(C)cn1)c1nc2cc(Cl)cnc2n1C(C)C] | 0.010 |
| 24 | 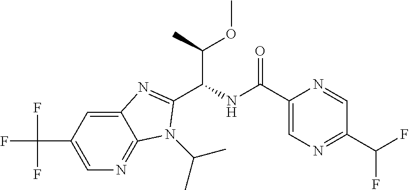 [SMILES: CO[C@H](C)[C@H](NC(=O)c1cnc(C(F)F)cn1)c1nc2cc(C(F)(F)F)cnc2n1C(C)C] | 0.010 |
| 25 | 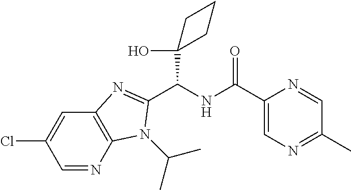 [SMILES: Cc1cnc(C(=O)N[C@H](c2nc3cc(Cl)cnc3n2C(C)C)C2(O)CCC2)cn1] | 0.012 |
| 26 | 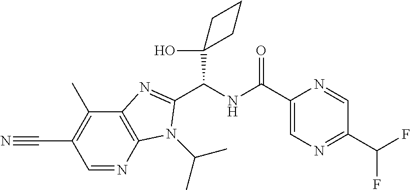 [SMILES: Cc1c(C#N)cnc2c1nc([C@@H](NC(=O)c1cnc(C(F)F)cn1)C1(O)CCC1)n2C(C)C] | 0.013 |
| 27 | 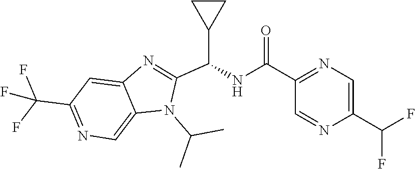 [SMILES: CC(C)n1c([C@@H](NC(=O)c2cnc(C(F)F)cn2)C2CC2)nc2cc(C(F)(F)F)ncc21] | 0.031 |
| 28 | 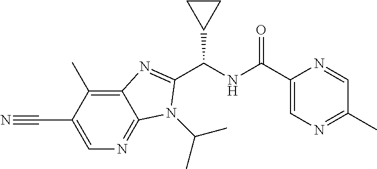 [SMILES: Cc1cnc(C(=O)N[C@H](c2nc3c(C)c(C#N)cnc3n2C(C)C)C2CC2)cn1] | 0.006 |
| 29 | 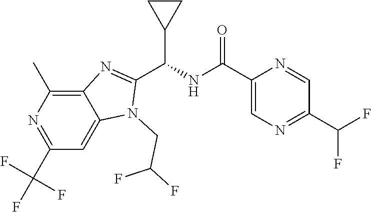 [SMILES: Cc1nc(C(F)(F)F)cc2c1nc([C@@H](NC(=O)c1cnc(C(F)F)cn1)C1CC1)n2CC(F)F] | 0014 |
| 30 | 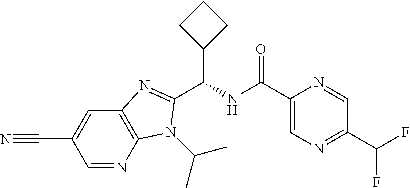 [SMILES: CC(C)n1c([C@@H](NC(=O)c2cnc(C(F)F)cn2)C2CCC2)nc2cc(C#N)cnc21] | 0.014 |
| 31 | 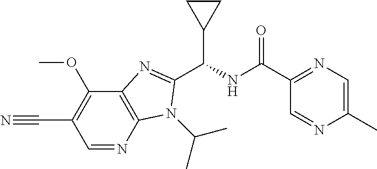 [SMILES: COc1c(C#N)cnc2c1nc([C@@H](NC(=O)c1cnc(C)cn1)C1CC1)n2C(C)C] | 0.014 |
| 32 | 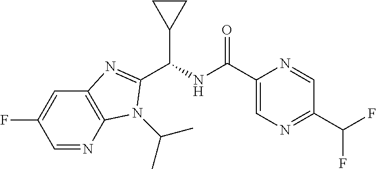 [SMILES: CC(C)n1c([C@@H](NC(=O)c2cnc(C(F)F)cn2)C2CC2)nc2cc(F)cnc21] | 0.015 |
| 33 | 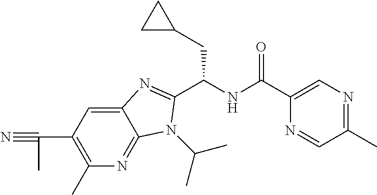 | 0.015 |
| 34 | 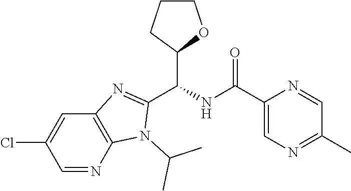 [SMILES: Cc1cnc(C(=O)N[C@H](c2nc3cc(Cl)cnc3n2C(C)C)[C@H]2CCCO2)cn1] | 0.016 |
| 35 | 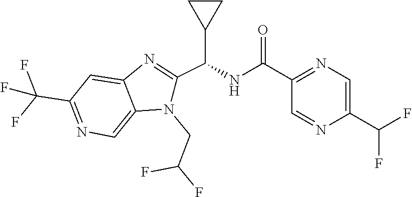 [SMILES: O=C(N[C@H](c1nc2cc(C(F)(F)F)ncc2n1CC(F)F)C1CC1)c1cnc(C(F)F)cn1] | 0.018 |
| 36 | 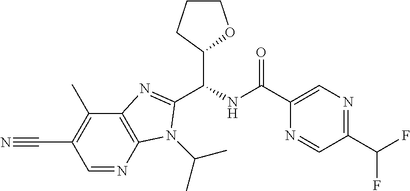 [SMILES: Cc1c(C#N)cnc2c1nc([C@@H](NC(=O)c1cnc(C(F)F)cn1)[C@@H]1CCCO1)n2C(C)C] | 0.018 |
| 37 | 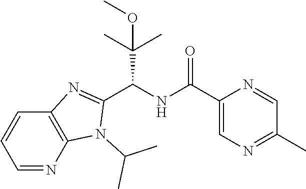 [SMILES: COC(C)(C)[C@H](NC(=O)c1cnc(C)cn1)c1nc2cccnc2n1C(C)C] | 0.019 |
| 38 | 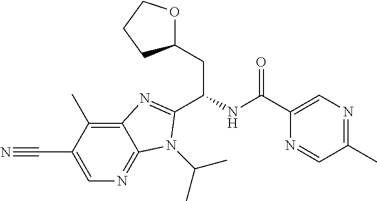 [SMILES: Cc1cnc(C(=O)N[C@@H](C[C@H]2CCCO2)c2nc3c(C)c(C#N)cnc3n2C(C)C)cn1] | 0.019 |
| 39 | 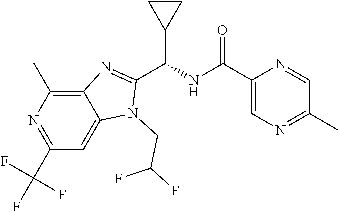 [SMILES: Cc1cnc(C(=O)N[C@H](c2nc3c(C)nc(C(F)(F)F)cc3n2CC(F)F)C2CC2)cn1] | 0.021 |
| 40 | 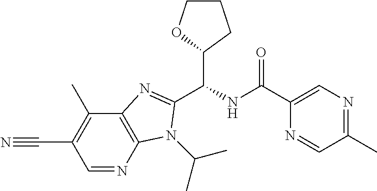 [SMILES: Cc1cnc(C(=O)N[C@H](c2nc3c(C)c(C#N)cnc3n2C(C)C)[C@H]2CCCO2)cn1] | 0.021 |
| 41 | 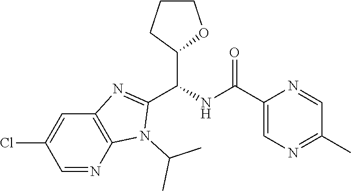 [SMILES: Cc1cnc(C(=O)N[C@H](c2nc3cc(Cl)cnc3n2C(C)C)[C@@H]2CCCO2)cn1] | 0.021 |
| 42 | 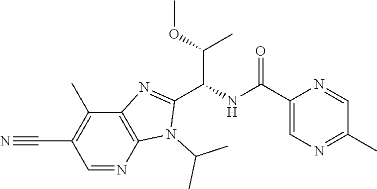 [SMILES: CO[C@H](C)[C@H](NC(=O)c1cnc(C)cn1)c1nc2c(C)c(C#N)cnc2n1C(C)C] | 0.021 |
| 43 | 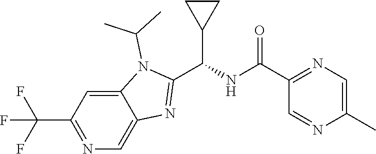 [SMILES: Cc1cnc(C(=O)N[C@H](c2nc3cnc(C(F)(F)F)cc3n2C(C)C)C2CC2)cn1] | 0.024 |
| 44 | 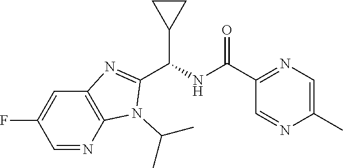 [SMILES: Cc1cnc(C(=O)N[C@H](c2nc3cc(F)cnc3n2C(C)C)C2CC2)cn1] | 0.026 |
| 45 | 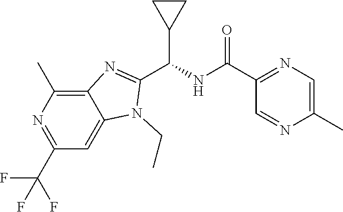 [SMILES: CCn1c([C@@H](NC(=O)c2cnc(C)cn2)C2CC2)nc2c(C)nc(C(F)(F)F)cc21] | 0.033 |
| 46 | 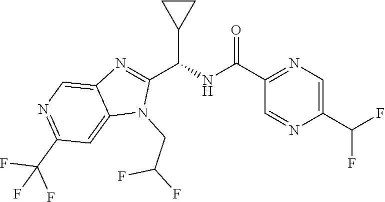 [SMILES: O=C(N[C@H](c1nc2cnc(C(F)(F)F)cc2n1CC(F)F)C1CC1)c1cnc(C(F)F)cn1] | 0.041 |
| 47 | 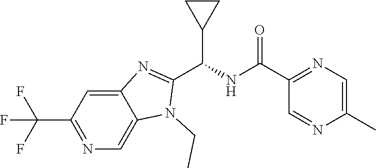 [SMILES: CCn1c([C@@H](NC(=O)c2cnc(C)cn2)C2CC2)nc2cc(C(F)(F)F)ncc21] | 0.041 |
| 48 | 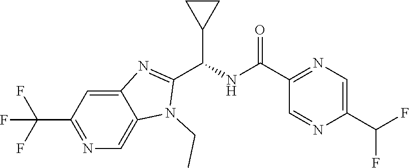 [SMILES: CCn1c([C@@H](NC(=O)c2cnc(C(F)F)cn2)C2CC2)nc2cc(C(F)(F)F)ncc21] | 0.048 |
| 49 | 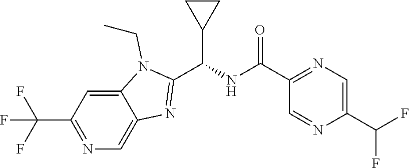 [SMILES: CCn1c([C@@H](NC(=O)c2cnc(C(F)F)cn2)C2CC2)nc2cnc(C(F)(F)F)cc21] | 0.053 |
| 50 | 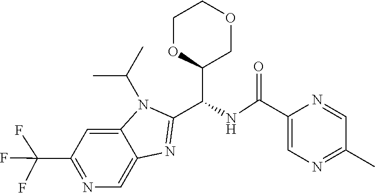 [SMILES: Cc1cnc(C(=O)N[C@H](c2nc3cnc(C(F)(F)F)cc3n2C(C)C)[C@@H]2COCCO2)cn1] | 0.080 |

### Use in Treatment/Method of Use

The present invention is directed to compounds which are useful in the treatment and/or prevention of a disease, disorder and condition wherein the inhibition of mGluR4 activity is of therapeutic benefit, including but not limited to the treatment of psychiatric and neurological conditions associated with impulse control deficits or maladaptive impulsivity. Such impulse control deficits are seen in addictions including substance use disorders; personality disorders such as borderline personality disorder, antisocial personality disorder, conduct disorder, eating disorders such as binge eating disorder, attention deficit hyperactivity disorder, bipolar disorder, stress related disorders such as postraumatic stress disorder, tic disorders like Tourerett's syndrome, other movement disorders such as restless legs syndrome. According to a further aspect of the invention, compounds of the present invention are useful in the treatment of mGluR4 related pathophysiological disturbances, cognition, motivated behaviours/reward, mood and stress, aggression. In addition, there is therapeutic benefit in cancer and related disorders associated with maladaptive tumorgenesis like osteosarcoma.

In view of their pharmacological effect, compounds of the present invention are suitable for use in the treatment and/or of a disease or condition selected from the list consisting of
(1) Disorders associated with malfunction in impulse control such as pathological gambling, trichotillomania, intermittent explosive disorder, conduct disorder, antisocial personality disorder, kleptomania, pyromania, compulsive shopping, internet addiction, sexual compulsion, sexual disorder, sexual dysfunction, psychosexual disorder, eating disorders, such as binge eating, bulimia nervosa, anorexia nervosa, other specified feeding or eating disorders, obesity, overweight, cachexia, appetite/taste disorders, vomiting, nausea, Prader-Willi-syndrome, hyperphagia, appetite/taste disorders, bipolar disorder, posttraumatic stress disorder;
(2) Substance abuse/dependence/seeking or addiction as well as relapse prevention (including but not limited to drugs, such as cocaine, opiates such as morphine, barbiturates, benzodiazepines, amphetamines, nicotine/tobacco and other psychostimulants), alcoholism and alcohol-related disorders, drug abuse or addiction or relapse, tolerance to narcotics or withdrawal from narcotics;
(3) Psychiatric and neurological conditions like attention deficit hyperactivity disorder, conduct disorders, attention problems and related disorders, sleep disorders, anxiety disorders such as generalized anxiety disorder, panic disorder, phobias, post-traumatic stress disorder, schizophrenia, Alzheimer's disease, Parkinson's disease, Huntington's disease and Gilles de la, restless legs syndrome, dementia, dyskinesia, severe mental retardation, neurodegenerative disorders including nosological entities such as disinhibition-dementia-parkinsonism-amyotrophy complex, pallido-ponto-nigral degeneration, Mood disorders, bipolar disorder, mania, depression, manic depression, borderline personality disorder, antisocial personality disorder, aggression such as impulsive aggression, suicidality, frontotemporal dementia, obsessive compulsive disorder, delirium, affective neurosis/disorder, depressive neurosis/disorder, anxiety neurosis, dysthymic disorder, neurological diseases, such as cerebral oedema and angioedema, cerebral dementia like e.g. Parkinson's and Alzheimer's disease, senile dementia; multiple sclerosis, epilepsy, temporal lobe epilepsy, drug resistant epilepsy, seizure disorders, stroke, myasthenia gravis, brain and meningeal infections like encephalomyelitis, meningitis, HIV as well as schizophrenia, delusional disorders, autism, affective disorders and tic disorders including but not limited to Tourette Syndrome and other movement disorders, dpilepsia, chronic pain;
(4) Cognitive dysfunction in psychiatric or neurological disorder, cognitive impairments associated with schizophrenia, Alzheimer's disease and other neurological and psychiatric disorders;
(5) Personality disorders such as borderline personality disorder, antisocial personality disorder, paranoid personality disorder, schizoid and schizotypal personality disorder, histrionic personality disorder, narcissistic personality disorder, avoidant personality disorder, dependent personality disorder, other specified and non-specified personality disorders;
(6) sleep disorders such as narcolepsy, jetlag, sleep apnea, insomnia, parasomnia, disturbed biological and circadian rhythms, sleep disturbances associated with psychiatric and neurological disorders;
(7) Non-neuronal conditions including metabolic conditions like diabetes, insulin resistance, metabolic syndrome, overweight, obesity, as well as use for weight reduction, cosmetic weight loss, relapse prevention during or after obesity treatment, body weight maintenance, emesis, disorders associated with malfunction of the cardiovascular-vascular system and disorders associated with maladaptive blood pressure control like hypertension or hypotension;
(8) Cancer and related disorders associated with maladaptive tumorgenesis like osteosarcoma, breast cancer, ependymoma, bladder cancer, colorectal cancer.

The applicable daily dose of compounds of the present invention may vary from 0.1 to 2000 mg. The actual pharmaceutically effective amount or therapeutic dose will depend on factors known by those skilled in the art such as age and weight of the patient, route of administration and severity of disease. In any case, the drug substance is to be administered at a dose and in a manner which allows a pharmaceutically effective amount to be delivered that is appropriate to the patient's condition.

### Pharmaceutical Compositions

Suitable compositions for administering the compounds of the present invention will be apparent to those with ordinary skill in the art and include for example tablets, pills, capsules, suppositories, lozenges, troches, solutions, syrups, elixirs, sachets, injectables, inhalatives, and powders. The content of the pharmaceutically active compound(s) may vary in the range from 0.1 to 95 wt.-%, preferably 5.0 to 90 wt.-% of the composition as a whole.

Suitable tablets may be obtained, for example, by mixing a compound of the present invention with known excipients, for example inert diluents, carriers, disintegrants, adjuvants, surfactants, binders and/or lubricants and compressing the resulting mixture to tablets.

### Combination Therapy

Compounds according to the present invention can be combined with other treatment options known to be used in the art in connection with a treatment of any of the indications the treatment of which is in the focus of the present invention.

Among such active pharmaceutical ingredients or treatment options that are considered suitable for combination with the compounds and the treatment according to the present invention are antidepressants, mood stabilizers, typical and atypical antipsychotics, anxiolytics, antiepileptic drugs, anti-Parkinsons medication, sleeping agents, cognitive enhancers, stimulants, medication for attention deficit hyperactivity disorder, additional psychoactive drugs, anti-inflammatory drugs, analgesic drugs, chemotherapeutic drugs, as well as combination with treatment options used for metabolic disorders, liver diseases and kidney diseases.

### EXPERIMENTAL SECTION

### List of Abbreviations

% Sol percentage of solvent
μL microliter
ACN acetonitrile
AcOH acetic acid
aq. Aqueous
Boc tert.-butyloxycarbonyl
Boc₂O Di-tert.-butyl-dicarbonate
chir. chiral
CIP 2-chloro-1,3-dimethyl-2-imidazolinium hexafluorophosphate
conc. concentrated
d day
DA Diode Array
DAD Diode array detector
DCM dichloromethane
DMF N,N-dimethylformamide
ELSD Evaporative Light Scattering Detector
EtOAc ethyl acetate
ETOH ethanol
g gram
h hour
half-conc. half concentrated
HPLC high performance liquid chromatography
i. vac. in vacuo
IPA Isopropylic Alcohol
M molar
MeOH methanol
MEOH methanol
mg milligram
min minute
ml milliliter
mL milliliter
MS Mass Spectrometer
N normal
NBS N-Bromo-succinimide
NMM N-methyl-morpholine
NMP N-Methylpyrrolidone
PE Petrolether
PPA 1-propanephosphonic acid cyclic anhydride
prep. Preparative
PSI pound per square inch
quant. quantitative
Rf retarding front
RT retention time
sat. saturated
scCO2 supercritical carbon dioxide
SFC supercritical fluid chromatography
TBTU o-(Benzotriazol-1-yl)-N,N,N,N-tetramethyluronium-tetrafluoroborat
TEA tri-ethyl-amine
Temp. temperature
tert. tertiary
TFA trifluoroacetic acid
THF tetrahydrofuran
wt weight
X-Phos G1 Chloro-(2-dicyclohexylphosphino-2′,4′,6′-triisopropyl-1,1′ biphenyl)[2-(2-aminoethyl)-phenyl)]-palladium(II)

### Methods

### HPLC-MS Methods

### Method 1

### Method 2

| | |
|---|---|
| Method Name: | Z003_S05 |
| Device description: | Agilent 1200 with DA-and MS-Detector |
| Column: | XBridge C18_3.0 × 30 mm_2.5 μm |
| Column producer: | Waters |

Description:

| Gradient/ | % Sol | | | | |
|---|---|---|---|---|---|
| Solvent | [Water | | | | Back |
| Time | 0.1% | % Sol | Flow | Temp | pressure |
| [min] | NH₃] | [Acetonitrile] | [ml/min] | [° C.] | [PSI] |
| 0.0 | 95.0 | 5.0 | 2.2 | 60.0 | |
| 0.2 | 95.0 | 5.0 | 2.2 | 60.0 | |
| 1.2 | 0.0 | 100.0 | 2.2 | 60.0 | |
| 1.25 | 0.0 | 100.0 | 3.0 | 60.0 | |
| 1.4 | 0.0 | 100.0 | 3.0 | 60.0 | |

### Method 3

| | |
|---|---|
| Method Name: | Z011_S03 |
| Device description: | Agilent 1200 with DA-and MS-Detector |
| Column: | XBridge C18_3.0 × 30 mm_2.5 μm |
| Column producer: | Waters |

Description:

| Gradient/ | % Sol | | | | |
|---|---|---|---|---|---|
| Solvelnt | [Water | | | | Back |
| Time | 0.1% | % Sol | Flow | Temp | pressure |
| [min] | NH₃] | [Acetonitrile] | [ml/min] | [° C.] | [PSI] |
| 0.0 | 97.0 | 3.0 | 2.2 | 60.0 | |
| 0.2 | 97.0 | 3.0 | 2.2 | 60.0 | |
| 1.2 | 0.0 | 100.0 | 2.2 | 60.0 | |
| 1.25 | 0.0 | 100.0 | 3.0 | 60.0 | |
| 1.4 | 0.0 | 100.0 | 3.0 | 60.0 | |

### Method 4

| | |
|---|---|
| Method Name: | Z018_S04 |
| Device description: | Agilent 1200 with DA-and MS-Detector |
| Column: | Sunfire C18_3.0 × 30 mm_2.5 μm |
| Column producer: | Waters |

Description:

| Gradient/ | % Sol | | | | |
|---|---|---|---|---|---|
| Solvent | [Water | | | | Back |
| Time | 0.1% | % Sol | Flow | Temp | pressure |
| [min] | TFA] | [Acetonitrile] | [ml/min] | [° C.] | [PSI] |
| 0.0 | 97.0 | 3.0 | 2.2 | 60.0 | |
| 0.2 | 97.0 | 3.0 | 2.2 | 60.0 | |
| 1.2 | 0.0 | 100.0 | 2.2 | 60.0 | |
| 1.25 | 0.0 | 100.0 | 3.0 | 60.0 | |
| 1.4 | 0.0 | 100.0 | 3.0 | 60.0 | |

### Chiral SFC Analytical Methods:

### I_C2_10_M EGOH_NH3_002

| | |
|---|---|
| Method Name: | I_C2_10_MEOH_NH3_002 |
| Device description: | Agilent 1260 Infinity II SFC with DAD |
| Column: | Lux(R) Cellulose-2_3 × 100 mm_3 μm |
| Column producer: | Phenomenex |

Description:

| Gradient/ | | % Sol | | | |
|---|---|---|---|---|---|
| Solvent | | [MEOH | | | Back |
| Time | % Sol | 20 mM | Flow | Temp | pressure |
| [min] | [scCO2] | NH3] | [ml/min] | [° C.] | [PSI] |
| 0.0 | 90.0 | 10.0 | 2.0 | 40.0 | 2175.0 |
| 4.0 | 90.0 | 10.0 | 2.0 | 40.0 | 2175.0 |

### I_C2_20_MEOH_NH3_002

| | |
|---|---|
| Method Name: | I_C2_20_MEOH_NH3_002 |
| Device description: | Agilent 1260 Infinity II SFC with DAD |
| Column: | Lux(R) Cellulose-2_3 × 100 mm_3 μm |
| Column producer: | Phenomenex |

Description:

| Gradient/ | | % Sol | | | |
|---|---|---|---|---|---|
| Solvent | | [MEOH | | | Back |
| Time | % Sol | 20 mM | Flow | Temp | pressure |
| [min] | [scCO2] | NH3] | [ml/min] | [° C.] | [PSI] |
| 0.0 | 80.0 | 20.0 | 2.0 | 40.0 | 2175.0 |
| 4.0 | 80.0 | 20.0 | 2.0 | 40.0 | 2175.0 |

### I_C4_10_MEOH_NH3_002

| | |
|---|---|
| Method Name: | I_C4_10_MEOH_NH3_002 |
| Device description: | Agilent 1260 Infinity II SFC with DAD |
| Column: | Lux(R) Cellulose-4_3 × 100 mm_3 μm |
| Column producer: | Phenomenex |

Description:

| Gradient/ | | % Sol | | | |
|---|---|---|---|---|---|
| Solvent | | [MEOH | | | Back |
| Time | % Sol | 20 mM | Flow | Temp | pressure |
| [min] | [scCO2] | NH3] | [ml/min] | [° C.] | [PSI] |
| 0.0 | 90.0 | 10.0 | 2.0 | 40.0 | 2175.0 |
| 4.0 | 90.0 | 10.0 | 2.0 | 40.0 | 2175.0 |

### I_C4_15_MEOH_NH3_002

| | |
|---|---|
| Method Name: | I_C4_15_MEOH_NH3_002 |
| Device description: | Agilent 1260 Infinity II SFC with DAD |
| Column: | Lux(R) Cellulose-4_3 × 100 mm_3 μm |
| Column producer: | Phenomenex |

Description:

| Gradient/ | | % Sol | | | |
|---|---|---|---|---|---|
| Solvent | | [MEOH | | | Back |
| Time | % Sol | 20 mM | Flow | Temp | pressure |
| [min] | [scCO2] | NH3] | [ml/min] | [° C.] | [PSI] |
| 0.0 | 85.0 | 15.0 | 2.0 | 40.0 | 2175.0 |
| 4.0 | 85.0 | 15.0 | 2.0 | 40.0 | 2175.0 |

### I_C4_20_MEOH_NH3_001

| | |
|---|---|
| Method Name: | I_C4_20_MEOH_NH3_001 |
| Device description: | Agilent 1260 SFC with DAD and ELSD |
| Column: | Lux ® Cellulose-4_4.6 × 250 mm_5 μm |
| Column producer: | Phenomenex |

Description:

| Gradient/ | | % Sol | | | |
|---|---|---|---|---|---|
| Solvent | | [MEOH | | | Back |
| Time | % Sol | 20 mM | Flow | Temp | pressure |
| [min] | [scCO2] | NH3] | [ml/min] | [° C.] | [PSI] |
| 0.0 | 80.0 | 20.0 | 4.0 | 40.0 | 2175.0 |
| 10.0 | 80.0 | 20.0 | 4.0 | 40.0 | 2175.0 |

### I_IA_15_ETOH_NH3_001

| | |
|---|---|
| Method Name: | I_IA_15_ETOH_NH3_001 |
| Device description: | Agilent 1260 SFC with DAD and MS |
| Column: | Chiralpak ® IA_4.6 × 250 mm_5 um |
| Column producer: | Daicel |

Description:

| Gradient/ | | % Sol | | | |
|---|---|---|---|---|---|
| Solvent | | [ETOH | | | Back |
| Time | % Sol | 20 mM | Flow | Temp | pressure |
| [min] | [scCO2] | NH3] | [ml/min] | [° C.] | [PSI] |
| 0.0 | 85.0 | 15.0 | 4.0 | 40.0 | 2175.0 |
| 10.0 | 85.0 | 15.0 | 4.0 | 40.0 | 2175.0 |

### I_IG_10_MEOH_NH3_002

| | |
|---|---|
| Method Name: | I_IG_10_MEOH_NH3_002 |
| Device description: | Agilent 1260 Infinity II SFC with DAD |
| Column: | Chiralpak ® IG_3 × 100 mm_3 μm |
| Column producer: | Daicel |

Description:

| Gradient/ | | % Sol | | | |
|---|---|---|---|---|---|
| Solvent | | [MEOH | | | Back |
| Time | % Sol | 20 mM | Flow | Temp | pressure |
| [min] | [scCO2] | NH3] | [ml/min] | [° C.] | [PSI] |
| 0.0 | 90.0 | 10.0 | 2.0 | 40.0 | 2175.0 |
| 4.0 | 90.0 | 10.0 | 2.0 | 40.0 | 2175.0 |

### I_IG_15_IPA_NH3_001

| | |
|---|---|
| Method Name: | I_IG_15_IPA_NH3_001 |
| Device description: | Agilent 1260 SFC with DAD and MS |
| Column: | Chiralpak ® IG_4.6 × 250 mm_5 μm |
| Column producer: | Daicel |

Description:

| Gradient/ | | % Sol | | | |
|---|---|---|---|---|---|
| Solvent | | [IPA | | | Back |
| Time | % Sol | 20 mM | Flow | Temp | pressure |
| [min] | [scCO2] | NH3] | [ml/min] | [° C.] | [PSI] |
| 0.0 | 85.0 | 15.0 | 4.0 | 40.0 | 2175.0 |
| 10.0 | 85.0 | 15.0 | 4.0 | 40.0 | 2175.0 |

### I_IG_15_MEOH_NH3_001

| Method Name: | I_IG_15_MEOH_NH3_001 | | | | |
|---|---|---|---|---|---|
| Device description: | Agilent 1260 SFC with DAD and MS | | | | |
| Column: | Chiralpak ® IG_4.6 × 250 mm_5 μm | | | | |
| Column producer: | Daicel | | | | |
| Description: | | | | | |
| Gradient/Solvent Time | % Sol | % Sol [MEOH | Flow | Temp | Back pressure |
| [min] | [scCO2] | 20 mM NH3] | [ml/min] | [° C.] | [PSI] |
| 0.0 | 85.0 | 15.0 | 4.0 | 40.0 | 2175.0 |
| 10.0 | 85.0 | 15.0 | 4.0 | 40.0 | 2175.0 |

### I_IG_25_IPA_NH3_001

| Method Name: | I_IG_25_IPA_NH3_001 | | | | |
|---|---|---|---|---|---|
| Device description: | Agilent 1260 SFC with DAD and MS | | | | |
| Column: | Chiralpak ® IG_4.6 × 250 mm_5 μm | | | | |
| Column producer: | Daicel | | | | |
| Description: | | | | | |
| Gradient/Solvent Time | % Sol | % Sol [IPA | Flow | Temp | Back pressure |
| [min] | [scCO2] | 20 mM NH3] | [ml/min] | [° C.] | [PSI] |
| 0.0 | 75.0 | 25.0 | 4.0 | 40.0 | 2175.0 |
| 10.0 | 75.0 | 25.0 | 4.0 | 40.0 | 2175.0 |

### I_IG_25_MeOH_NH3_001

| Method Name: | I_IG_25_MEOH_NH3_001 | | | | |
|---|---|---|---|---|---|
| Device description: | Agilent 1260 SFC with DAD and MS | | | | |
| Column: | Chiralpak ® IG_4.6 × 250 mm_5 μm | | | | |
| Column producer: | Daicel | | | | |
| Description: | | | | | |
| Gradient/Solvent Time | % Sol | % Sol [MEOH | Flow | Temp | Back pressure |
| [min] | [scCO2] | 20 mM NH3] | [ml/min] | [° C.] | [PSI] |
| 0.0 | 75.0 | 25.0 | 4.0 | 40.0 | 2175.0 |
| 10.0 | 75.0 | 25.0 | 4.0 | 40.0 | 2175.0 |

### I_SA_10_IPA_NH3_001

| Method Name: | I_SA_10_IPA_NH3_001 | | | | |
|---|---|---|---|---|---|
| Device description: | Agilent 1260 SFC with DAD and MS | | | | |
| Column: | CHIRAL ART ® Amylose SA_4.6 × 250 mm_5 μm | | | | |
| Column producer: | YMC | | | | |
| Description: | | | | | |
| Gradient/Solvent Time | % Sol | % Sol [IPA | Flow | Temp | Back pressure |
| [min] | [scCO2] | 20mM NH3] | [ml/min] | [° C.] | [PSI] |
| 0.0 | 90.0 | 10.0 | 4.0 | 40.0 | 2175.0 |
| 10.0 | 90.0 | 10.0 | 4.0 | 40.0 | 2175.0 |

### I_SA_10_MEOH_NH3_001

| Method Name: | I_SA_10_MEOH_NH3_001 | | | | |
|---|---|---|---|---|---|
| Device description: | Agilent 1260 SFC with DAD and MS | | | | |
| Column: | CHIRAL ART ® Amylose SA_4.6 × 250 mm_5 μm | | | | |
| Column producer: | YMC | | | | |
| Description: | | | | | |
| Gradient/Solvent Time | % Sol | % Sol [MEOH | Flow | Temp | Back pressure |
| [min] | [scCO2] | 20 mM NH3] | [ml/min] | [° C.] | [PSI] |
| 0.0 | 90.0 | 10.0 | 4.0 | 40.0 | 2175.0 |
| 10.0 | 90.0 | 10.0 | 4.0 | 40.0 | 2175.0 |

### I_SA_15_MEOH_NH3_001

| Method Name: | I_SA_15_MEOH_NH3_001 | | | | |
|---|---|---|---|---|---|
| Device description: | Agilent 1260 SFC with DAD and MS | | | | |
| Column: | CHIRAL ART ® Amylose SA_4.6 × 250 mm_5 μm | | | | |
| Column producer: | YMC | | | | |
| Description: | | | | | |
| Gradient/Solvent Time | % Sol | % Sol [MEOH | Flow | Temp | Back pressure |
| [min] | [scCO2] | 20 mM NH3] | [ml/min] | [° C.] | [PSI] |
| 0.0 | 85.0 | 15.0 | 4.0 | 40.0 | 2175.0 |
| 10.0 | 85.0 | 15.0 | 4.0 | 40.0 | 2175.0 |

### I_SB_10_IPA_NH3_001

| Method Name: | I_SB_10_IPA_NH3_001 | | | | |
|---|---|---|---|---|---|
| Device description: | Agilent 1260 SFC with DAD and MS | | | | |
| Column: | CHIRAL ART ® Cellulose SB_4.6 × 250 mm_5 μm | | | | |
| Column producer: | YMC | | | | |
| Description: | | | | | |
| Gradient/Solvent Time | % Sol | % Sol [IPA | Flow | Temp | Back pressure |
| [min] | [scCO2] | 20 mM NH3] | [ml/min] | [° C.] | [PSI] |
| 0.0 | 90.0 | 10.0 | 4.0 | 40.0 | 2175.0 |
| 10.0 | 90.0 | 10.0 | 4.0 | 40.0 | 2175.0 |

### I_SB_20_MEOH_NH3_001

| Method Name: | I_SB_20_MEOH_NH3_001 | | | | |
|---|---|---|---|---|---|
| Device description: | Agilent 1260 SFC with DAD and MS | | | | |
| Column: | CHIRAL ART ® Cellulose SB_4.6 × 250 mm_5 μm | | | | |
| Column producer: | YMC | | | | |
| Description: | | | | | |
| Gradient/Solvent Time | % Sol | % Sol [MEOH | Flow | Temp | Back pressure |
| [min] | [scCO2] | 20 mM NH3] | [ml/min] | [° C.] | [PSI] |
| 0.0 | 80.0 | 20.0 | 4.0 | 40.0 | 2175.0 |
| 10.0 | 80.0 | 20.0 | 4.0 | 40.0 | 2175.0 |

### I_SC_05_IPA_NH3_001

| Method Name: | I_SC_05_IPA_NH3_001 | | | | |
|---|---|---|---|---|---|
| Device description: | Agilent 1260 SFC with DAD and MS | | | | |
| Column: | CHIRAL ART ® Cellulose SC_4.6 × 250 mm_5 μm | | | | |
| Column producer: | YMC | | | | |
| Description: | | | | | |
| Gradient/Solvent Time | % Sol | % Sol [IPA | Flow | Temp | Back pressure |
| [min] | [scCO2] | 20 mM NH3] | [ml/min] | [° C.] | [PSI] |
| 0.0 | 95.0 | 5.0 | 4.0 | 40.0 | 2175.0 |
| 10.0 | 95.0 | 5.0 | 4.0 | 40.0 | 2175.0 |

### I_SC_10_IPA_NH3_001

| Method Name: | I_SC_10_IPA_NH3_001 | | | | |
|---|---|---|---|---|---|
| Device description: | Agilent 1260 SFC with DAD and MS | | | | |
| Column: | CHIRAL ART ® Cellulose SC_4.6 × 250 mm_5 μm | | | | |
| Column producer: | YMC | | | | |
| Description: | | | | | |
| Gradient/Solvent Time | % Sol | % Sol [IPA | Flow | Temp | Back pressure |
| [min] | [scCO2] | 20 mM NH3] | [ml/min] | [° C.] | [PSI] |
| 0.0 | 90.0 | 10.0 | 4.0 | 40.0 | 2175.0 |
| 10.0 | 90.0 | 10.0 | 4.0 | 40.0 | 2175.0 |

### I_SC_10_MEOH_NH3_001

| Method Name: | I_SC_10_MEOH_NH3_001 | | | | |
|---|---|---|---|---|---|
| Device description: | Agilent 1260 SFC with DAD and MS | | | | |
| Column: | CHIRAL ART ® Cellulose SC_4.6 × 250 mm_5 μm | | | | |
| Column producer: | YMC | | | | |
| Description: | | | | | |
| Gradient/Solvent Time | % Sol | % Sol [MEOH | Flow | Temp | Back pressure |
| [min] | [scCO2] | 20 mM NH3] | [ml/min] | [° C.] | [PSI] |
| 0.0 | 90.0 | 10.0 | 4.0 | 40.0 | 2175.0 |
| 10.0 | 90.0 | 10.0 | 4.0 | 40.0 | 2175.0 |

### I_SC_15_IPA_NH3_001

| Method Name: | I_SC_15_IPA_NH3_001 | | | | |
|---|---|---|---|---|---|
| Device description: | Agilent 1260 SFC with DAD and MS | | | | |
| Column: | CHIRAL ART ® Cellulose SC_4.6 × 250 mm_5 μm | | | | |
| Column producer: | YMC | | | | |
| Description: | | | | | |
| Gradient/Solvent Time | % Sol | % Sol [IPA | Flow | Temp | Back pressure |
| [min] | [scCO2] | 20 mM NH3] | [ml/min] | [° C.] | [PSI] |
| 0.0 | 85.0 | 15.0 | 4.0 | 40.0 | 2175.0 |
| 10.0 | 85.0 | 15.0 | 4.0 | 40.0 | 2175.0 |

### I_SC_20_IPA_NH3_001

| Method Name: | I_SC_20_IPA_NH3_001 | | | | |
|---|---|---|---|---|---|
| Device description: | Agilent 1260 SFC with DAD and MS | | | | |
| Column: | CHIRAL ART ® Cellulose SC_4.6 × 250 mm_5 μm | | | | |
| Column producer: | YMC | | | | |
| Description: | | | | | |
| Gradient/Solvent Time | % Sol | % Sol [IPA | Flow | Temp | Back pressure |
| [min] | [scCO2] | 20 mM NH3] | [ml/min] | [° C.] | [PSI] |
| 0.0 | 80.0 | 20.0 | 4.0 | 40.0 | 2175.0 |
| 10.0 | 80.0 | 20.0 | 4.0 | 40.0 | 2175.0 |

### I_SC_20_MEOH_NH3_001

| Method Name: | I_SC_20_MEOH_NH3_001 | | | | |
|---|---|---|---|---|---|
| Device description: | Agilent 1260 SFC with DAD and MS | | | | |
| Column: | CHIRAL ART ® Cellulose SC_4.6 × 250 mm_5 μm | | | | |
| Column producer: | YMC | | | | |
| Description: | | | | | |
| Gradient/Solvent Time | % Sol | % Sol [IPA | Flow | Temp | Back pressure |
| [min] | [scCO2] | 20 mM NH3] | [ml/min] | [° C.] | [PSI] |
| 0.0 | 80.0 | 20.0 | 4.0 | 40.0 | 2175.0 |
| 10.0 | 80.0 | 20.0 | 4.0 | 40.0 | 2175.0 |

### I_SC_25_MEOH_NH3_001

| Method Name: | I_SC_25_MEOH_NH3_001 | | | | |
|---|---|---|---|---|---|
| Device description: | Agilent 1260 SFC with DAD and MS | | | | |
| Column: | CHIRAL ART ® Cellulose SC_4.6 × 250 mm_5 μm | | | | |
| Column producer: | YMC | | | | |
| Description: | | | | | |
| Gradient/Solvent Time | % Sol | % Sol [MEOH | Flow | Temp | Back pressure |
| [min] | [scCO2] | 20 mM NH3] | [ml/min] | [° C.] | [PSI] |
| 0.0 | 75.0 | 25.0 | 4.0 | 40.0 | 2175.0 |
| 10.0 | 75.0 | 25.0 | 4.0 | 40.0 | 2175.0 |

NMR method: NMR spectra were recorded on a Bruker AVANCE IIIHD 400 MHz instrument using TopSpin 3.2 pl6 software. Chemical shifts are given in parts per million (ppm) downfield from internal reference trimethylsilane in 6 units. Selected data are reported in the following manner: chemical shift (multiplicity, coupling constants (J), number of hydrogens). Abbreviations are as follows: s (singulet), d (doublet), t (triplet), q (quartet), spt (septet), m (multiplet), br (broad).

MS (ESI⁺): (M+H)⁺ 170

HPLC: RT=0.23 min, Method F

### EXAMPLES

### Example 1

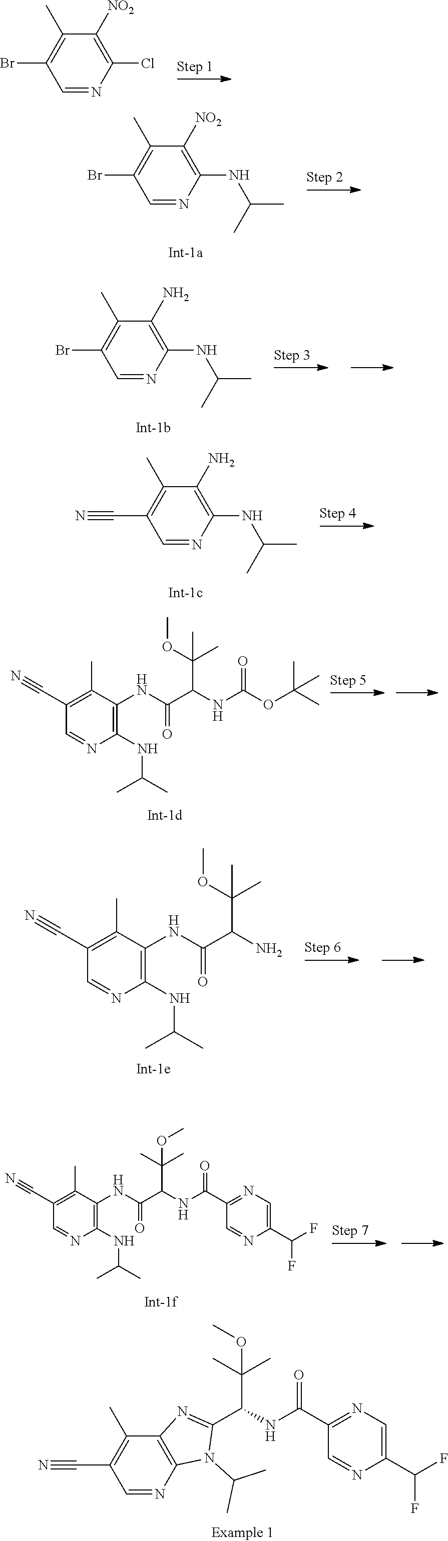

### Step 1:

5-Bromo-2-chloro-4-methyl-3-nitropyridine (2.0 g, 7.9 mmol) is mixed with isopropyl-amine (10 mL, 117 mmol) and stirred at ambient temperature for 16 h. The mixture is concentrated in vacuo. The residue is washed with water and filtrated, washed with water and dried i. vac.

Yield: 2.17 g (7.9 mmol; quant.) Int-1a

MS (ESI⁺): (M+H)⁺274/276 (Br); HPLC: RT=1.22 min, Method: Z018_S04

### Step 2:

Int-1a (1.0 g, 3.6 mmol) is mixed with 150 mg Raney nickel in 20 mL THF and hydrogenated at 50 psi at ambient temperature for 19 h. The mixture is filtrated, and the filtrate concentrated i. vac. Yield: 870 mg (3.6 mmol; 98%) Int-1b

MS (ESI⁺): (M+H)⁺244/246 (Br); HPLC: RT=0.94 min, Method: Z011_S03

### Step 3:

Int-1b (245 mg, 1.00 mmol) together with Zn(CN)₂ (200 mg, 1.70 mmol) and X-Phos G1 (70 mg, 0.10 mmol) in 2.0 mL NMP are stirred under argon at 110° C. for 16 h. Afterwards, ACN is added, the mixture filtrated and the filtrate is purified via prep. HPLC (C-18 X-Bridge at 50° C., eluent gradient (water+0.15% NH₃):ACN 79:21->59:41). The product containing fractions are combined and freeze-dried. Yield: 160 mg (0.84 mmol; 84%) Int-1c

MS (ESI⁺): (M+H)⁺191; HPLC: RT=0.61 min, Method: Z018_S04

### Step 4:

2-N-Boc-Amino-3-methoxy-3-methyl-butanoic acid (186 mg, 0.75 mmol) and Int-1c (130 mg, 0.68 mmol) in 1.5 mL pyridine are cooled to 0° C. and PPA (50% in EtOAc, 1.05 mL, 1.71 mmol) are added under stirring. After stirring for 2 h at ambient temperature, the mixture is concentrated i. vac. The residue is taken up with water and EtOAc 1:1 and extracted with EtOAc, the combined organic layers washed with sat. NaHCO₃ (aq.), dried over Na₂SO₄ and concentrated i. vac. The residue is treated with diethyl ether, filtrated and dried i. vac.

Yield: 260 mg (0.62 mmol; 91%) Int-1d

MS (ESI⁺): (M+H)⁺420; HPLC: RT=1.04 min, Method: Z011_S03

### Step 5:

Int-1d (270 mg, 0.64 mmol) is mixed with 5 mL 4 M HCl in dioxane and the mixture stirred at ambient temperature for 2 h. Afterwards, the mixture is concentrated i. vac.

Yield: 229 mg (0.64 mmol; quant.) Int-1^{e}. HPLC: RT=0.86 min, Method: Z011_S03

### Step 6:

To a mixture of 5-(difluoromethyl)pyrazine-2-carboxylic acid (109 mg, 0.63 mmol), TBTU (201 mg, 0.63 mmol) and TEA (0.40 mL, 2.85 mmol) in 3.0 mL DMF is added Int-1e (203 mg, 0.57 mmol) at ambient temperature and the mixture stirred for 3 h. 100 μL water is added the mixture purified by basic preparative HPLC. The fractions containing product are combined and freeze-dried.

Yield: 170 mg (0.36 mmol; 63%) Int-1f

MS (ESI⁺): (M+H)⁺476; HPLC: RT=1.01 min, Method: Z011_S03

### Step 7:

Int-1f (70 mg, 0.15 mmol) is stirred in 4.0 mL AcOH at 97° C. for 8d. Afterwards, the mixture is concentrated i. vac., taken up in THF/water, adjusted to basic pH by addition of NH₃ (aq.) and purified by basic preparative HPLC. The product containing fractions are unified and freeze-dried. The mixture is then separated by chiral SFC.

Yield: 34 mg (0.074 mmol; 49%) example 1

Example 1: N-[(1R)-1-[6-cyano-7-methyl-3-(propan-2-yl)-3H-imidazo[4,5-b]pyridin-2-
yl]-2-methoxy-2-methylpropyl]-5-(difluoromethyl)pyrazine-2-carboxamide

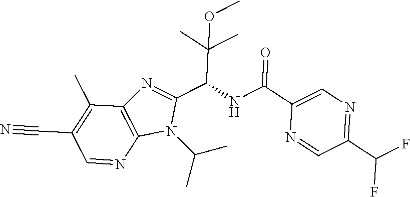 [SMILES: COC(C)(C)[C@H](NC(=O)c1cnc(C(F)F)cn1)c1nc2c(C)c(C#N)cnc2n1C(C)C]

| | |
|---|---|
| HPLC-MS; Method: Z011_S03; Rₜ [min]: 1.12 | MS: 458 (M + H)⁺ |
| Chiral SFC Rt Method I_C2_10_MEOH_NH3_002 | Rt [min]: 1.05 |

¹H NMR (400 MHz, DMSO-d₆) δ ppm 1.30 (s, 3 H) 1.35 (s, 3 H) 1.60-1.66 (m, 3 H)
1.69-1.75 (m, 3 H) 2.72 (s, 3 H) 3.18 (s, 3 H) 5.14 (quin, J = 6.75 Hz, 1 H) 5.61 (d,
J = 8.87 Hz, 1 H) 7.22 (m, 1 H) 8.67 (s, 1 H) 8.89 (d, J = 8.74 Hz, 1 H) 9.13 (s, 1 H) 9.28
(s, 1 H)

In analogy to example 1, the following products are obtained:

Example 2: N-[(IR)-1-[6-cyano-7-methyl-3-(propan-2-yl)-3H-imidazo[4,5-b]pyridin-2-
yl]-2-methoxy-2-methylpropyl]-5-methylpyrazine-2-carboxamide

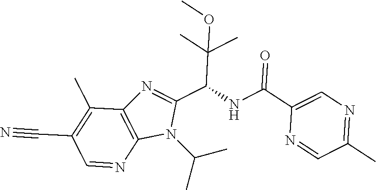 [SMILES: COC(C)(C)[C@H](NC(=O)c1cnc(C)cn1)c1nc2c(C)c(C#N)cnc2n1C(C)C]

| | |
|---|---|
| HPLC-MS; Method: Z011_S03; Rₜ [min]: 1.07 | MS: 422 (M + H)⁺ |
| Chiral SFC Rt Method: I_C2_20_MEOH_NH3_002 | Rt [min]: 1.08 |

¹H NMR (400 MHz, DMSO-d₆) δ ppm 1.28 (s, 3 H) 1.33 (s, 3 H) 1.59-1.65 (m, 3 H)
1.67-1.72 (m, 3 H) 2.61 (s, 3 H) 2.71 (s, 3 H) 3.18 (s, 3 H) 5.08-5.19 (m, 1 H) 5.58 (d,
J = 9.00 Hz, 1 H) 8.66 (s, 1 H) 8.70 (d, J = 1.01 Hz, 1 H) 8.78 (d, J = 8.87 Hz, 1 H) 9.05 (d,
J = 1.39 Hz, 1 H)

Example 3: N-[(S)-cyclopropyl[3-(propan-2-y1)-6-(trifluoromethyl)-3H-imidazo[4,5-
b]pyridin-2-y1]methyl]-5-methylpyrazine-2-carboxamide

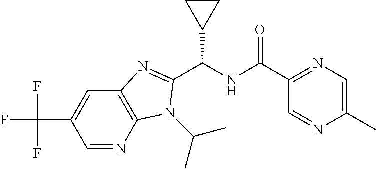 [SMILES: Cc1cnc(C(=O)N[C@H](c2nc3cc(C(F)(F)F)cnc3n2C(C)C)C2CC2)cn1]

| | |
|---|---|
| HPLC-MS; Method: Z018_S04; Rₜ [min]: 1.13 | MS: 419 (M + H)⁺ |
| Chiral SFC Rt Method: I_IG_10_MEOH_NH3_002 | Rt [min]: 1.22 |

¹H NMR (400 MHz, DMSO-d₆) δ ppm 0.47-0.67 (m, 4 H) 1.53-1.62 (m, 3 H) 1.67-
1.80 (m, 4 H) 2.60 (s, 3 H) 4.89-5.06 (m, 2 H) 8.50 (d, J = 1.65 Hz, 1 H) 8.65 (s, 1 H)
8.71 (d, J = 1.39 Hz, 1 H) 9.05 (d, J = 1.14 Hz, 1 H) 9.36 (d, J = 7.86 Hz, 1 H)

Example 4: N-[(S)-[6-cyano-7-methyl-3-(propan-2-yl)-3H-imidazo[4,5-b]pyridin-2-
yl](cyclopropyl)methyl]-5-(difluoromethyl)pyrazine-2-carboxamide

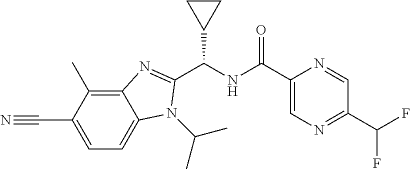 [SMILES: Cc1c(C#N)ccc2c1nc([C@@H](NC(=O)c1cnc(C(F)F)cn1)C1CC1)n2C(C)C]

| | |
|---|---|
| HPLC-MS; Method: Z011_S03; Rₜ [min]: 1.05 | MS: 426 (M + H)⁺ |
| Chiral SFC Rt Method: I_IG_15_MEOH_NH3_001 | Rt [min]: 1.99 |

¹H NMR (400 MHz, DMSO-d₆) δ ppm 0.51-0.66 (m, 4 H) 1.53-1.61 (m, 3 H) 1.65-
1.71 (m, 3 H) 1.72-1.82 (m, 1 H) 2.73 (s, 3 H) 4.91-5.00 (m, 2 H) 7.21 (m, 1 H) 8.66
(s, 1 H) 9.06 (s, 1 H) 9.27 (s, 1 H) 9.64 (br d, J = 7.60 Hz, 1 H)

Example 7: N-[(S)-[6-cyano-7-methyl-3-(propan-2-yl)-3H-
imidazo[4,5-b]pyridin-2-
yl](cyclobutyl)methyl]-5-(difluoromethyl)pyrazine-2-carboxamide

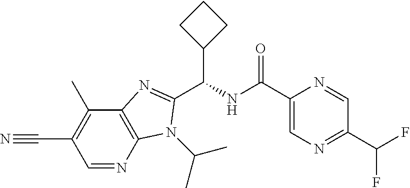 [SMILES: Cc1c(C#N)cnc2c1nc([C@@H](NC(=O)c1cnc(C(F)F)cn1)C1CCC1)n2C(C)C]

| | |
|---|---|
| HPLC-MS; Method: Z011_S03; Rₜ [min]: 1.12 | MS: 440 (M + H)⁺ |
| Chiral SFC Rt Method: I_SC_10_MEOH_NH3_001 | Rt [min]: 2.56 |

¹H NMR (400 MHz, DMSO-d₆) δ ppm 1.51-1.57 (m, 3 H) 1.66-1.75
(m, 3 H) 1.79-2.03 (m, 5 H) 2.04-2.13 (m, 1 H)
2.71 (s, 3 H) 3.32-3.37 (m, 1 H) 5.01 (quin, J = 6.75
Hz, 1 H) 5.57 (t, J = 8.93 Hz, 1 H) 7.20 (m, 1 H)
8.65 (s, 1 H) 9.03 (s, 1 H) 9.28 (d,
J = 1.27 Hz, 1 H) 9.43 (d, J = 8.24 Hz, 1 H)

Example 9: N-[(1R)-1-[6-chloro-3-(propan-2-yl)-3H-imidazo[4,5-b]pyridin-2-yl]-2-
methoxy-2-methylpropyl]-5-methylpyrazine-2-carboxamide

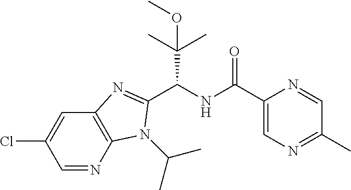 [SMILES: COC(C)(C)[C@H](NC(=O)c1cnc(C)cn1)c1nc2cc(Cl)cnc2n1C(C)C]

| | |
|---|---|
| HPLC-MS; Method: Z011_S03; Rₜ [min]: 1.13 | MS: 417 (M + H)⁺ |
| Chiral SFC Rt Method: I_C4_15_MEOH_NH3_002 | Rt [min]: 0.82 |

¹H NMR (400 MHz, DMSO-d₆) δ ppm 1.24 (s, 3 H) 1.32 (s, 3 H) 1.57-1.63 (m, 3 H)
1.70-1.75 (m, 3 H) 2.61 (s, 3 H) 3.18 (s, 3 H) 5.10 (quin, J = 6.78 Hz, 1 H) 5.55 (d,
J = 8.87 Hz, 1 H) 8.24 (d, J = 2.28 Hz, 1 H) 8.36 (d, J = 2.28 Hz, 1 H) 8.69 (d, J = 0.89 Hz, 1
H) 8.78 (br d, J = 8.4 Hz, 1 H) 9.06 (d, J = 1.27 Hz, 1 H)

Example 10: N-[(S)-[6-chloro-3-(propan-2-yl)-3H-imidazo[4,5-b]pyridin-2-
yl](cyclopropyl)methyl]-5-methylpyrazine-2-carboxamide

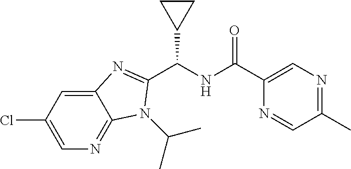 [SMILES: Cc1cnc(C(=O)N[C@H](c2nc3cc(Cl)cnc3n2C(C)C)C2CC2)cn1]

| | |
|---|---|
| HPLC-MS; Method: Z011_S03; Rₜ [min]: 1.05 | MS: 385 (M + H)⁺ |
| Chiral SFC Rt Method: I_IG_25_MEOH_NH3_001 | Rt [min]: 4.25 |

¹H NMR (400 MHz, DMSO-d₆) δ ppm 0.43-0.67 (m, 4 H) 1.49-1.60 (m, 3 H) 1.64-
1.77 (m, 4 H) 2.60 (s, 3 H) 4.84-4.95 (m, 1 H) 4.96-5.03 (m, 1 H) 8.22 (d, J = 2.28 Hz,
1 H) 8.35 (d, J = 2.28 Hz, 1 H) 8.64 (d, J = 1.14 Hz, 1 H) 9.05 (d, J = 1.39 Hz, 1 H) 9.29 (d,
J = 7.98 Hz, 1 H)

Example 12: N-[(1R,2R)-2-methoxy-1-[3-
(propan-2-yl)-6-(trifluoromethyl)-3H-imidazo
[4,5-b]pyridin-2-yl]propyl]-5-methylpyrazine-2-carboxamide

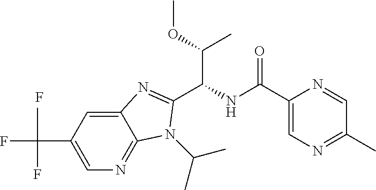

| | |
|---|---|
| HPLC-MS; Method: Z018_S04; Rₜ [min]: 1.14 | MS: 437 (M + H)⁺ |

¹H NMR (400 MHz, DMSO-d₆) δ ppm 1.10-1.18 (m, 3 H) 1.62-1.69
(m, 3 H) 1.72-1.79 (m, 3 H) 2.62 (s, 3 H) 3.30 (s, 3 H)
4.07 (quin, J = 5.92 Hz, 1 H) 5.11 (spt, J = 6.74
Hz, 1 H) 5.62 (dd, J = 7.92, 5.26 Hz, 1 H) 8.51 (d, J =
1.77 Hz, 1 H) 8.69 (s, 1 H) 8.73 (d, J = 1.39
Hz, 1 H) 9.00 (d, J = 7.86 Hz, 1 H) 9.08 (d, J = 1.27 Hz, 1 H)

Example 13: N-[(1R,2R)-1-[6-chloro-3-(propan-2-yl)-3H-imidazo[4,5-b]pyridin-2-yl]-2-
methoxypropyl]-5-methylpyrazine-2-carboxamide

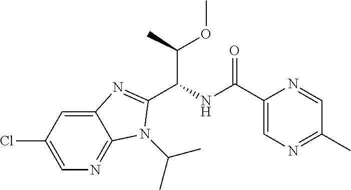 [SMILES: CO[C@H](C)[C@H](NC(=O)c1cnc(C)cn1)c1nc2cc(Cl)cnc2n1C(C)C]

| | |
|---|---|
| HPLC-MS; Method: Z018_S04; Rₜ [min]: 1.09 | MS: 403 (M + H)⁺ |
| Chiral SFC Rt Method: I_IG_15_MEOH_NH3_001 | Rt [min]: 3.72 |

¹H NMR (400 MHz, DMSO-d₆) δ ppm 1.07-1.13 (m, 3 H) 1.58-1.65 (m, 3 H) 1.69-1.74 (m, 3 H)
2.61 (s, 3 H) 3.30 (s, 3 H) 4.03 (quin, J = 5.96 Hz, 1 H) 4.96-5.10 (m, 1 H) 5.57 (dd, J = 7.98, 5.20
Hz, 1 H) 8.24 (d, J = 2.28 Hz, 1 H) 8.38 (d, J = 2.15 Hz, 1 H) 8.68 (d, J = 1.01 Hz, 1 H) 8.96 (d, J =
7.98 Hz, 1 H) 9.07 (d, J = 1.27 Hz, 1 H)

Example 15: N-[(S)-[6-cyano-7-methyl-3-(propan-2-yl)-3H-imidazo
[4,5-b]pyridin-2-yl](cyclobutyl)methyl]-5-methylpyrazine-2-carboxamide

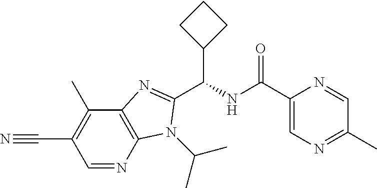 [SMILES: Cc1cnc(C(=O)N[C@H](c2nc3c(C)c(C#N)cnc3n2C(C)C)C2CCC2)cn1]

| | |
|---|---|
| HPLC-MS; Method: Z011_S03; Rₜ [min]: 1.07 | MS: 404 (M + H)⁺ |
| Chiral SFC Rt Method: I_SC_20_MEOH_NH3_001 | Rt [min]: 2.92 |

¹H NMR (400 MHz, DMSO-d₆) δ ppm 1.49-1.56 (m, 3 H)
1.65-1.73 (m, 3 H) 1.78-2.11 (m, 6 H) 2.59 (s, 3 H) 2.71
(s, 3 H) 3.23-3.35 (m, 1 H) 5.00 (quin, J = 6.78 Hz, 1 H)
5.55 (t, J = 8.93 Hz, 1 H) 8.62 (d, J = 1.01 Hz, 1 H) 8.64
(s, 1 H) 9.05 (d, J = 1.27 Hz, 1
H) 9.17 (d, J = 8.49 Hz, 1 H)

Example 17: N-[(R)-[6-cyano-7-methyl-3-(propan-2-yl)-3H-imidazo
[4,5-b]pyridin-2-yl][(2R)-oxolan-2-yl]methyl]-5-(difluoromethyl)
pyrazine-2-carboxamide

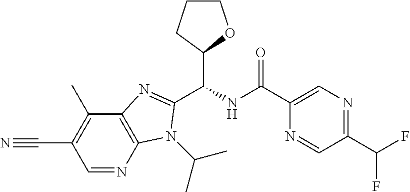 [SMILES: Cc1c(C#N)cnc2c1nc([C@@H](NC(=O)c1cnc(C(F)F)cn1)[C@H]1CCCO1)n2C(C)C]

| | |
|---|---|
| HPLC-MS; Method: Z011_S03; Rₜ [min]: 1.05 | MS: 456 (M + H)⁺ |
| Chiral SFC Rt Method: I_SB_10_IPA_NH3_001 | Rt [min]: 3.74 |

¹H NMR (400 MHz, DMSO-d₆) δ ppm 1.53-1.61 (m, 3 H)
1.64-1.90 (m, 6 H) 2.01-2.12 (m, 1 H) 2.73 (s, 3 H) 3.66-
3.83 (m, 2 H) 4.63 (q, J = 6.63 Hz, 1 H) 5.06 (spt, J = 6.70 Hz, 1 H)
5.61 (t, J = 7.41 Hz, 1 H) 7.22 (m, 1 H) 8.68 (s, 1 H) 9.09 (s, 1 H) 9.28
(d, J = 8.11 Hz, 1 H) 9.31 (s, 1 H)

Example 18: N-[(1S)-1-[6-cyano-7-methyl-3-(propan-2-yl)-3H-imidazo[4,5-b]pyridin-
2-yl]-2-[(2R)-oxolan-2-yl]ethyl]-5-methylpyrazine-2-carboxamide

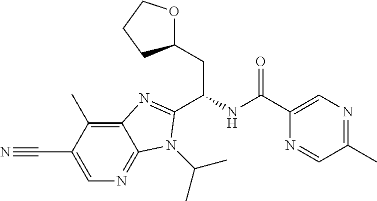 [SMILES: Cc1cnc(C(=O)N[C@@H](C[C@H]2CCCO2)c2nc3c(C)c(C#N)cnc3n2C(C)C)cn1]

| | |
|---|---|
| | MS: 434 (M + H)⁺ |
| Chiral SFC Rt Method: I_SC_25_MEOH_NH3_001 | Rt [min]: 3.01 |

¹H NMR (400 MHz, DMSO-d₆) δ ppm 1.44-1.55 (m, 1 H) 1.55-1.61 (m, 3 H) 1.66-
1.72 (m, 3 H) 1.72-1.90 (m, 2 H) 1.91-2.03 (m, 1 H) 2.16-2.27 (m, 1 H) 2.41-2.50
(m, 1 H) 2.58 (s, 3 H) 2.71 (s, 3 H) 3.52-3.63 (m, 1 H) 3.65-3.75 (m, 1 H) 3.77-3.83
(m, 1 H) 5.01 (dt, J = 13.50, 6.69 Hz, 1 H) 5.61 (q, J = 7.60 Hz, 1 H), 8.62 (s, 1 H) 8.66 (s,
1 H) 9.01-9.04 (m, 1 H) 9.34 (br d, J = 7.73 Hz, 1 H)

Example 19: N-[(1R,2R)-1-[6-cyano-7-methyl-3-(propan-2-yl)-3H-imidazo[4,5-
b]pyridin-2-yl]-2-methoxypropyl]-5-(difluoromethyl)pyrazine-2-carboxamide

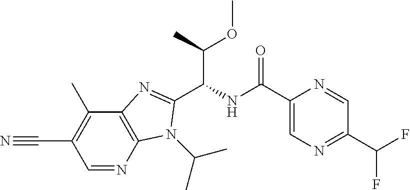 [SMILES: CO[C@H](C)[C@H](NC(=O)c1cnc(C(F)F)cn1)c1nc2c(C)c(C#N)cnc2n1C(C)C]

| | |
|---|---|
| HPLC-MS; Method: Z018_S04; Rₜ [min]: 1.11 | MS: 444 (M + H)⁺ |
| Chiral SFC Rt Method: I_IG_10_MEOH_NH3_002 | Rt [min]: 0.61 |

¹H NMR (400 MHz, DMSO-d₆) δ ppm 1.09-1.18 (m, 3 H) 1.58-1.66 (m, 3 H) 1.68-1.76 (m,
3 H) 2.72 (s, 3 H) 3.33 (s, 3 H) 4.12 (quin, J = 6.08 Hz, 1 H) 5.10 (spt, J = 6.65
Hz, 1 H) 5.57 (dd, J = 7.73, 5.96 Hz, 1 H) 7.22 (m, 1 H) 8.68 (s, 1 H) 9.10 (s, 1 H) 9.20
(d, J = 7.86 Hz, 1 H) 9.30 (s, 1 H)

Example 20: N-[(1R,2R)-1-[6-cyano-7-methyl-3-(propan-2-yl)-3H-imidazo[4,5-
b]pyridin-2-yl]-2-(difluoromethoxy)propyl]-5-(difluoromethyl)pyrazine-2-carboxamide

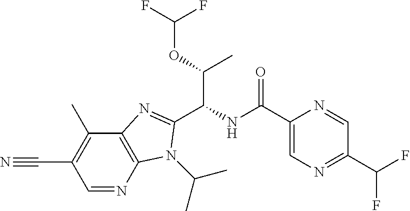 [SMILES: Cc1c(C#N)cnc2c1nc([C@@H](NC(=O)c1cnc(C(F)F)cn1)[C@@H](C)OC(F)F)n2C(C)C]

| | |
|---|---|
| HPLC-MS; Method: Z018_S04; Rₜ [min]: 1.13 | MS: 480 (M + H)⁺ |
| Chiral SFC Rt Method: I_SC_05_IPA_NH3_001 | Rt [min]: 3.19 |

¹H NMR (400 MHz, DMSO-d₆) δ ppm 1.31-1.39 (m, 3 H) 1.50-1.61 (m, 3 H) 1.68-1.78 (m, 3
H) 2.74 (s, 3 H) 4.99-5.10 (m, 2 H) 5.76 (dd, J = 8.36, 6.97 Hz, 1 H) 6.86 (m,
1 H) 7.21 (m, 1 H) 8.70 (s, 1 H) 9.08 (s, 1 H) 9.31 (s, 1 H) 9.43 (d, J = 8.62 Hz, 1 H)

Example 21: N-[(1R,2R)-1-[6-cyano-7-methyl-3-(propan-2-yl)-3H-imidazo[4,5-
b]pyridin-2-yl]-2-(difluoromethoxy)propyl]-5-methylpyrazine-2-carboxamide

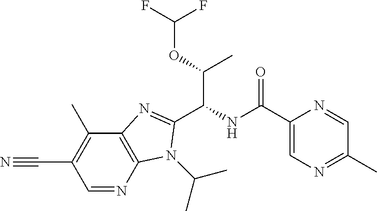 [SMILES: Cc1cnc(C(=O)N[C@H](c2nc3c(C)c(C#N)cnc3n2C(C)C)[C@@H](C)OC(F)F)cn1]

| | |
|---|---|
| HPLC-MS; Method: Z018_S04; Rₜ [min]: 1.10 | MS: 444 (M + H)⁺ |
| Chiral SFC Rt Method: I_SC_15_IPA_NH3_001 | Rt [min]: 2.39 |

¹H NMR (400 MHz, DMSO-d₆) δ ppm 1.30-1.37 (m, 3 H) 1.51-1.58 (m, 3 H) 1.68-1.76 (m,
3 H) 2.60 (s, 3 H) 2.73 (s, 3 H) 4.97-5.09 (m, 2 H) 5.73 (dd, J = 8.68, 6.65 Hz,
1 H) 6.86 (m, 1 H) 8.66 (d, J = 1.01 Hz, 1 H) 8.69 (s, 1 H) 9.08 (d, J = 1.39 Hz, 1 H) 9.19
(d, J = 8.74 Hz, 1 H)

Example 23: N-[(1R,2S)-1-[6-chloro-3-(propan-2-yl)-3H-imidazo[4,5-b]pyridin-2-yl]-2-
methoxypropyl]-5-methylpyrazine-2-carboxamide

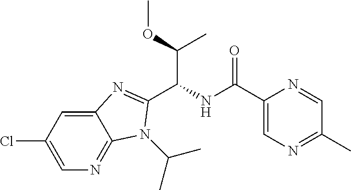 [SMILES: CO[C@@H](C)[C@H](NC(=O)c1cnc(C)cn1)c1nc2cc(Cl)cnc2n1C(C)C]

| | |
|---|---|
| HPLC-MS; Method: Z018_S04; Rₜ [min]: 1.08 | MS: 403 (M + H)⁺ |
| Chiral SFC Rt Method: I_C4_10_MEOH_NH3_002 | Rt [min]: 0.92 |

¹H NMR (400 MHz, DMSO-d₆) δ ppm 1.26 (d, J = 6.08 Hz, 3 H) 1.61 (d, J = 6.84 Hz, 3 H)
1.70 (d, J = 6.84 Hz, 3 H) 2.60 (s, 3 H) 3.16 (s, 3 H) 3.97-4.06 (m, 1 H) 4.98 (quin,
J = 6.72 Hz, 1 H) 5.45 (m, 1 H) 8.20 (d, J = 2.28 Hz, 1 H) 8.36 (d, J = 2.28 Hz, 1 H) 8.66
(s, 1 H) 9.04 (d, J = 1.27 Hz, 1 H) 9.18 (d, J = 8.74 Hz, 1 H)

Example 24: 5-(difluoromethyl)-N-[(1R)-2-methoxy-1-[3-(propan-2-yl)-6-
(trifluoromethyl)-3H-imidazo[4,5-b]pyridin-2-yl]propyl]pyrazine-2-carboxamide

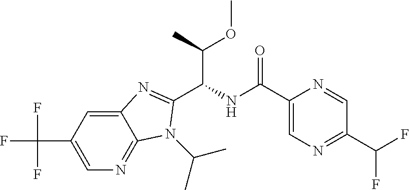 [SMILES: CO[C@H](C)[C@H](NC(=O)c1cnc(C(F)F)cn1)c1nc2cc(C(F)(F)F)cnc2n1C(C)C]

| | |
|---|---|
| HPLC-MS; Method: Z018_S04; Rₜ [min]: 1.19 | MS: 473 (M + H)⁺; |

¹H NMR (400 MHz, DMSO-d₆) δ ppm 1.11-1.16 (m, 3 H) 1.63-1.70 (m, 3 H) 1.73-1.79 (m,
3 H) 3.32 (s, 3 H) 4.06-4.13 (m, 1 H) 5.04-5.17 (m, 1 H) 5.64 (dd, J = 7.86, 5.45 Hz, 1 H)
7.23 (m, 1 H) 8.51 (d, J = 1.77 Hz, 1 H) 8.73-8.75 (m, 1 H) 9.11 (s, 1 H) 9.17 (d, J = 7.86
Hz, 1 H) 9.31 (d, J = 1.14 Hz, 1 H)

Example 26: N-[(R)-[6-cyano-7-methyl-3-(propan-2-yl)-3H-imidazo[4,5-b]pyridin-
2-yl](1-hydroxycyclobutyl)methyl]-5-(difluoromethyl)pyridazine-2-carboxamide

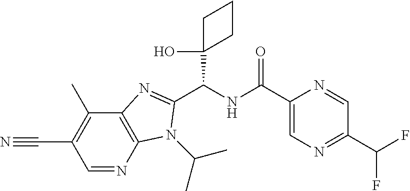 [SMILES: Cc1c(C#N)cnc2c1nc([C@@H](NC(=O)c1cnc(C(F)F)cn1)C1(O)CCC1)n2C(C)C]

| | |
|---|---|
| HPLC-MS; Method: Z011_S03; Rₜ [min]: 1.08 | MS: 456 (M + H)⁺ |
| Chiral SFC Rt Method: | Rt [min]: 3.19 |
| I_SB_20_MEOH_NH3_001 | |

¹H NMR (400 MHz, DMSO-d₆) δ ppm 1.56-1.62 (m, 3 H) 1.64-1.73 (m, 4 H) 1.74-1.88 (m,
1 H) 1.96-2.12 (m, 2 H) 2.23-2.33 (m, 1 H) 2.34-2.44 (m, 1 H) 2.73 (s, 3 H) 5.13 (spt, J = 6.74
Hz, 1 H) 5.64-5.69 (m, 1 H) 5.87 (s, 1 H) 7.21 (m, 1 H) 8.67 (s, 1 H) 9.00 (d, J = 8.49 Hz, 1
H) 9.11 (s, 1 H) 9.30-9.33 (m, 1 H)

Example 30: N-[(S)-[6-cyano-3-(propan-2-yl)-3H-imidazo[4,5-b]pyridin-2-
yl](cyclobutyl)methyl]-5-(difluoromethyl)pyrazine-2-carboxamide

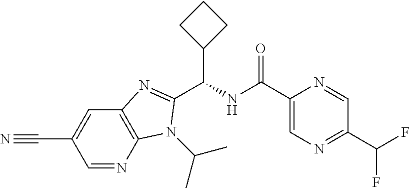 [SMILES: CC(C)n1c([C@@H](NC(=O)c2cnc(C(F)F)cn2)C2CCC2)nc2cc(C#N)cnc21]

| | |
|---|---|
| HPLC-MS; Method: Z011_S03; Rₜ [min]: 1.08 | MS: 426 (M + H)⁺ |
| Chiral SFC Rt Method: I_SC_10_MEOH_NH3_001 | Rt [min]: 2.31 |

¹H NMR (400 MHz, DMSO-d₆) δ ppm 1.48-1.57 (m, 3 H) 1.67-1.77 (m, 3 H) 1.79-
2.13 (m, 6 H) 3.22-3.34 (m, 1 H) 4.99 (spt, J = 6.74 Hz, 1 H) 5.61 (t, J = 8.87 Hz, 1 H)
7.20 (m, 1 H) 8.63 (d, J = 1.90 Hz, 1 H) 8.74 (d, J = 1.90 Hz, 1 H) 9.03 (s, 1 H) 9.30 (s, 1 H)
9.44 (d, J = 8.49 Hz, 1 H)

| Example 34: N-[(R)-[6-chloro-3-(propan-2-yl)-3H-imidazo[4,5-b] | |
|---|---|
| pyridin-2-yl][(2R)-oxolan-2-yl]methyl]-5-methylpyrazine- | |
| 2-carboxamide | |
| 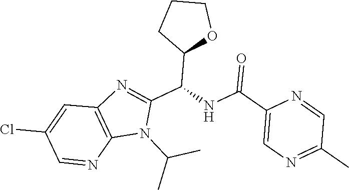 [SMILES: Cc1cnc(C(=O)N[C@H](c2nc3cc(Cl)cnc3n2C(C)C)[C@H]2CCCO2)cn1] | |
| HPLC-MS; Method: | MS: 415 (M + H)⁺ |
| Z011_S03; Rₜ [min]: 1.05 | |
| Chiral SFC Rt Method: | Rt [min]: 3.34 |
| I_ IG_25_MEOH_NH3_001 | |

| Example 37: N-[(1R)-2-methoxy-2-methyl-1-[3-(propan-2-yl)- | |
|---|---|
| 3H-imidazo[4,5-b]pyridin-2-yl]propyl]-5-methylpyrazine- | |
| 2-carboxamide | |
| 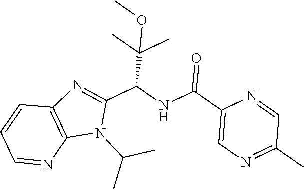 [SMILES: COC(C)(C)[C@H](NC(=O)c1cnc(C)cn1)c1nc2cccnc2n1C(C)C] | |
| HPLC-MS; Method: | MS: 383 (M + H)⁺ |
| Z011_S03; Rₜ [min]: 1.00 | |
| Chiral SFC Rt Method: | Rt [min]: 1.84 |
| I_C4_20_MEOH_NH3_001 | |

¹H NMR (400 MHz, DMSO-d₆) δ ppm 1.24 (s, 3 H) 1.33 (s,
3 H) 1.59-1.66 (m, 3 H) 1.72-1.79 (m, 3 H) 2.60
(s, 3 H) 3.18 (s, 3 H) 5.08 (quin, J = 6.81 Hz, 1 H) 5.53-5.59
(m, 1 H) 7.24 (dd, J = 8.05, 4.75 Hz, 1 H) 8.04 (dd,
J = 8.05, 1.46 Hz, 1 H) 8.32 (dd, J = 4.75, 1.46 Hz, 1 H) 8.69
(d, J = 1.01 Hz, 1 H) 8.79 (d, J = 9.00 Hz, 1 H) 9.06
(d, J = 1.27 Hz, 1 H)

Example 38: N-[(1S)-1-[6-cyano-7-methyl-3-(propan-2-yl)-3H-imidazo[4,5-b]pyridin-
2-yl]-2-[(2S)-oxolan-2-yl]ethyl]-5-methylpyrazine-2-carboxamide

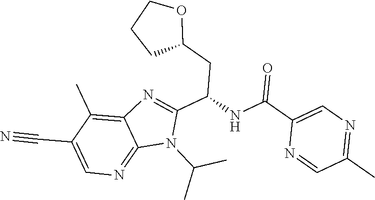 [SMILES: Cc1cnc(C(=O)N[C@@H](C[C@@H]2CCCO2)c2nc3c(C)c(C#N)cnc3n2C(C)C)cn1]

| | |
|---|---|
| | MS: 434 (M + H)⁺ |
| Chiral SFC Rt Method: I_SC_20_MEOH_NH3_001 | Rt [min]: 3.78 |

¹H NMR (400 MHz, DMSO-d₆) δ ppm 1.42-1.54 (m, 1 H) 1.54-1.62 (m, 3 H) 1.65-1.72 (m,
3 H) 1.73-1.89 (m, 2 H) 1.90-2.01 (m, 1 H) 2.07-2.18 (m, 1 H) 2.35-2.47
(m, 1 H) 2.59 (s, 3 H) 2.69 (m, 3 H) 3.57-3.66 (m, 1 H) 3.70-3.79 (m, 1 H) 3.92-
4.00 (m, 1 H) 5.00 (spt, J = 6.76 Hz, 1 H) 5.65 (td, J = 8.68, 3.80 Hz, 1 H) 8.64 (s, 2 H)
9.03 (d, J = 1.27 Hz, 1 H) 9.41 (d, J = 7.98 Hz, 1 H)

Example 41: N-[(R)-[6-chloro-3-(propan-2-yl)-3H-imidazo[4,5-b]pyridin-2-yl][(2S)-
oxolan-2-yl]methyl]-5-methylpyrazine-2-carboxamide

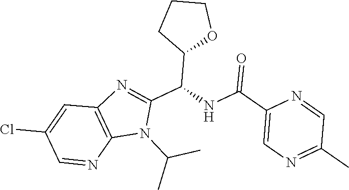 [SMILES: Cc1cnc(C(=O)N[C@H](c2nc3cc(Cl)cnc3n2C(C)C)[C@@H]2CCCO2)cn1]

| | |
|---|---|
| HPLC-MS; Method: Z018_S04; Rₜ [min]: 1.06 | MS: 415 (M + H)⁺ |
| Chiral SFC Rt Method: I_IG_25_MEOH_NH3_001 | Rt [min]: 5.31 |

¹H NMR (400 MHz, DMSO-d₆) δ ppm 1.58-1.66 (m, 3 H) 1.68-1.74 (m, 3 H) 1.80-
2.02 (m, 3 H) 2.06-2.18 (m, 1 H) 2.60 (m, 3 H) 3.62-3.70 (m 1 H) 3.80-3.88 (m, 1
H) 4.48 (ddd, J = 8.52, 6.88, 5.51 Hz, 1 H) 4.99 (spt, J = 6.74 Hz, 1 H) 5.41 (t, J = 8.55 Hz,
1 H) 8.18 (d, J = 2.28 Hz, 1 H) 8.35 (d, J = 2.15 Hz, 1 H) 8.66 (d, J = 1.01 Hz, 1 H) 9.04 (d,
J = 1.27 Hz, 1 H) 9.21 (d, J = 8.49 Hz, 1 H)

Example 42: N-[(1R,2R)-1-[6-cyano-7-methyl-3-(propan-2-yl)-3H-imidazo[4,5-
b]pyridin-2-yl]-2-methoxypropyl]-5-methylpyrazine-2-carboxamide

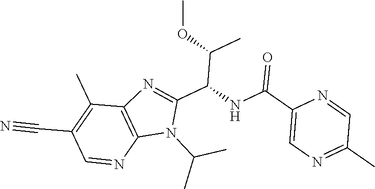 [SMILES: CO[C@H](C)[C@H](NC(=O)c1cnc(C)cn1)c1nc2c(C)c(C#N)cnc2n1C(C)C]

| | |
|---|---|
| HPLC-MS; Method: Z018_S04; Rₜ [min]: 1.06 | MS: 408 (M + H)⁺ |
| Chiral SFC Rt Method: I_C2_20_MEOH_NH3_002 | Rt [min]: 1.21 |

¹H NMR (400 MHz, DMSO-d₆) δ ppm 1.13 (d, J = 6.08 Hz, 3 H) 1.62 (d, J = 6.72 Hz, 3 H)
1.71 (d, J = 6.72 Hz, 3 H) 2.61 (s, 3 H) 2.71 (s, 3 H) 3.32 (s, 3 H) 4.09 (quin, J = 6.05
Hz, 1 H) 5.08 (spt, J = 6.70 Hz, 1 H) 5.55 (dd, J = 7.86, 5.58 Hz, 1 H) 8.67 (s, 1 H) 8.68
(d, J = 1.01 Hz, 1 H) 9.00 (d, J = 7.86 Hz, 1 H) 9.06 (d, J = 1.39 Hz, 1 H)

Example 45: N-[(S)-cyclopropyl[1-ethyl-4-methyl-6-(trifluoromethyl)-1H-imidazo[4,5-
c]pyridin-2-yl]methyl]-5-methylpyrazine-2-carboxamide

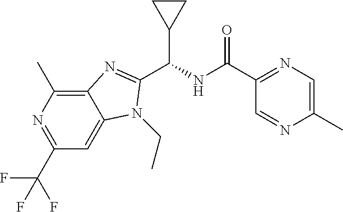 [SMILES: CCn1c([C@@H](NC(=O)c2cnc(C)cn2)C2CC2)nc2c(C)nc(C(F)(F)F)cc21]

| | |
|---|---|
| HPLC-MS; Method: Z018_S04; Rₜ [min]: 1.01 | MS: 419 (M + H)⁺ |
| Chiral SFC Rt Method: I_SA_15_MEOH_NH3_001 | Rt [min]: 1.57 |

¹H NMR (400 MHz, DMSO-d₆) δ ppm 0.43-0.69 (m, 4 H) 1.23-1.35 (m, 3 H) 1.77-1.87 (m,
1 H) 2.60 (s, 3 H) 2.77 (s, 3 H) 4.36-4.56 (m, 2 H) 4.96 (t, J = 8.49 Hz, 1 H) 8.09 (s, 1 H) 8.65
(s, 1 H) 9.04 (s, 1 H) 9.32 (br d, J = 7.98 Hz, 1 H)

Example 50: N-[(R)-[(2R)-1,4-dioxan-2-yl][1-(propan-2-yl)-6-(trifluoromethyl)-1H-
imidazo[4,5-c]pyridin-2-yl]methyl]-5-methylpyrazine-2-carboxamide

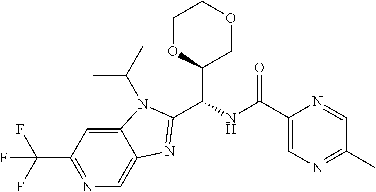 [SMILES: Cc1cnc(C(=O)N[C@H](c2nc3cnc(C(F)(F)F)cc3n2C(C)C)[C@@H]2COCCO2)cn1]

| | |
|---|---|
| HPLC-MS; Method: Z011_S03; Rₜ [min]: 0.94 | MS: 465 (M + H)⁺ |
| Chiral SFC Rt Method: I_SB_10_IPA_NH3_001 | Rt [min]: 2.98 |

¹H NMR (400 MHz, DMSO-d₆) δ ppm 1.54 (d, J = 6.84 Hz, 3 H) 1.66 (d, J = 6.84 Hz, 3
H) 2.61 (s, 3 H) 3.32-3.39 (m, 1 H) 3.41-3.53 (m, 1 H) 3.57-3.69 (m, 2 H) 3.78-3.92 (m, 2 H)
4.26-4.36 (m, 1 H) 5.14 (spt, J = 6.82 Hz, 1 H) 5.68-5.77 (m, 1 H) 8.26 (s, 1 H) 8.68
(d, J = 0.89 Hz, 1 H) 9.07 (d, J = 1.27 Hz, 1 H) 9.10 (s, 1 H) 9.16 (d, J = 8.24 Hz, 1 H)

### Example 5

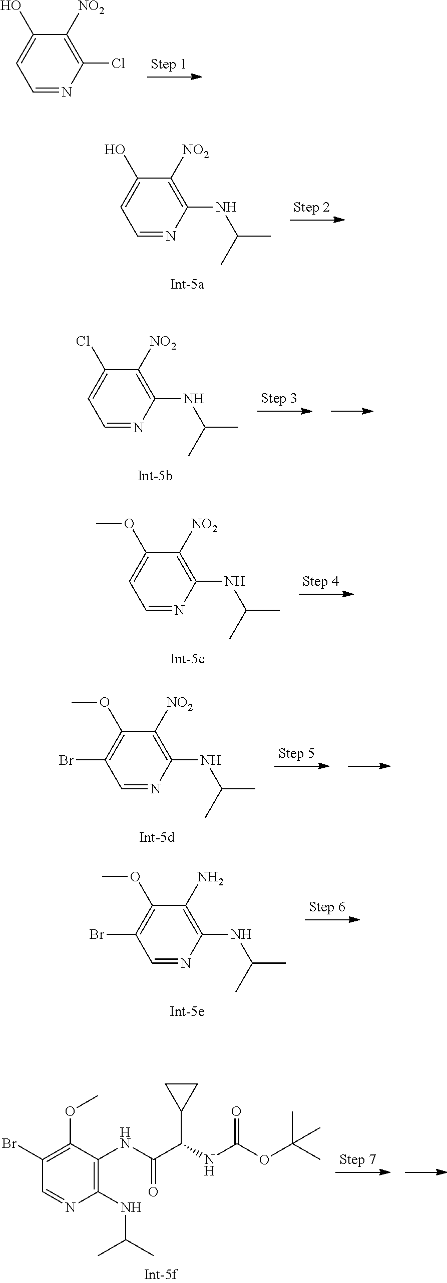

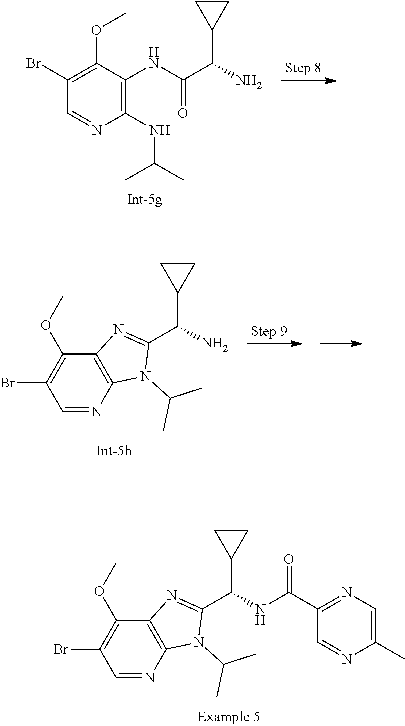 [SMILES: COc1c(Br)cnc(NC(C)C)c1NC(=O)[C@@H](N)C1CC1 ; COc1c(Br)cnc2c1nc([C@@H](N)C1CC1)n2C(C)C ; COc1c(Br)cnc2c1nc([C@@H](NC(=O)c1cnc(C)cn1)C1CC1)n2C(C)C]

### Step 1:

A mixture of 2-chloro-4-hydroxy-3-nitro-pyridine (2.00 g; 11.5 mmol) and isopropylamine (3.6 mL, 41.9 mmol) in 20 mL n-butanol are stirred in an autoclave at 110° C. for 18 h. The mixture is concentrated i. vac., taken up in MeOH and acidified with AcOH. The mixture is filtrated. The filtrate is purified by column chromatography (C18 Sunfire, 50° C., eluent gradient: (H₂O+0.1% TFA):ACN 95:5->75:25). Product containing fractions are combined and freeze-dried. The lyophilizate is combined with the solid from the filtration and dried i. vac. Yield: 1.59 g (8.06 mmol; 70%) Int-5a. MS (ESI⁺): (M+H)⁺198; HPLC: RT=0.61 min, Method: Z018_S04

### Step 2:

A mixture of Int-5a (1.59 g, 8.06 mmol) and POCl₃ (2 mL, 21 mmol) in 20 mL ACN is stirred for min at 80° C. Afterwards, the mixture is concentrated i. vac., the residue taken up with DCM and water, the mixture basified with 2N Na₂CO₃ (aq.) and the aqueous phase extracted with DCM. The combined organic layers are dried over MgSO₄, concentrated i. vac., and the residue is used without further purification. Yield: 1.74 g (8.07 mmol; quant.) Int-5b

MS (ESI⁺): (M+H)⁺216/218 (Cl); HPLC: RT=1.13 min, Method: Z018_S04

### Step 3:

A mixture of Int-5b (1.74 g, 8.07 mmol) and sodium methoxide (5.4N in MeOH, 2.25 mL, 12.2 mmol) in 15 mL MeOH and 15 mL THF are stirred for 3d at ambient temperature. Then the mixture is concentrated i. vac., the residue treated with water and filtrated. The solid is washed with ACN and dried i. vac. Yield: 1.62 g (7.67 mmol; 95%) Int-5c MS (ESI⁺): (M+H)⁺212; HPLC: RT=0.84 min, Method: Z018_S04

### Step 4:

A mixture of Int-5c (1.62 g, 7.67 mmol) and NBS (1.40 g, 7.87 mmol) in 30 mL ACN is stirred for 6 h at ambient temperature and then for 20 min at 45° C. Then the mixture is concentrated i. vac., the residue taken up with 0.5N Na₂CO₃ and DCM, the aq. phase extracted with DCM, the combined organic layers dried over MgSO₄ and concentrated i. vac. Yield: 2.23 g (7.67 mmol; quant.) Int-5d. MS (ESI⁺): (M+H)⁺290/292 (Br); HPLC: RT=1.18 min, Method: Z018_S04

### Step 5:

Int-5d (400 mg, 1.38 mmol) is mixed with Raney-nickel (100 mg) in 20 mL THF and hydrogenated for 20 h at 50 psi hydrogen pressure. Then the mixture is filtered and concentrated i. vac.

Yield: 350 mg (1.35 mmol; 98%) Int-5e. MS (ESI⁺): (M+H)⁺260/262 (Br); HPLC: RT=0.65 min, Method: Z018_S04

### Step 6:

To Int-5e (350 mg, 1.35 mmol) and (2S)-2-Boc-amino-2-cyclopropyl-acetic acid (300 mg, 1.39 mmol) in 5 mL pyridine is added PPA (50 wt % in AcOH, 1.5 mL, 2.45 mmol) at 0° C. and then stirred at 0° C. for 1 h. Water and ACN are added and the mixture purified by column chromatography (XBridge C18, 50° C., eluent gradient: (H₂O+0.1% NH₃):ACN 55:45->35:65).

Product containing fractions are combined and freeze-dried.

Yield: 440 mg (0.96 mmol; 72%) Int-5f

MS (ESI⁺): (M+H)⁺457/459 (Br); HPLC: RT=0.92 min, Method: Z018_S04

### Step 7:

Int-5f (440 mg, 0.96 mmol) in HCl in dioxane (4M, 4 mL) is stirred at ambient temperature for 1 h. The mixture is concentrated i. vac. and the residue taken up in 1n Na₂CO₃ (aq.) and DCM. The aq. phase is extracted with DCM, the organic layers combined, dried over MgSO₄ and concentrated i. vac.

Yield: 310 mg (0.87 mmol; 90%) Int-5 g

MS (ESI⁺): (M+H)⁺357/359 (Br); HPLC: RT=0.66 min, Method: Z018_S04

### Step 8:

Int-5 g (310 mg, 0.26 mol) and ZnBr₂ (400 mg, 1.78 mmol) in 5 mL n-butyl acetate are stirred at 100° C. for 3 h and then stirred at ambient temperature for 16 h. Afterwards, the mixture is concentrated i. vac., the residue taken up with ACN, acidified by addition of AcOH and some water added and filtrated. The filtrate is purified by column chromatography (Sunfire C-18, 50° C., eluent gradient: (H₂O+0.15% TFA):ACN 80:20->60:40). Product containing fractions are combined and freeze-dried.

Yield: 230 mg (0.51 mmol; 58%) Int-5 h

MS (ESI⁺): (M+H)⁺339/341 (Br); HPLC: RT=0.83 min, Method: Z018_S04

### Step 9:

To a mixture of Int-5 h (230 mg, 0.51 mmol), 5-methyl-pyrazine-carboxylic acid (100 mg, 0.72 mmol) and TEA (400 mg, 3.95 mmol) in 4 mL DMF is added TBTU (180 mg, 0.56 mmol) and the mixture stirred at ambient temperature for 15 min. Water is added and the mixture is purified by column chromatography (XBridge C18, 50° C., eluent gradient: (H₂O+0.15% NH₃):ACN 46:54->26:74). Product containing fractions are combined and freeze-dried.

Yield: 210 mg (0.46 mmol; 90%) example 5

Example 5: N-[(S)-[6-bromo-7-methoxy-3-(propan-2-yl)-3H-imidazo[4,5-b]pyridin-2-
yl](cyclopropyl)methyl]-5-methylpyrazine-2-carboxamide

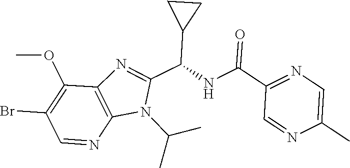 [SMILES: COc1c(Br)cnc2c1nc([C@@H](NC(=O)c1cnc(C)cn1)C1CC1)n2C(C)C]

| | |
|---|---|
| HPLC-MS; Method: Z018_S04; Rₜ [min]: 1.21 | MS: 459 (M + H)⁺ |
| Chiral SFC Rt Method: Method I_SC_20_MEOH_NH3_001 | Rt [min]: 2.83 |

¹H NMR (400 MHz, DMSO-d₆) δ ppm 0.00 (s, 1 H) 0.43-0.64 (m, 4 H) 1.47-1.57 (m,
3 H) 1.61-1.69 (m, 3 H) 1.69-1.78 (m, 1 H) 2.60 (s, 3 H) 4.59 (s, 3 H) 4.83-4.99 (m,
2 H) 8.30 (s, 1 H) 8.64 (d, J = 1.01 Hz, 1 H) 9.06 (d, J = 1.27 Hz, 1 H) 9.27 (d, J = 7.98 Hz,
1 H)

### Example 6

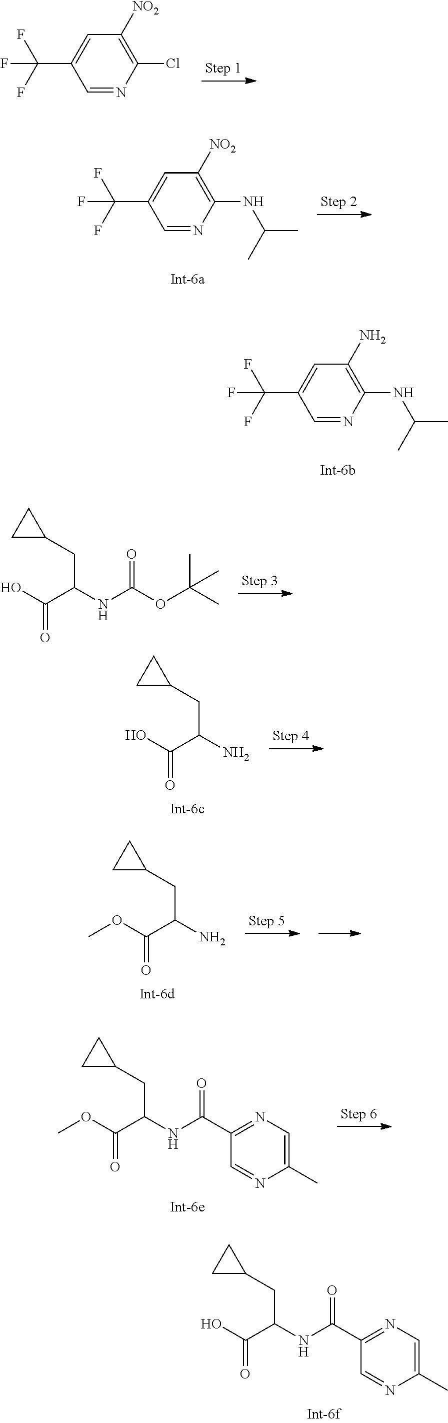

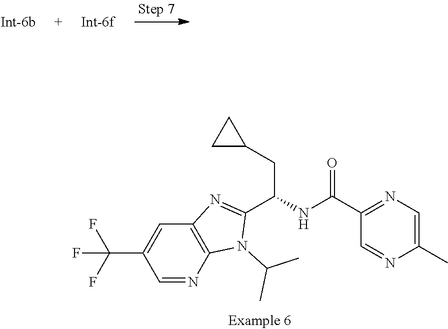 [SMILES: Cc1cnc(C(=O)N[C@@H](CC2CC2)c2nc3cc(C(F)(F)F)cnc3n2C(C)C)cn1]

### Step 1:

A mixture of 2-chloro-3-nitro-5-trifluoromethyl-pyridine (1.50 g; 6.62 mmol) and isopropylamine (2.0 mL, 23.5 mmol) in 10 mL THF are stirred at ambient temperature for 10 min. The mixture is concentrated i. vac., the residue treated with water, filtrated and the solid washed with water and dried i. vac. Yield: 1.55 g (6.22 mmol; 94%) Int-6a

MS (ESI⁺): (M+H)⁺250; HPLC: RT=1.17 min, Method: Z018_S04

### Step 2:

Int-6a (1.55 g, 6.22 mmol) is mixed with Raney-nickel (200 mg) in 30 mL THF and hydrogenated at ambient temperature for 17 h at 50 psi hydrogen pressure. Then the mixture is filtered and concentrated i. vac. Yield: 1.32 g (6.02 mmol; 97%) Int-6b MS (ESI⁺): (M+H)⁺220; HPLC: RT=0.94 min, Method: Z011_S03

### Step 3:

N-2-Boc-amino-3-cyclopropyl-propionic acid (20 g, 87 mmol) are mixed with HCl in dioxane (4N, 150 mL, 600 mmol) at 0° C. and the mixture stirred for 15 min at 0° C. and for 3d at ambient temperature. The mixture is concentrated i. vac., the residue taken up in dioxane and again concentrated i. vac. Yield: 14.4 g (87 mmol; quant.) Int-6c MS (ESI⁺): (M+H)⁺130

### Step 4:

To Int-6c (14.4 g, 87 mmol) in 150 mL MeOH is added thionylchloride (8.0 mL, 110 mmol) under stirring at ambient temperature, and the mixture stirred for 5 h. The mixture is concentrated i. vac., the residue taken up with dioxane, concentrated i. vac., the residue taken up with ACN and again concentrated i. vac. Yield: 15.6 g (87 mmol; quant.) Int-6d

MS (ESI⁺): (M+H)⁺144

### Step 5:

A mixture of Int-6d (7.00 g, 39.0 mmol) and 5-methyl-pyrazine-2-carboxylic acid (7.00 g, 50.7 mmol) in 400 mL THF is stirred at 0° C., TEA (15 mL, 108 mmol) and afterwards CIP (11.5 g, 41.3 mmol) are added and the mixture stirred for 20 min at 0° C. Water is added and the mixture concentrated i. vac. The residue is taken up with water and DCM, the aq. phase is extracted with DCM, the organic layers combined, dried over MgSO₄ and concentrated i. vac.

Yield: 23 g (content: 45%; 39 mmol; quant.) Int-6e

MS (ESI⁺): (M+H)⁺264; HPLC: RT=0.91 min, Method: Z018_S04

### Step 6:

Int-6e (23 g, content: 45%, 39 mmol) in 150 mL MeOH is mixed with 1N NaOH (aq.) (40 mL, 40 mmol) and stirred at ambient temperature for 1 h. Then, 4N NaOH (10 mL, 40 mmol) is added and the mixture stirred at ambient temperature for 1.5 h. The mixture is concentrated i. vac. and the residue taken up with water, acidified with 4n HCl (aq.) to pH 1 and the aq. phase extracted with DCM. The combined organic layers are dried over MgSO₄, concentrated i. vac. and the residue purified by preparative HPLC (C-18 Sunfire at 50° C., eluent gradient (water+0.15% TFA):ACN 83:17->63:37). The product containing fractions are combined, concentrated i. vac., the aq. phase extracted with DCM, the combined organic layers dried over MgSO₄ and concentrated i. vac.

Yield: 7.07 g (28.4 mmol, 72%) Int-6f

MS (ESI⁺): (M+H)⁺250; HPLC: RT=0.80 min, Method: Z018_S04

### Step 7:

Int-6b (100 mg, 0.46 mmol), Int-6f (100 mg, 0.40 mmol) and NMM (265 μL, 2.41 mmol) in 5 mL DCM are stirred at 0° C. and PPA (50% in EtOAc; 470 μL, 0.80 mmol) is added. After stirring for 1 h at 0° C., cooling is removed and the mixture stirred at ambient temperature for 16 h. Water and 5 mL AcOH are added, and the mixture stirred at 90° C. for 45 min, then at 100° C. for 3 h, then at ambient temperature for 3d, and then at 100° C. for 2 h. The mixture is diluted with MeOH, filtrated and the filtrate is purified by HPLC (C-18 Sunfire at 50° C., eluent gradient (water+0.15% TFA):ACN 45:55->25:75). The product containing fractions are combined and freeze dried. The residue is taken up in MeOH, sent over an ion exchange cartridge (Agilent PL-HCO3 MP SPE) and concentrated i. vac. The residue is purified by chiral SFC.

Yield: 54 mg (0.12 mmol; 59%) example 6

Example 6: N-[(1S)-2-cyclopropyl-1-[3-(propan-2-yl)-6-(trifluoromethyl)-3H-
imidazo[4,5-b]pyridin-2-yl]ethyl]-5-methylpyrazine-2-carboxamide

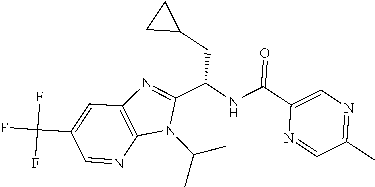 [SMILES: Cc1cnc(C(=O)N[C@@H](CC2CC2)c2nc3cc(C(F)(F)F)cnc3n2C(C)C)cn1]

| | |
|---|---|
| HPLC-MS; Method: Z018_S04; Rₜ [min]: 1.17 | MS: 433 (M + H)⁺ |
| Chiral SFC Rt Method: I_IG_15_MEOH_NH3_001 | Rt [min]: 2.57 |

¹H NMR (400 MHz, DMSO-d₆) δ ppm 0.01-0.08 (m, 1 H) 0.10-0.16 (m, 1 H) 0.32-0.41 (m,
2 H) 0.72-0.82 (m, 1 H) 1.60 (d, J = 6.72 Hz, 3 H) 1.73 (d, J = 6.72 Hz, 3 H)
1.97-2.16 (m, 2 H) 2.60 (s, 3 H) 5.03 (quin, J = 6.78 Hz, 1 H) 5.64 (q, J = 7.48 Hz, 1 H)
8.47 (d, J = 1.77 Hz, 1 H) 8.64 (d, J = 1.01 Hz, 1 H) 8.71 (d, J = 1.39 Hz, 1 H) 9.06 (d,
J = 1.39 Hz, 1 H) 9.26 (d, J = 8.24 Hz, 1 H)

In analogy to example 6, the following products are obtained:

Example 25: N-[(R)-[6-chloro-3-(propan-2-yl)-3H-imidazo[4,5-b]pyridin-2-yl](1-
hydroxycyclobutyl)methyl]-5-methylpyrazine-2-carboxamide

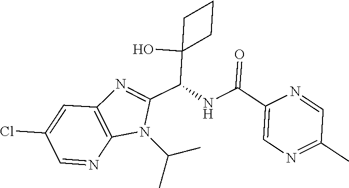 [SMILES: Cc1cnc(C(=O)N[C@H](c2nc3cc(Cl)cnc3n2C(C)C)C2(O)CCC2)cn1]

| | |
|---|---|
| HPLC-MS; Method: Z018_S04; Rₜ [min]: 1.11 | MS: 415 (M + H)⁺ |
| Chiral SFC Rt Method: I_SC_20_MEOH_NH3_001 | Rt [min]: 1.68 |

¹H NMR (400 MHz, DMSO-d₆) δ ppm 1.53-1.73 (m, 7 H) 1.73-1.87 (m, 1 H) 1.92-
2.10 (m, 2 H) 2.20-2.30 (m, 1 H) 2.31-2.41 (m, 1 H) 2.60 (s, 3 H) 5.09 (spt, J = 6.78
Hz, 1 H) 5.62 (d, J = 8.87 Hz, 1 H) 5.83 (s, 1 H) 8.23 (d, J = 2.28 Hz, 1 H) 8.36 (d, J = 2.15
Hz, 1 H) 8.67 (d, J = 1.01 Hz, 1 H) 8.86 (d, J = 8.74 Hz, 1 H) 9.08 (d, J = 1.27 Hz, 1 H)

Example 32: N-[(S)-cyclopropyl[6-fluoro-3-(propan-2-yl)-3H-imidazo[4,5-b]pyridin-2-
yl]methyl]-5-(difluoromethyl)pyrazine-2-carboxamide

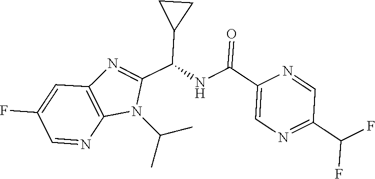 [SMILES: CC(C)n1c([C@@H](NC(=O)c2cnc(C(F)F)cn2)C2CC2)nc2cc(F)cnc21]

| | |
|---|---|
| HPLC-MS; Method: Z011_S03; Rₜ [min]: 0.98 | MS: 405 (M + H)⁺ |

¹H NMR (400 MHz, DMSO-d₆) δ ppm 0.45-0.65 (m, 4 H) 1.54-1.59 (m, 3 H) 1.66-
1.77 (m, 4 H) 2.56-2.63 (m, 1 H) 4.87-5.05 (m, 2 H) 7.21 (m, 1 H) 8.02 (dd, J = 9.44,
2.60 Hz, 1 H) 8.35 (d, J = 3.97 Hz, 1 H) 9.06 (s, 1 H) 9.29 (s, 1 H) 9.51 (br d, J = 7.86 Hz,
1 H)

Example 44: N-[(S)-cyclopropyl[6-fluoro-3-(propan-2-yl)-3H-imidazo[4,5-b]pyridin-2-
yl]methyl]-5-methylpyrazine-2-carboxamide

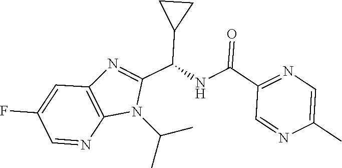 [SMILES: Cc1cnc(C(=O)N[C@H](c2nc3cc(F)cnc3n2C(C)C)C2CC2)cn1]

| | |
|---|---|
| HPLC-MS; Method: Z018_S04; Rₜ [min]: 1.21 | MS: 459 (M + H)⁺ |
| Chiral SFC Rt Method: Method I_SC_20_MEOH_NH3_001 | Rt [min]: 2.83 |

¹H NMR (400 MHz, DMSO-d₆) δ ppm 0.43-0.65 (m, 4 H) 1.50-1.60 (m, 3 H) 1.65-1.78 (m,
4 H) 2.60 (s, 3 H) 4.85-5.05 (m, 2 H) 8.02 (dd, J = 9.50, 2.66 Hz, 1 H) 8.35
(dd, J = 2.60, 1.84 Hz, 1 H) 8.64 (d, J = 0.89 Hz, 1 H) 9.05 (d, J = 1.27 Hz, 1 H) 9.26 (d,
J = 8.11 Hz, 1 H)

### Example 8

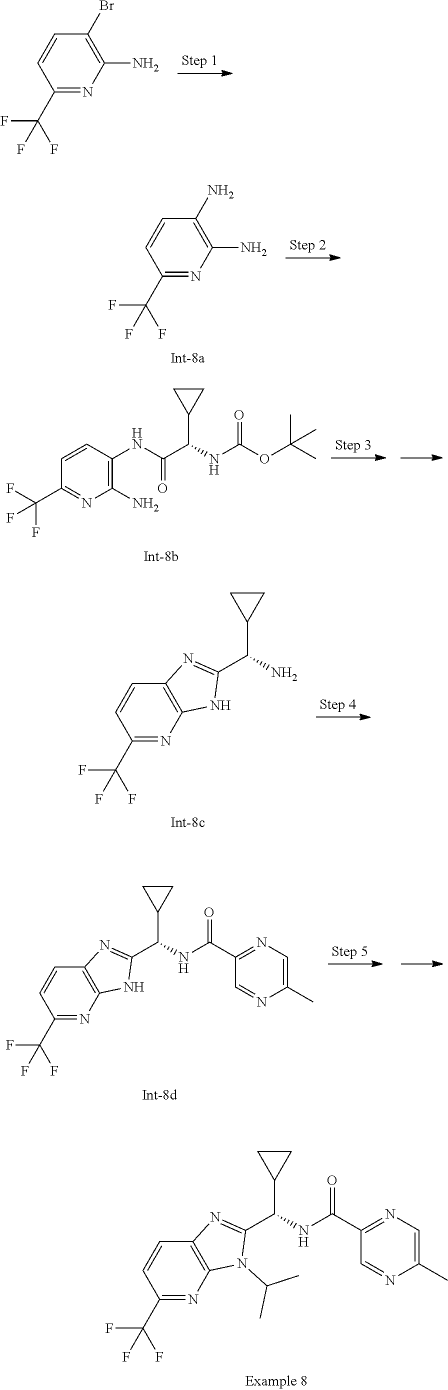 [SMILES: Nc1nc(C(F)(F)F)ccc1Br ; Nc1ccc(C(F)(F)F)nc1N ; CC(C)(C)OC(=O)N[C@H](C(=O)Nc1ccc(C(F)(F)F)nc1N)C1CC1 ; N[C@H](c1nc2ccc(C(F)(F)F)nc2[nH]1)C1CC1 ; Cc1cnc(C(=O)N[C@H](c2nc3ccc(C(F)(F)F)nc3[nH]2)C2CC2)cn1 ; Cc1cnc(C(=O)N[C@H](c2nc3ccc(C(F)(F)F)nc3n2C(C)C)C2CC2)cn1]

### Step 1:

To a mixture of 3-bromo-6-trifluoromethyl-pyridine-2-amine (2.00 g, 8.30 mmol) and NH₃ (aq.) (30%; 5.45 mL, 41.5 mmol) in 12 mL DMF are added acetylacetone (342 μL, 3.32 mmol) and cupric acetylacetonate (217 mg, 0.83 mmol) under argon and the mixture stirred at 90° C. for 18 h. 100 mL EtOAc and 60 mL water are added and the aq. phase extracted with EtOAc. The combined organic layers are dried over Na₂SO₄ and concentrated i. vac. The residue is purified by chromatography on a silica column (eluent: DCM:MeOH 98:2), the product containing fractions are combined and concentrated i. vac. Yield: 1.51 g (content: 98%; 8.30 mol; quant.) Int-8a. MS (ESI⁺): (M+H)⁺178; HPLC: RT=0.69 min, Method: Z011_S03

### Step 2:

To Int-8a (453 mg, 2.56 mmol) and (2S)-2-Boc-amino-2-cyclopropyl-acetic acid (500 mg, 2.33 mmol) in 10 mL DCM is added NMM (894 μL, 8.05 mmol) and PPA (50 wt % in AcOH, 2.7 mL, 4.41 mmol) at 0° C. and then stirred at 0° C. for 4.5 h. DCM and NaHCO₃ (aq., 5%) are added and the mixture stirred vigorously. The organic layer is dried over Na₂SO₄, concentrated i. vac., the residue taken up in diethylether and concentrated i. vac. Yield: 1.00 g (content: 96%; 2.56 mmol; quant.) Int-8b. MS (ESI⁺): (M+H)⁺375; HPLC: RT=0.97 min, Method: Z011_S03

### Step 3:

A mixture of Int-8b (900 mg, 2.41 mmol), ZnBr₂ (1.08 g, 4.81 mmol) and n-butyl acetate is stirred at 110° C. for 2.5d. EtOAc and NaHCO₃ (aq., 5%) are added and the mixture stirred vigorously.

The mixture is filtered, the solid dried at ambient temperature and used without further purification.

Yield: 1.20 g (content: 50%; 2.34 mmol; 97%) Int-8c

MS (ESI⁺): (M+H)⁺257; HPLC: RT=0.64 min, Method: Z011_S03

### Step 4:

Int-8c (50%; 1.74 g, 3.40 mmol) and 2-methyl-pyrazine-5-carboxylic acid (100 mg, 0.40 mmol) in 30 mL DCM are stirred at 0° C. and NMM (1.50 mL, 14.4 mmol) and PPA (50% in EtOAc; 4.0 mL, 3.40 mmol) are added. After stirring for 1 h at 0° C., cooling reduced to reach ambient temperature over 16 h. EtOAc and NaHCO₃ (aq., 5%) are added and the mixture stirred vigorously. The mixture is filtered and the solid dried at ambient temperature.

Yield: 1.36 g (content: 94%; 3.40 mmol; quant.) Int-8d

MS (ESI⁺): (M+H)⁺377; HPLC: RT=0.69 min, Method: Z011_S03

### Step 5:

To Int-8d (600 mg, 1.60 mmol) in 7.0 mL DMF is added Cs₂CO₃ (779 mg, 2.39 mmol) and isopropyl methanesulfonate (100 μL, 3.19 mmol) and the mixture stirred at 90° C. for 17 h. Then more isopropyl methanesulfonate (385 μL, 0.83 mmol) is added and the mixture stirred at 90° C. for 8 h. Afterwards, EtOAc is added and the mixture filtrated. The filtrate is concentrated i. vac, the residue taken up in MeOH, filtered and the filtrate purified via prep. HPLC (C-18 X-Bridge at 60° C., eluent (water+0.15% NH₃):ACN mixture). The product containing fractions are combined and freeze-dried.

Yield: 107 mg (0.26 mmol; 16%) example 8

Example 8: N-[(S)-cyclopropyl[3-(propan-2-yl)-5-(trifluoromethyl)-3H-imidazo[4,5-
b]pyridin-2-yl]methyl]-5-methylpyrazine-2-carboxamide

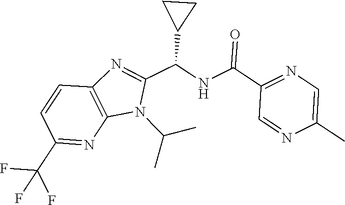 [SMILES: Cc1cnc(C(=O)N[C@H](c2nc3ccc(C(F)(F)F)nc3n2C(C)C)C2CC2)cn1]

| | |
|---|---|
| HPLC-MS; Method: Z011_S03; Rₜ [min]: 1.08 | MS: 419 (M + H)⁺ |
| Chiral SFC Rt Method: I_SB_10_IPA_NH3_001 | Rt [min]: 1.80 |

¹H NMR (600 MHz, DMSO-d₆) δ ppm 0.48-0.63 (m, 4 H) 1.57 (d, J = 6.71 Hz, 3 H)
1.70-1.75 (m, 4 H) 2.60 (s, 3 H) 4.96 (d, J = 6.75 Hz, 1 H) 5.03 (dd, J = 8.77, 8.03 Hz, 1
H) 7.73 (d, J = 8.29 Hz, 1 H) 8.29 (d, J = 8.33 Hz, 1 H) 8.64 (s, 1 H) 9.34 (d, J = 7.96 Hz, 1
H)

### Example 11

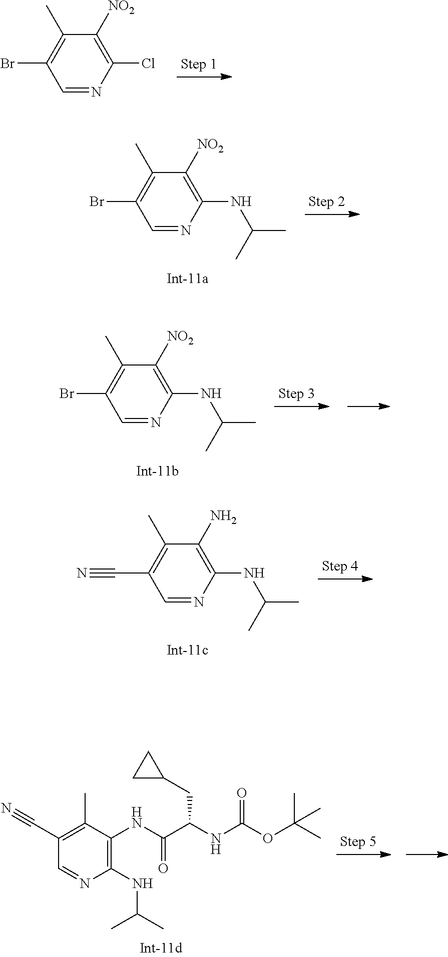 [SMILES: Cc1c(Br)cnc(Cl)c1[N+](=O)[O-] ; Cc1c(Br)cnc(NC(C)C)c1[N+](=O)[O-] ; Cc1c(Br)cnc(NC(C)C)c1[N+](=O)[O-] ; Cc1c(C#N)cnc(NC(C)C)c1N ; Cc1c(C#N)cnc(NC(C)C)c1NC(=O)[C@H](CC1CC1)NC(=O)OC(C)(C)C]

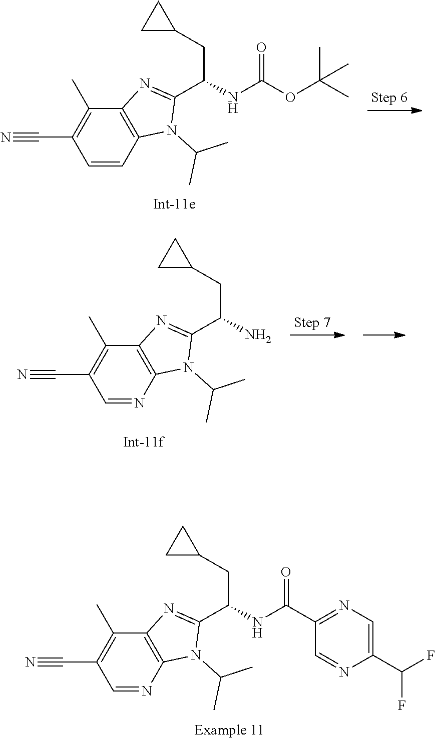 [SMILES: Cc1c(C#N)ccc2c1nc([C@H](CC1CC1)NC(=O)OC(C)(C)C)n2C(C)C ; Cc1c(C#N)cnc2c1nc([C@@H](N)CC1CC1)n2C(C)C ; Cc1c(C#N)cnc2c1nc([C@H](CC1CC1)NC(=O)c1cnc(C(F)F)cn1)n2C(C)C]

### Step 1:

To isopropylamine (60 mL, 704 mmol) is added 5-bromo-2-chloro-4-methyl-3-nitropyridine (20.0 g, 79.5 mmol) over 2 min under stirring and the mixture stirred at ambient temperature for 17 h. Afterwards, the mixture is concentrated i. vac., the residue treated with water, filtrated, the solid washed with water, taken up in ACN and dried i. vac. at 45° C. Yield: 21.2 g (77.3 mmol; 97%) Int-11a. MS (ESI⁺): (M+H)⁺274/276 (Br); HPLC: RT=1.22 min, Method: Z018_S04

### Step 2:

Int-11a (10.0 g, 36.5 mmol) is mixed with Raney-nickel (200 mg) in 150 mL THF and hydrogenated at ambient temperature for 18 h at 50 psi hydrogen pressure. Then the mixture is filtered and concentrated and dried i. vac. Yield: 9.00 g (content: 98%; 36.1 mmol; 99%) Int-11b MS (ESI⁺): (M+H)⁺244/246 (Br); HPLC: RT=0.94 min, Method: Z011_S03

### Step 3:

To a mixture of Int-11b (27.6 g, 111 mmol) with Zn(CN)₂ (13.3 g, 113 mmol) in 100 mL NMP under argon is added tetrakis(tripenylphosphine)-palladium(0) (6.40 g, 5.54 mmol) and the mixture stirred at 115° C. for 45 min. Then 300 mL DCM and 300 mL water are added, the mixture stirred vigorously and filtered. The organic layer is washed with water, dried over MgSO₄ and concentrated i. vac. The residue is taken up in DCM, mixed with extrelut, concentrated i. vac. and purified via column chromatography on silica gel (eluent-gradient: PE:EtOAc 85:15->55:45). The aq. phase from quenching is extracted with EtOAc, the combined organic layers washed with NaCl (aq.) and dried over MgSO₄ and concentrated i. vac. The residue is purified by column chromatography on silica gel (eluent-gradient: PE:EtOAc 85:15->55:45). The product containing fractions are each combined and concentrated i. vac., the residues are combined. Yield: 13.6 g (71.5 mmol; 65%) Int-11c

MS (ESI⁺): (M+H)⁺191; HPLC: RT=0.81 min, Method: Z011_S03

### Step 4:

A mixture of Int-11c (13.6 g, 71.5 mmol), (S)-2-(Boc-amino)-3-cyclopropyl-propanoic acid and NMM (38.5 mL, 350 mmol) in 1.0 L DCM is cooled to −10° C. and under stirring PPA (50% in EtOAc; 84.6 mL, 144 mmol) is added within 8 min. Under stirring, cooling is reduced to reach ambient temperature within 16 h. 200 mL NaHCO₃ (aq., 5%) is added and the organic phase washed with water. 250 mL water is added and the mixture acidified to pH 4 with KHSO₄ (aq. 0.5N) and stirred vigorously. The organic layer is washed with water, dried over MgSO₄ and concentrated i. vac. The residue is treated with di-isopropyl-ether and dried i. vac. at 45° C.

Yield: 20.6 g (51.4 mmol; 73%) Int-11d

MS (ESI⁺): (M+H)⁺402; HPLC: RT=1.06 min, Method: Z011_S03

### Step 5:

A mixture of Int-11d (20.2 g, 50.2 mmol) and K₂CO₃ (8.33 g, 60.3 mmol) in 300 mL 2-propanol is stirred at 85° C. for 22 h and at 90° C. for 34 h. 200 mL DCM are added, filtered and the filtrate is concentrated i. vac. The residue is taken up with DCM/MeOH, silica gel added and the mixture concentrated i. vac. The residue is used for purification via column chromatography on silica gel (eluent DCM:EtOH 98:2). The product containing fractions are combined and concentrated i. vac. Yield: 16.0 g (41.7 mmol; 83%) Int-11e

MS (ESI⁺): (M+H)⁺384; HPLC: RT=1.16 min, Method: Z011_S03

### Step 6:

To Int-11e (16.0 g, 41.7 mmol) in 250 mL DCM is slowly added solution of HCl in dioxane (4N; 52.2 mL, 209 mmol). The mixture is stirred at ambient temperature for 16 h and concentrated i. vac.

Yield: 14.9 mg (41.7 mmol; quant.) Int-11f

MS (ESI⁺): (M+H)⁺284; HPLC: RT=0.95 min, Method: Z011_S03

### Step 7:

A mixture from Int-11f (300 mg, 0.84 mmol), 5-difluoromethyl-pyrazine-2-carboxylic acid (188 mg, 1.08 mmol) and pyridine (2.1 mL, 26 mmol) is stirred at 0° C., PPA (50% in EtOAc; 0.7 mL, 1.2 mmol) is added and the mixtures stirred for 30 min at 0° C. and 16 h at ambient temperature. THF and water are added and the mixture is purified by prep. HPLC (C-18 X-Bridge 10 μm, eluent gradient (water +0.1% NH₃):ACN 52:48->42:58). The product containing fractions are combined, concentrated i. vac., the residue taken up in ACN and water and freeze-dried.

Yield: 125 mg (284 μmol; 42%) example 11

Example 11: N-[(1S)-1-[6-cyano-7-methyl-3-(propan-2-yl)-3H-imidazo[4,5-b]pyridin-
2-yl]-2-cyclopropylethyl]-5-(difluoromethyl)pyrazine-2-carboxamide

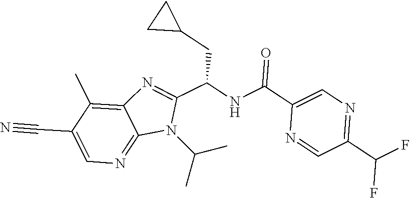 [SMILES: Cc1c(C#N)cnc2c1nc([C@H](CC1CC1)NC(=O)c1cnc(C(F)F)cn1)n2C(C)C]

| | |
|---|---|
| HPLC-MS; Method: Z011_S03; Rₜ [min]: 1.09 | MS: 440 (M + H)⁺ |
| Chiral SFC Rt Method: I_SC_20_IPA_NH3_001 | Rt [min]: 1.70 |

¹H NMR (400 MHz, DMSO-d₆) δ ppm 0.02-0.25 (m, 2 H) 0.30-0.42 (m, 2 H) 0.73-
0.84 (m, 1 H) 1.57-1.62 (m, 3 H) 1.65-1.77 (m, 3 H) 2.00-2.18 (m, 2 H) 2.72 (s, 3
H) 5.02 (dt, J = 13.50, 6.69 Hz, 1 H) 5.63 (q, J = 7.65 Hz, 1 H) 7.20 (m, 1 H) 8.66 (s, 1 H)
9.05 (s, 1 H) 9.28 (s, 1 H) 9.52 (br d, J = 7.98 Hz, 1 H)

In analogy to example 11, the following product is obtained:

Example 14: N-[(1S)-1-[6-cyano-7-methyl-3-(propan-2-yl)-3H-imidazo[4,5-b]pyridin-
2-yl]-2-cyclopropylethyl]-5-methylpyrazine-2-carboxamide

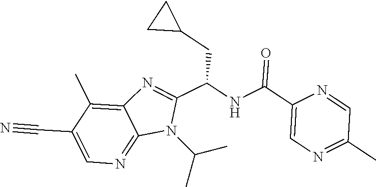 [SMILES: Cc1cnc(C(=O)N[C@@H](CC2CC2)c2nc3c(C)c(C#N)cnc3n2C(C)C)cn1]

| | |
|---|---|
| HPLC-MS; Method: Z018_S04; Rₜ [min]: 1.11 | MS: 404 (M + H)⁺ |
| Chiral SFC Rt Method: I_SC_20_IPA_NH3_001 | Rt [min]: 3.54 |

¹H NMR (400 MHz, DMSO-d₆) δ ppm 0.02-0.11 (m, 1 H) 0.12-0.21 (m, 1 H) 0.30-0.42 (m,
2 H) 0.71-0.82 (m, 1 H) 1.53-1.60 (m, 3 H) 1.66-1.73 (m, 3 H) 1.66-1.73
(m, 2 H) 1.85-2.25 (m, 2 H) 2.59 (s, 3 H) 2.71 (s, 3 H) 5.01 (spt, J = 6.78 Hz, 1 H) 5.57-
5.65 (m, 1 H) 8.63 (d, J = 1.01 Hz, 1 H) 8.66 (s, 1 H) 9.05 (d, J = 1.39 Hz, 1 H) 9.27 (d,
J = 8.11 Hz, 1 H)

### Example 16

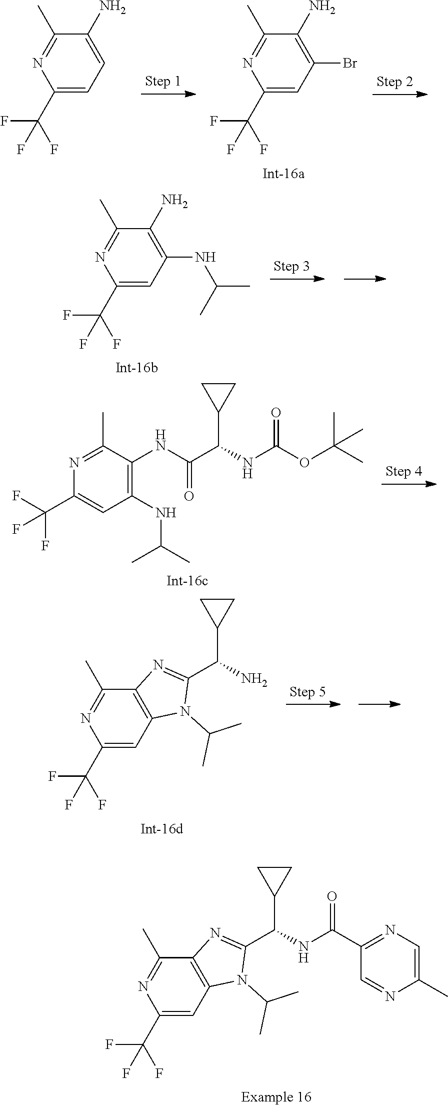

### Step 1:

To 2-methyl-6-trifluoromethyl-pyridine-3-amine (20 g, 108 mmol) in 230 mL ACN is portionwise added NBS (20.2 g, 113 mmol) within 3 min under stirring at ambient temperature. Stirring is continued for 2.5 h. Then the mixture is concentrated i. vac., the residue taken um in DCM and washed with water. The organic phase is dried over MgSO₄ and concentrated i. vac. Yield: 27.2 g (107 mmol; 99%) Int-16a. MS (ESI⁺): (M+H)⁺ 255; HPLC: RT=0.99 min, Method: Z018_S04

### Step 2:

In an autoclave, Int-16a (27.0 g, 106 mmol) is mixed with isopropylamine (91.4 mL, 1.06 mol), 169 mL water, CuI (1.21 g, 6.35 mmol) and 1-pyridine-2-yl-ethanone oxime (1.19 g, 8.47 mmol). The mixture is stirred for 24 h at 90° C. Then THF and water are added and the mixture concentrated i. vac. The residue is extracted with EtOAc, the combined organic layers dried over MgSO₄ and concentrated i. vac. The residue is mixed with DCM, THF and extrelut, the mixture concentrated i. vac. and the residue purified via column chromatography on silica gel (eluent gradient: PE:EtOAc 67:33->37:63). The product containing fractions are combined and concentrated i. vac. Yield: 10.1 g (43.3 mol; 41%) Int-16b

MS (ESI⁺): (M+H)⁺234; HPLC: RT=0.92 min, Method: Z011_S03

### Step 3:

A mixture of Int-16b (10.1 g, 42 mmol) and (2S)-2-Boc-amino-2-cyclopropyl-acetic acid (9.95 g, 46 mmol) in 100 mL pyridine is cooled to −10° C. Under stirring, PPA (50% in EtOAc, 37.1 mL, 63 mmol) is added over 8 min and the mixture stirred for 1 h at −5° C. The mixture is poured into water at 0° C., vigorously stirred, adjusted to pH 8 by addition of NH₃ (aq., conc.) and vigorously stirred again. The formed solid is filtered, washed with water and dried at 60° C. i. vac.

Yield: 17.0 g (39.6 mmol; 94%) Int-16c. MS (ESI⁺): (M+H)⁺431; HPLC: RT=1.06 min, Method: Z011_S03

### Step 4:

Int-16c (17.0 g, 37.5 mmol) and ZnBr₂ (18.6 g, 82.5 mmol) in n-butyl acetate are stirred at 115° C. for 21 h and at 135° C. for 2 h. The mixture is poured into ice water and stirred. The pH is adjusted to 8.5 by addition of NH₃ (aq., conc.), celite added, stirred and filtrated. The solid is washed with EtOAc. The organic layer is washed with NaCl (aq.), dried over MgSO₄ and concentrated i. vac. The residue is taken up with EtOAc and purified by column chromatography on silica gel (eluent gradient: EtOAc:(EtOH+5% NH₃ (aq., conc.)) 97:3->80:20). Product containing fractions are combined and concentrated i. vac. Yield: 11.5 g (36.9 mmol; 98%) Int-16d.

MS (ESI⁺): (M+H)⁺313; HPLC: RT=0.93 min, Method: Z011_S03

### Step 5:

Under nitrogen, Int-16d (60.0 g, 192 mmol) is added to 5-methyl-pyrazine-2-carboxylic acid (188 mg, 1.08 mmol) with TEA (80 mL, 576 mmol) in 500 mL EtOAc and the mixture cooled to −5° C. PPA (50% in EtOAc; 149 mL, 250 mmol) is added under stirring and cooling to keep the temperature below 0° C. Then cooling is removed and the mixture stirred for 45 min at ambient temperature. 1.0 L water is added and the mixture is stirred for 5 min. The organic phase is washed twice with 500 mL NaCl solution (aq., halfconc.) with 5 mL NH₃ (aq., conc.). 10 g charcoal is added and stirred for 10 min, then filtrated, dried over MgSO₄ and concentrated i. vac. The residue is taken up with DCM and diethylether and each concentrated i. vac. again, then dried i. vac. at ambient temperature. The residue is taken up with 80 mL di-isopropyl-ether and then portionwise a total of 320 mL n-heptane is added with vigorous stirring in between. The solid is filtered, washed with 200 mL n-heptane and dried i. vac. at 55° C. Then the solid is taken into 1.46 L water and stirred for 22 h at ambient temperature, filtered off, washed with 1.5 L water and dried for 22 h at 65° C. under nitrogen.

Yield: 69.6 g (162 mmol; 84%) example 16

Example 16: N-[(S)-cyclopropyl[4-methyl-1-(propan-2-yl)-6-(trifluoromethyl)-1H-
imidazo[4,5-c]pyridin-2-yl]methyl]-5-methylpyrazine-2-carboxamide

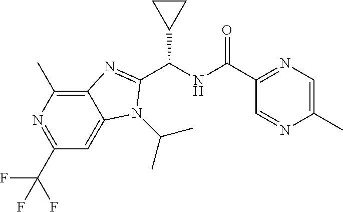 [SMILES: Cc1cnc(C(=O)N[C@H](c2nc3c(C)nc(C(F)(F)F)cc3n2C(C)C)C2CC2)cn1]

| | |
|---|---|
| HPLC-MS; Method: Z011_S03; Rₜ [min]: 1.04 | MS: 433 (M + H)⁺ |
| Chiral SFC Rt Method: I_SC_10_MEOH_NH3_001 | Rt [min]: 2.65 |

¹H NMR (400 MHz, DMSO-d₆) δ ppm 0.44-0.68 (m, 4 H) 1.45-1.52 (m, 3 H) 1.57-
1.64 (m, 3 H) 1.72-1.84 (m, 1 H) 2.60 (s, 3 H) 2.77 (s, 3 H) 4.95-5.07 (m, 2 H) 8.04
(s, 1 H) 8.65 (d, J = 1.01 Hz, 1 H) 9.04 (d, J = 1.27 Hz, 1 H) 9.35 (d, J = 7.86 Hz, 1 H)

In analogy to example 16, the following products are obtained:

Example 22: N-[(S)-cyclopropyl[4-methyl-1-(propan-2-yl)-6-(trifluoromethyl)-1H-
imidazo[4,5-c]pyridin-2-yl]methyl]-5-(difluoromethyl)pyrazine-2-carboxamide

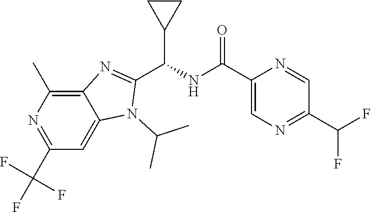 [SMILES: Cc1nc(C(F)(F)F)cc2c1nc([C@@H](NC(=O)c1cnc(C(F)F)cn1)C1CC1)n2C(C)C]

| | |
|---|---|
| | MS: 469 (M + H)⁺ |
| Chiral SFC Rt Method: I_SC_05_IPA_NH3_001 | Rt [min]: 3.08 |

¹H NMR (400 MHz, DMSO-d₆) δ ppm 0.46-0.66 (m, 4 H) 1.48-1.55 (m, 3 H) 1.58-
1.64 (m, 3 H) 1.75-1.85 (m, 1 H) 2.77 (s, 3 H) 4.96-5.07 (m, 2 H) 7.21 (m, 1 H) 8.04
(s, 1 H) 9.06 (s, 1 H) 9.27 (s, 1 H) 9.61 (d, J = 7.73 Hz, 1 H)

Example 29: N-[(S)-cyclopropyl[1-(2,2-difluoroethyl)-4-methyl-6-(trifluoromethyl)-1H-
imidazo[4,5-c]pyridin-2-yl]methyl]-5-(difluoromethyl)pyrazine-2-carboxamide

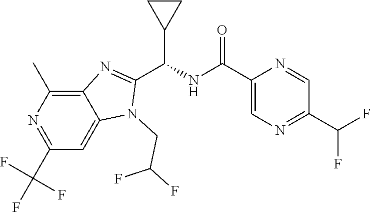 [SMILES: Cc1nc(C(F)(F)F)cc2c1nc([C@@H](NC(=O)c1cnc(C(F)F)cn1)C1CC1)n2CC(F)F]

| | |
|---|---|
| HPLC-MS; Method: Z011_S03; Rₜ [min]: 1.04 | MS: 491 (M + H)⁺ |
| Chiral SFC Rt Method: I_SA_10_MEOH_NH3_001 | Rt [min]: 1.25 |

¹H NMR (400 MHz, DMSO-d₆) δ ppm 0.43-0.53 (m, 2 H) 0.57-0.68 (m, 2 H) 1.91-
2.00 (m, 1 H) 2.79 (s, 3 H) 4.87-4.96 (m, 1 H) 4.98-5.21 (m, 2 H) 6.50 (m, 1 H) 7.20
(m, 1 H) 8.13 (s, 1 H) 9.04 (s, 1 H) 9.24-9.27 (m, 1 H) 9.63 (d, J = 7.86 Hz, 1 H)

Example 35: N-[(S)-cyclopropyl[3-(2,2-difluoroethyl)-6-(trifluoromethyl)-3H-
imidazo[4,5-c]pyridin-2-yl]methyl]-5-(difluoromethyl)pyrazine-2-carboxamide

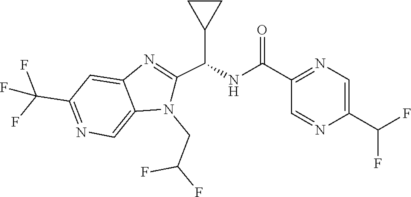 [SMILES: O=C(N[C@H](c1nc2cc(C(F)(F)F)ncc2n1CC(F)F)C1CC1)c1cnc(C(F)F)cn1]

| | |
|---|---|
| HPLC-MS; Method: Z011_S03; Rₜ [min]: 1.00 | MS: 477 (M + H)⁺ |
| Chiral SFC Rt Method: I_IG_15_IPA_NH3_001 | Rt [min]: 1.87 |

¹H NMR (400 MHz, DMSO-d₆) δ ppm 0.43-0.71 (m, 4 H) 1.84-1.99 (m, 1 H) 4.90-
5.02 (m, 1 H) 5.02-5.26 (m, 2 H) 6.55 (m, 1 H) 7.21 (m, 1 H) 8.24 (2, 1 H) 9.05 (s, 1
H) 9.15 (s, 1 H) 9.25-9.28 (m, 1 H) 9.65 (br s, 1 H)

Example 39: N-[(S)-cyclopropyl[1-(2,2-difluoroethyl)-4-methyl-6-(trifluoromethyl)-1H-
imidazo[4,5-c]pyridin-2-yl]methyl]-5-methylpyrazine-2-carboxamide

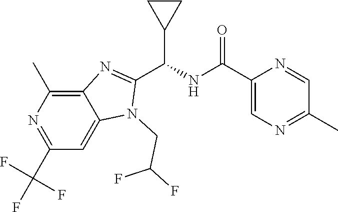 [SMILES: Cc1cnc(C(=O)N[C@H](c2nc3c(C)nc(C(F)(F)F)cc3n2CC(F)F)C2CC2)cn1]

| | |
|---|---|
| HPLC-MS; Method: Z011_S03; Rₜ [min]: 1.00 | MS: 455 (M + H)⁺ |
| Chiral SFC Rt Method: I_IG_15_IPA_NH3_001 | Rt [min]: 1.89 |

¹H NMR (400 MHz, DMSO-d₆) δ ppm 0.40-0.53 (m, 2 H) 0.53-0.68 (m, 2 H) 1.88-
1.97 (m, 1 H) 2.59 (s, 3 H) 2.79 (s, 3 H) 4.85-4.95 (m, 1 H) 4.95-5.21 (m, 2 H) 6.49
(m, 1 H) 8.12 (s, 1 H) 8.63 (d, J = 1.01 Hz, 1 H) 9.03 (d, J = 1.39 Hz, 1 H) 9.36 (d, J = 7.86
Hz, 1 H)

Example 46: N-[(S)-cyclopropyl[1-(2,2-difluoroethyl)-6-(trifluoromethyl)-1H-
imidazo[4,5-c]pyridin-2-yl]methyl]-5-(difluoromethyl)pyrazine-2-carboxamide

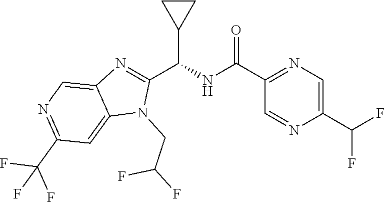 [SMILES: O=C(N[C@H](c1nc2cnc(C(F)(F)F)cc2n1CC(F)F)C1CC1)c1cnc(C(F)F)cn1]

| | |
|---|---|
| HPLC-MS; Method: Z011_S03; Rₜ [min]: 0.98 | MS: 477 (M + H)⁺ |
| Chiral SFC Rt Method: I_SA_10_IPA_NH3_001 | Rt [min]: 3.43 |

¹H NMR (400 MHz, DMSO-d₆) δ ppm 0.43-0.55 (m, 2 H) 0.56-0.70 (m, 2 H) 1.88-
1.97 (m, 4 H) 4.95-5.02 (m, 1 H) 5.03-5.16 (m, 2 H) 6.51 (m, 1 H) 7.21 (s, 1 H) 8.32
(s, 1 H) 9.04 (s, 1 H) 9.13 (s, 1 H) 9.26 (s, 1 H) 9.63 (d, J = 7.98 Hz, 1 H)

### Example 27

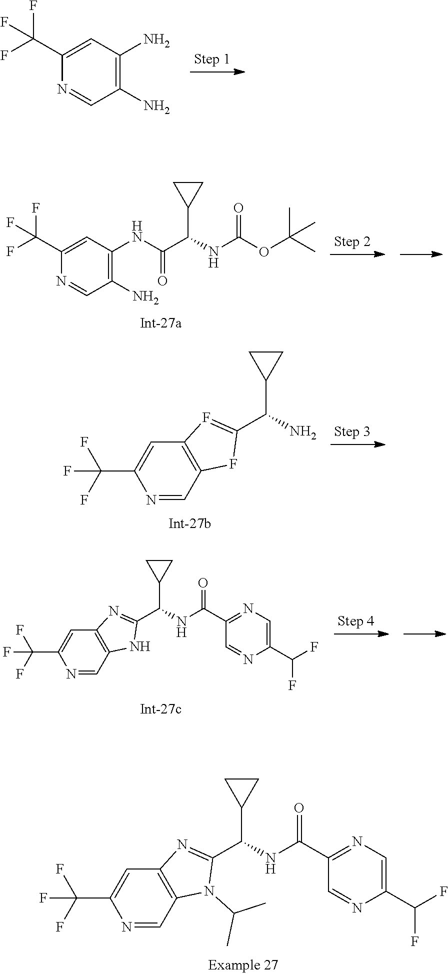

### Step 1:

A mixture of 4,5-di-amino-2-(trifluoromethyl)-pyridine (605 mg, 3.42 mmol), (2S)-2-Boc-amino-2-cyclopropyl-acetic acid (700 mg, 3.26 mmol) and NMM (1.25 mL, 11 mmol) in 40 mL DCM is cooled to −10° C. and PPA (50% in EtOAc, 3.8 mL, 6.60 mmol) is added under stirring. After 1.25 h, icebath is removed, DCM and NaHCO₃ (aq., 5%) added and the mixture vigorously stirred at ambient temperature. The organic layer is dried over NaₛSO₄ and concentrated i. vac. The residue is taken up with diethyl-ether and concentrated i. vac.

Yield: 1.50 g (content: 75%; 3.01 mmol; 92%) Int-27a

MS (ESI⁺): (M+H)⁺375; HPLC: RT=0.88 min, Method: Z011_S03

### Step 2:

A mixture from Int-27a (810 mg, 2.3 mmol) and ZnBr₂ (974 mg, 4.33 mmol) in 15 mL n-butyl acetate is stirred at 110° C. for 19 h. EtOAc and NaHCO₃ (aq., 5%) are added, the mixture vigorously stirred and the aq. layer extracted with EtOAc. The combined organic layers are washed with water, dried over Na₂SO₄ and concentrated i. vac.

Yield: 600 mg (2.3 mmol; quant.) Int-27b

MS (ESI⁺): (M+H)⁺257; HPLC: RT=0.58 min, Method: Z011_S03

### Step 3:

To a mixture of Int-27b (221 mg, 0.86 mmol) with 5-(difluoromethyl)-pyrazine-2-carboxylic acid (150 mg, 0.86 mmol) and NMM (379 μL, 2.72 mmol) in 10 mL DCM is cooled to 0° C. and PPA (50% in EtOAc, 1.0 mL, 1.74 mmol) is added under stirring. Icebath is removed and stirring continued at ambient temperature for 17 h. DCM and NaHCO₃ (aq., 5%) are added and the mixture vigorously stirred at ambient temperature. The organic layer is dried over NaₛSO₄ and concentrated i. vac.

Yield: 304 mg (0.74 mmol; 86%) Int-27c

MS (ESI⁺): (M+H)⁺413; HPLC: RT=0.72 min, Method: Z011_S03

### Step 4:

To a mixture of Int-27c (304 mg, 0.74 mmol), triphenylphosphine (290 mg, 1.11 mmol) and isopropanol (284 μL, 3.69 mmol) in 5 mL THF at 0° C. is added diisopropyl-azodicarboxylate (40% in toluene, 543 μL, 1.11 mmol) and stirred at 0° C. for 3.5 h and at ambient temperature for 17 h. Then, triphenylphosphine (100 mg, 0.38 mmol) and diisopropyl-azodicarboxylate (40% in toluene, 543 μL, 0.41 mmol) are added at 0° C. and stirred for 5.5 h to reach ambient temperature. Water is added, stirred vigorously and extracted with EtOAc. The combined organic layers are dried over Na₂SO₄ and concentrated i. vac. The residue is taken up in DCM and purified via column chromatography on silica gel (eluent DCM:MeOH 98:2). The product containing fractions are combined and concentrated i. vac. The residue is further purified by chiral SFC.

Yield: 68 mg (0.15 mmol, 20%) example 27

Example 27: N-[(S)-cyclopropyl[3-(propan-2-yl)-6-(trifluoromethyl)-3H-imidazo[4,5-
c]pyridin-2-yl]methyl]-5-(difluoromethyl)pyrazine-2-carboxamide

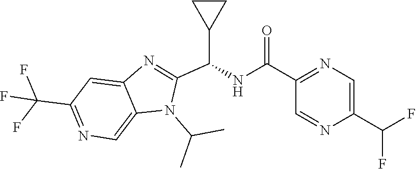 [SMILES: CC(C)n1c([C@@H](NC(=O)c2cnc(C(F)F)cn2)C2CC2)nc2cc(C(F)(F)F)ncc21]

| | |
|---|---|
| HPLC-MS; Method: Z003_S05; Rₜ [min]: 1.14 | MS: 455 (M + H)⁺ |
| Chiral SFC Rt Method: I_IG_15_IPA_NH3_001 | Rt [min]: 1.59 |

¹H NMR (400 MHz, DMSO-d₆) δ ppm 0.49-0.67 (m, 4 H) 1.52-1.60 (m, 3 H) 1.64-
1.70 (m, 3 H) 1.70-1.82 (m, 1 H) 4.99-5.11 (m, 2 H) 7.21 (m, 1 H) 8.19 (s, 1 H) 9.06
(s, 1 H) 9.28 (s, 1 H) 9.33 (s, 1 H) 9.63 (d, J = 7.60 Hz, 1 H)

Example 43: N-[(S)-cyclopropyl[1-(propan-2-yl)-6-(trifluoromethyl)-1H-imidazo[4,5-
c]pyridin-2-yl]methyl]-5-methylpyrazine-2-carboxamide

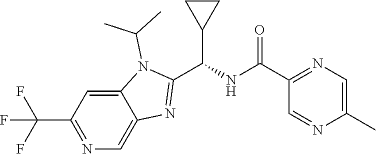 [SMILES: Cc1cnc(C(=O)N[C@H](c2nc3cnc(C(F)(F)F)cc3n2C(C)C)C2CC2)cn1]

| | |
|---|---|
| | MS: 419 (M + H)⁺ |
| Chiral SFC Rt Method: I_IG_25_MEOH_NH3_001 | Rt [min]: 3.20 |

¹H NMR (400 MHz, DMSO-d₆) δ ppm 0.44-0.65 (m, 4 H) 1.50-1.58 (m, 3 H) 1.62-
1.68 (m, 3 H) 1.68-1.79 (m, 1 H) 2.60 (s, 3 H) 4.97-5.09 (m, 2 H) 8.19 (s, 1 H) 8.65
(d, J = 1.01 Hz, 1 H) 9.04 (d, J = 1.27 Hz, 1 H) 9.31 (s, 1 H) 9.38 (d, J = 7.86 Hz, 1 H)

### Example 28

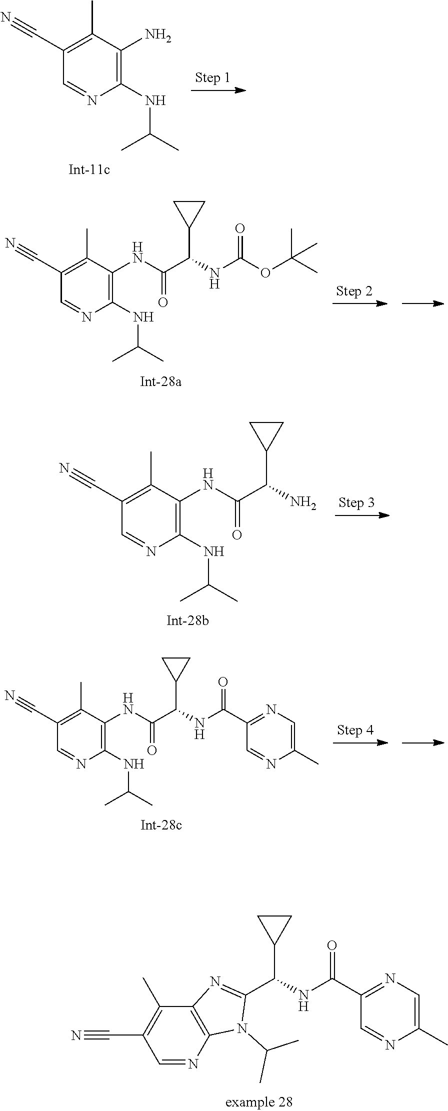 [SMILES: Cc1c(C#N)cnc(NC(C)C)c1N ; Cc1c(C#N)cnc(NC(C)C)c1NC(=O)[C@@H](NC(=O)OC(C)(C)C)C1CC1 ; Cc1c(C#N)cnc(NC(C)C)c1NC(=O)[C@@H](N)C1CC1 ; Cc1cnc(C(=O)N[C@H](C(=O)Nc2c(NC(C)C)ncc(C#N)c2C)C2CC2)cn1 ; Cc1cnc(C(=O)N[C@H](c2nc3c(C)c(C#N)cnc3n2C(C)C)C2CC2)cn1]

### Step 1:

A mixture of Int-11c (365 mg, 1.82 mmol), (2S)-2-Boc-amino-2-cyclopropyl-acetic acid (471 mg, 2.19 mmol) and NMM (1.2 mL, 10.9 mmol) in 25 mL DCM is cooled to −5° C. and PPA (50% in EtOAc, 2.1 mL, 3.65 mmol) is added under stirring. After 10 min, cooling is removed and the mixture stirred at ambient temperature for 3.5d. Water is added and the organic layer concentrated i. vac. The residue is taken up with THF and MeOH and purified via prep. HPLC (XBridge C-18 10 μm, eluent gradient (H₂O+0.1% NH₃):ACN 61:39->41:59). The product containing fractions are combined and freeze-dried. Yield: 518 mg (1.34 mmol, 73%) Int-28a MS (ESI⁺): (M+H)⁺388; HPLC: RT=1.00 min, Method: Z011_S03

### Step 2:

To Int-28a (515 mg, 1.33 mmol) in 6.7 mL dioxane with 250 μL MeOH is added HCl in dioxane (4N, 6.6 mL, 26.6 mmol) at ambient temperature for 2.25 h. The mixture is concentrated i. vac., the residue taken up in ACN and concentrated i. vac. Yield: 520 mg (1.31 mmol, 99%) Int-28b MS (ESI⁺: (M+H)⁺288; HPLC: RT=0.79 min, Method: Z011_S03

### Step 3:

To a mixture of Int-28b (255 mg, 0.64 mmol) with 5-methyl-pyrazine-2-carboxylic acid (93 mg, 0.66 mmol) and TEA (403 μL, 2.89 mmol) in 10 mL ACN is added CIP (188 mg, 0.68 mmol) and the mixture stirred at ambient temperature for 25 min. The mixture is concentrated i. vac., the residue taken up in DCM, washed with water, dried over MgSO₄ and concentrated i. vac.

Yield: 189 mg (0.46 mmol, 72%) Int-28c

MS (ESI⁺): (M+H)⁺408; HPLC: RT=0.90 min, Method: Z011_S03

### Step 4:

To Int-28c (185 mg, 0.45 mmol) in 3.0 mL isopropanol is added K₂CO₃ (75 mg, 0.55 mmol) and the mixture stirred at 85° C. for 39 h. Then, 5 mL THF are added, the resulting mixture filtered and the filtrate purified via prep. HPLC (X-Bridge C-18 10 μm, eluent-gradient (H₂O+0.1% NH₃):ACN 61:39->41:59). The product containing fractions are combined and concentrated i. vac. The residue is further purified by chiral SFC.

Yield: 72 mg (0.19 mmol, 42%) example 28

Example 28: N-[(S)-[6-cyano-7-methyl-3-(propan-2-yl)-3H-
imidazo[4,5-b]pyridin-2-yl](cyclopropyl)methyl]-5-
methylpyrazine-2-carboxamide

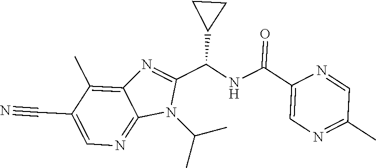 [SMILES: Cc1cnc(C(=O)N[C@H](c2nc3c(C)c(C#N)cnc3n2C(C)C)C2CC2)cn1]

| | |
|---|---|
| HPLC-MS; Method: Z011_S03; Rₜ [min]: 1.00 | MS: 390 (M + H)⁺ |
| Chiral SFC Rt Method: I_SC_20_MEOH_NH3_001 | Rt [min]: 3.09 |

¹H NMR (400 MHz, DMSO-d₆) δ ppm 0.48-0.65 (m, 4 H) 1.51-1.61 (m, 3 H) 1.63-
1.71 (m, 3 H) 1.71-1.80 (m, 1 H) 2.60 (s, 3 H) 2.72 (s, 3 H) 4.90-4.99 (m, 2 H) 8.65
(s, 1 H) 8.66 (s, 1 H) 9.04 (s, 1 H) 9.38 (d, J = 7.86 Hz, 1 H)

In analogy to example 28, the following products are obtained:

Example 36: N-[(R)-[6-cyano-7-methyl-3-(propan-2-yl)-3H-imidazo[4,5-b]pyridin-2-
yl][(2S)-oxolan-2-yl]methyl]-5-(difluoromethyl)pyrazine-2-carboxamide

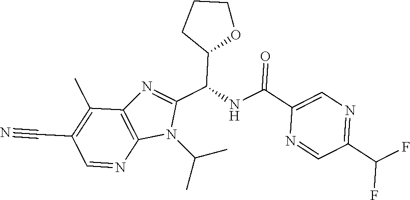 [SMILES: Cc1c(C#N)cnc2c1nc([C@@H](NC(=O)c1cnc(C(F)F)cn1)[C@@H]1CCCO1)n2C(C)C]

| | |
|---|---|
| HPLC-MS; Method: Z011_S03; Rₜ [min]: 1.05 | MS: 456 (M + H)⁺ |
| Chiral SFC Rt Method: Method I_SB_10_IPA_NH3_001 | Rt [min]: 2.93 |

¹H NMR (400 MHz, DMSO-d₆) δ ppm 1.61-1.75 (m, 6 H) 1.80-2.04 (m, 3 H) 2.10-
2.23 (m, 1 H) 2.70 (s, 3 H) 3.62-3.71 (m, 1 H) 3.80-3.88 (m, 1 H) 4.54-4.62 (m, 1
H) 5.06 (spt, J = 6.67 Hz, 1 H) 5.41 (t, J = 8.49 Hz, 1 H) 7.21 (m, 1 H) 8.66 (s, 1 H) 9.08
(s, 1 H) 9.26 (s, 1 H) 9.61 (d, J = 7.98 Hz, 1 H)

Example 40: N-[(R)-[6-cyano-7-methyl-3-(propan-2-yl)-3H-imidazo[4,5-b]pyridin-2-
yl][(2R)-oxolan-2-yl]methyl]-5-methylpyrazine-2-carboxamide

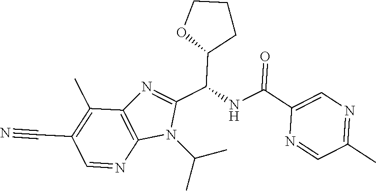 [SMILES: Cc1cnc(C(=O)N[C@H](c2nc3c(C)c(C#N)cnc3n2C(C)C)[C@H]2CCCO2)cn1]

| | |
|---|---|
| HPLC-MS; Method: Z011_S03; Rₜ [min]: 1.00 | MS: 420 (M + H)⁺ |
| Chiral SFC Rt Method: I_IA_15_ETOH_NH3_001 | Rt [min]: 2.14 |

¹H NMR (400 MHz, DMSO-d₆) δ ppm 1.52-1.61 (m, 3 H) 1.63-1.88 (m, 6 H) 2.00-
2.12 (m, 1 H) 2.61 (s, 3 H) 2.72 (s, 3 H) 3.65-3.81 (m, 2 H) 4.60 (q, J = 6.51 Hz, 1 H)
5.06 (spt, J = 6.65 Hz, 1 H) 5.59 (dd, J = 7.92, 6.65 Hz, 1 H) 8.66 (d, J = 1.14 Hz, 1 H) 8.67
(s, 1 H) 9.04-9.09 (m, 2 H)

### Example 33

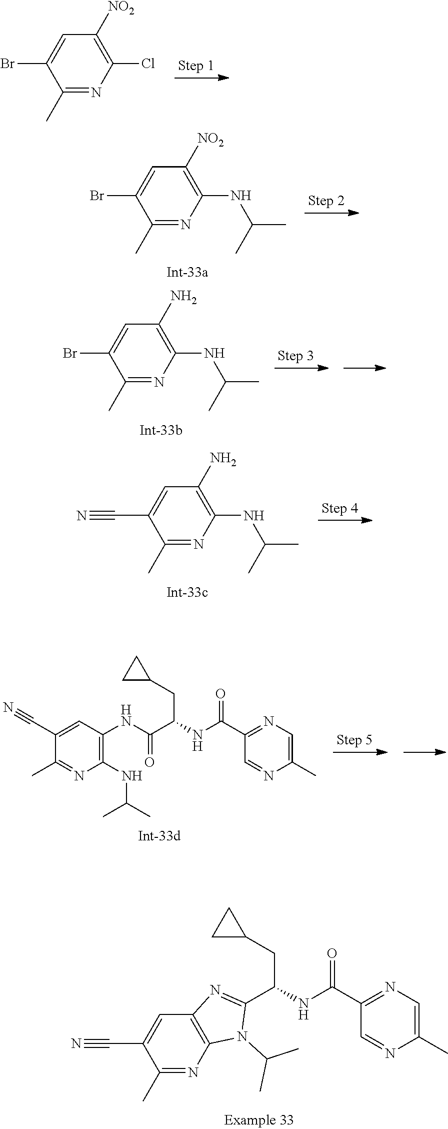 [SMILES: Cc1nc(Cl)c([N+](=O)[O-])cc1Br ; Cc1nc(NC(C)C)c([N+](=O)[O-])cc1Br ; Cc1nc(NC(C)C)c(N)cc1Br ; Cc1nc(NC(C)C)c(N)cc1C#N ; Cc1cnc(C(=O)N[C@@H](CC2CC2)C(=O)Nc2cc(C#N)c(C)nc2NC(C)C)cn1 ; Cc1cnc(C(=O)N[C@@H](CC2CC2)c2nc3cc(C#N)c(C)nc3n2C(C)C)cn1]

### Step 1:

To 3-bromo-6-chloro-2-methyl-5-nitropyridine (0.60 g, 2.39 mmol) in 5 mL DCM is added 0.56 g (9.54 mmol) isopropylamine and the mixture stirred at ambient temperature for 16 h. The mixture is concentrated i. vac., water is added, the mixture filtrated and the solid dried.

Yield: 0.66 g (2.39 mmol; quant.) Int-33a

MS (ESI⁺): (M+H)⁺274

### Step 2:

Int-33a (0.38 g, 1.39 mmol) is mixed with Raney-nickel (70 mg) in 5 mL MeOH and hydrogenated at ambient temperature for 17 h at 50 psi hydrogen pressure. Then the mixture is filtered and concentrated and dried i. vac.

Yield: 0.36 g (1.48 mmol; quant.) Int-33b

MS (ESI⁺): (M+H)⁺244/246 (Br); HPLC: RT=1.02 min, Method: Z011_S03

### Step 3:

To a mixture of Int-33b (0.36 g, 1.48 mmol) with Zn(CN)₂ (0.29 g, 2.43 mmol) in 10 mL NMP under argon is added chloro(2-dicyclohexylphosphino-2′,4′,6′-tri-isopropyl-1,1′-biphenyl)[2-(2-aminoethyl)phenyl]-palladium(II) (0.10 g, 0.135 mmol) and the mixture stirred at 110° C. for 18 h. Then water is added, the mixture stirred vigorously and filtered. The solid is dried at ambient temperature. Yield: 0.17 g (0.87 mmol; 59%) Int-33c

MS (ESI⁺): (M+H)⁺191; HPLC: RT=0.88 min, Method: Z011_S03

Step 4: Int-33c (165 mg, 0.87 mmol), Int-6f (216 mg, 0.87 mmol) and NMM (158 mg, 1.56 mmol) in 10 mL DCM are stirred at 0° C. and PPA (50% in EtOAc; 580 mg, 0.91 mmol) is added. After stirring for 1 h at 0° C., cooling is removed and the mixture stirred at ambient temperature for 16 h. The mixture is concentrated i. vac., the residue taken up with NaHCO₃ (half-conc., aq.) and extracted with DCM. The combined organic layers are concentrated i. vac.

Yield: 150 mg (0.36 mmol; 41%) Int-33d

MS (ESI⁺): (M+H)⁺422; HPLC: RT=1.01 min, Method: Z011_S03

### Step 5:

Int-33d (150 mg, 0.36 mmol) and ZnBr₂ (160 mg, 0.71 mmol) in 5 mL n-butyl acetate are stirred at 100° C. for 20 h. EtOAc and NaHCO₃ (aq., 5%) are added, filtrated and the organic layer concentrated i. vac. The residue is purified by prep. HPLC.

Yield: 8 mg (0.02 mmol; 6%) example 33

Example 33: N-[(1S)-1-[6-cyano-5-methyl-3-(propan-2-yl)-3H-imidazo[4,5-b]pyridin-
2-yl]-2-cyclopropylethyl]-5-methylpyrazine-2-carboxamide

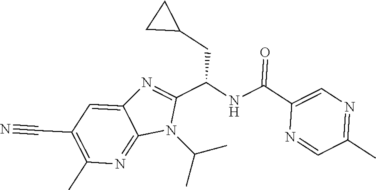 [SMILES: Cc1cnc(C(=O)N[C@@H](CC2CC2)c2nc3cc(C#N)c(C)nc3n2C(C)C)cn1]

| | |
|---|---|
| Chiral SFC Rt Method: Method I_SB_10_IPA_NH3_001 | Rt [min]: 2.93 |

¹H NMR (400 MHz, DMSO-d₆) δ ppm 0.01-0.15 (m, 2 H) 0.31-0.40 (m, 2 H) 0.70-
0.81 (m, 1 H) 1.53-1.62 (m, 3 H) 1.66-1.75 (m, 3 H) 1.93-2.15 (m, 2 H) 2.59 (s, 3
H) 2.75 (s, 3 H) 4.97 (quin, J = 6.81 Hz, 1 H) 5.60 (q, J = 7.60 Hz, 1 H) 8.53 (s, 1 H) 8.63
(d, J = 1.01 Hz, 1 H) 9.05 (d, J = 1.39 Hz, 1 H) 9.22 (d, J = 8.24 Hz, 1 H)

### Example 47

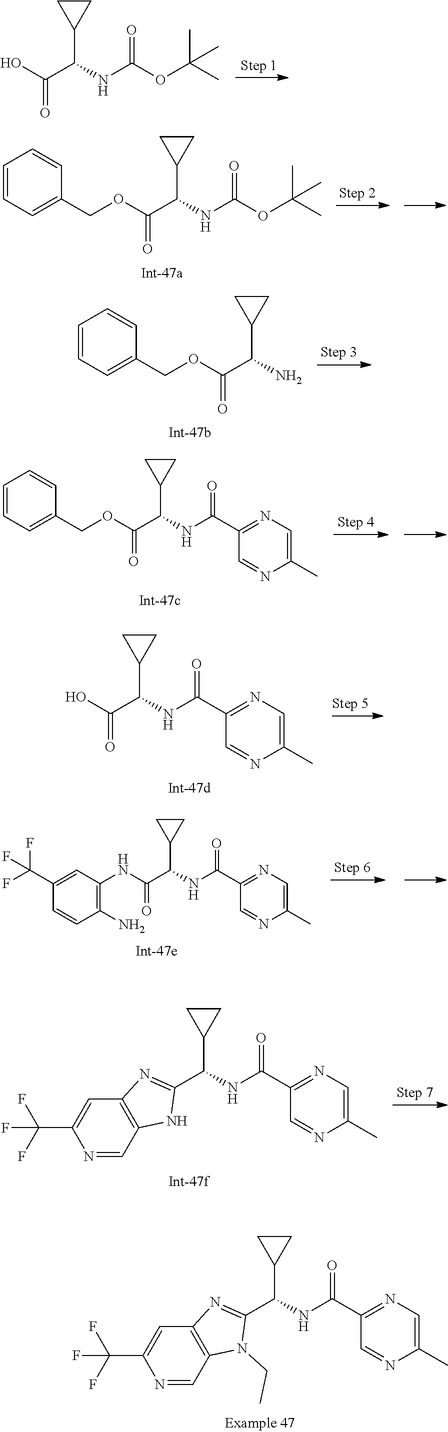

### Step 1:

NaHCO₃ (18.6 g, 221 mmol) is added to (2S)-2-Boc-amino-2-cyclopropyl-acetic acid in 160 mL DMF under stirring at ambient temperature, followed by benzylbromide (10.6 mL, 88.5 mmol). The mixture is stirred for 22 h, then filtrated and the filtrate concentrated i. vac. The residue is mixed with 500 mL water and extracted with tert.-butyl-methyl-ether. The combined organic layers are washed with water, dried over MgSO₄ and concentrated i. vac.

Yield: 25.9 g (84.8 mmol, 96%) Int-47a

MS (ESI⁺): (M+H)⁺ 306

### Step 2:

To Int-47a (25.8 g, 84.5 mmol) in 63 mL dioxane and 63 mL MeOH at 10° C. is added HCL in dioxane (4M, 127 mL, 507 mmol) under stirring. The mixture is stirred for 1.5 h at ambient temperature, then concentrated i. vac., taken up each with DCM, MeOH and diethylether and each concentrated i. vac. again. Yield: 20.4 g (84.4 mmol; quant.) Int-47b

MS (ESI⁺): (M+H)⁺206; HPLC: RT=0.87 min, Method: Z011_S03

### Step 3:

PPA (50% in EtOAc; 33 mL, 55.4 mmol) is added to a mixture of Int-47b (10.3 g, 42.6 mmol) and 2-methylpyrazine-5-carboxylic acid (6.69 g, 46.0 mmol) in 33 mL pyridine under stirring at −15° C. The mixture is stirred at 0° C. for 20 min and at ambient temperature for 1.5 h. Then, 5 mL water are added and the mixture concentrated i. vac. The residue is taken up in each 150 mL water and tert.-butyl-methyl-ether, and the aq. layer extracted with tert.-butyl-methyl-ether. The combined organic layers are washed with NaCl (aq., half-conc.), dried over MgSO₄ and concentrated i. vac. The residue is purified by chromatography on silica gel (eluent gradient: PE/EtOAc 80:20->45:55). Yield: 10.4 g (30.4 mmol; 71%) Int-47c

MS (ESI⁺): (M+H)⁺326; HPLC: RT=0.99 min, Method: Z011_S03

### Step 4:

To Int-47c (10 g, 30.7 mmol) in 76 mL dioxane is added LiOH (aq., 1N; 38.4 mL, 38.4 mmol) and the mixture stirred at ambient temperature for 1 h. Then the mixture is adjusted to pH3 by addition of HCl (aq., 4N, 9.6 mL, 38.4 mmol) and freezedried. The residue is taken up in water, filtrated, the solid washed with water and dried i. vac.

Yield: 5.82 g (24.7 mmol; 80%) Int-47d

MS (ESI⁺): (M+H)⁺236; HPLC: RT=0.73 min, Method: Z018_S04

### Step 5:

NMM (752 μL, 6.84 mmol) and PPA (50% in EtOAc; 1.4 mL, 2.35 mmol) are added to a mixture of 4,5-diamino-2-trifluoromethyl-pyridine (416 mg, 2.35 mmol) and Int-47d (460 mg, 1.96 mmol) in 40 mL DCM under stirring at −10° C. The mixture is stirred at 0° C. for 3 h. Then, DCM is added and the mixture extracted with NaHCO₃ (aq., 5%). The aq. layer is extracted with DCM and the combined organic layers dried over Na₂SO₄ and concentrated i. vac.

Yield: 579 mg (1.47 mmol; 75%) Int-47e

MS (ESI⁺): (M+H)⁺395; HPLC: RT=0.79 min, Method: Z011_S03

### Step 6:

Int-47e (579 mg, 1.47 mmol) is stirred in 5 mL AcOH at 85° C. for 4d. The mixture is concentrated i. vac., the residue taken up in MeOH, filtrated and the filtrate purified by HPLC (XBridge C18, 10 μm, eluent: (H₂O+0.15% NH₃):ACN 91:9). The product containing fractions are combined and concentrated i. vac.

Yield: 224 mg (0.60 mmol; 40%) Int-47f

MS (ESI⁺): (M+H)⁺377; HPLC: RT=0.69 min, Method: Z011_S03

### Step 7:

To Int-47f (223 mg, 0.59 mmol) in 5.0 mL DMF is added Cs₂CO₃ (290 mg, 0.89 mmol) and iodoethane (57 μL, 0.71 mmol) and the mixture stirred at 60° C. for 4.5 h. Then, more iodoethane (20 μL, 0.21 mmol) is added and the mixture stirred at 60° C. for 1.5 h. ACN is added, filtrated and concentrated i. vac. The residue is purified via prep. HPLC (XBridge C-18 10 μm at 60° C., eluent (H₂O+0.1% NH₃):MeOH 61:39->41:59). The product containing fractions are combined and concentrated i. vac. The residue is further purified by chiral SFC.

Yield: 10 mg (0.025 mmol, 4.2%)

Example 47: N-[(S)-cyclopropyl[3-ethyl-6-(trifluoromethyl)-3H-imidazo[4,5-c]pyridin-
2-yl]methyl]-5-methylpyrazine-2-carboxamide

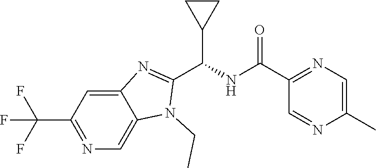 [SMILES: CCn1c([C@@H](NC(=O)c2cnc(C)cn2)C2CC2)nc2cc(C(F)(F)F)ncc21]

| | |
|---|---|
| | MS: 405 (M + H)⁺ |
| Chiral SFC Rt Method: I_SC_20_IPA_NH3_001 | Rt [min]: 2.07 |

¹H NMR (400 MHz, DMSO-d₆) δ ppm 0.43-0.65 (m, 4 H) 1.31-1.43 (m, 3 H) 1.72-
1.83 (m, 1 H) 2.60 (s, 3 H) 4.41-4.62 (m, 2 H) 4.99 (m, 1 H) 8.18 (s, 1 H) 8.65 (d,
J = 1.01 Hz, 1 H) 9.04 (d, J = 1.39 Hz, 1 H) 9.14 (s, 1 H) 9.34 (d, J = 7.98 Hz, 1 H)

In analogy to example 47, the following products are obtained:

Example 48: N-[(S)-cyclopropyl[3-ethyl-6-(trifluoromethyl)-3H-imidazo[4,5-c]pyridin-
2-yl]methyl]-5-(difluoromethyl)pyrazine-2-carboxamide

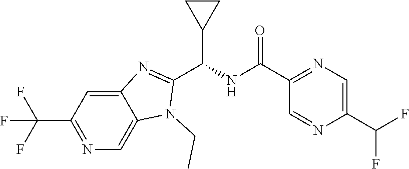 [SMILES: CCn1c([C@@H](NC(=O)c2cnc(C(F)F)cn2)C2CC2)nc2cc(C(F)(F)F)ncc21]

| | |
|---|---|
| | MS: 441 (M + H)⁺ |
| Chiral SFC Rt Method: I_SC_10_IPA_NH3_001 | Rt [min]: 2.23 |

¹H NMR (400 MHz, DMSO-d₆) δ ppm 0.00 (s, 1 H) 0.44-0.68 (m, 4 H) 1.38 (t, J = 7.16
Hz, 3 H) 1.76-1.86 (m, 1 H) 4.43-4.62 (m, 2 H) 5.00 (m, 1 H) 7.22 (m, 1 H) 8.18 (s, 1
H) 9.06 (s, 1 H) 9.16 (s, 1 H) 9.26-9.30 (m, 1 H) 9.62 (d, J = 7.73 Hz, 1 H)

Example 49: N-[(S)-cyclopropyl[1-ethyl-6-(trifluoromethyl)-1H-imidazo[4,5-c]pyridin-
2-yl]methyl]-5-(difluoromethyl)pyrazine-2-carboxamide

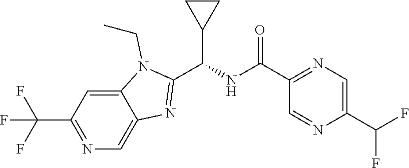 [SMILES: CCn1c([C@@H](NC(=O)c2cnc(C(F)F)cn2)C2CC2)nc2cnc(C(F)(F)F)cc21]

| | |
|---|---|
| | MS: 441 (M + H)⁺ |
| Chiral SFC Rt Method: I_IG_25_IPA_NH3_001 | Rt [min]: 2.24 |

¹H NMR (400 MHz, DMSO-d₆) δ ppm 0.43-0.67 (m, 4 H) 1.32 (t, J = 7.16 Hz, 3 H)
1.77-1.86 (m, 1 H) 4.39-4.57 (m, 2 H) 5.02 (m, 1 H) 7.21 (m, 1 H) 8.28-8.31 (m, 1
H) 9.06 (s, 1 H) 9.08 (s, 1 H) 9.27-9.30 (m, 1 H) 9.60 (d, J = 7.86 Hz, 1 H)

1-20. (canceled)

21. A compound of formula I 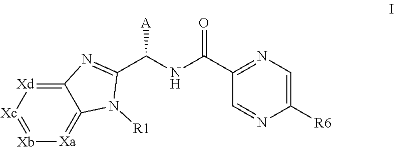
wherein
A is selected from the group consisting of C₁-C₆-alkyl, C₃-C₆-cycloalkyl, C₃-C₅-cycloalkyl-C₁-C₂-alkyl-, C₁-C₃-alkyl-O—C₁-C₃-alkyl-, 4-6-membered heterocycloalkyl-, and 4-6-membered heterocycloalkyl-C₁-C₃-alkyl-, wherein each of said C₁-C₆-alkyl, C₃-C₆-cycloalkyl, C₃-C₅-cycloalkyl-C₁-C₂-alkyl-, C₁-C₃-alkyl-O—C₁-C₃-alkyl-, 4-6-membered heterocycloalkyl-, and 4-6-membered heterocycloalkyl-C₁-C₃-alkyl- A groups is optionally substituted with 1-4 substituents selected from the group consisting of C₁-C₄-alkyl, C₁-C₄-alkoxy, hydroxy, and fluoro;
Xa is N or C—R²
Xb is N or C—R³;
Xc is N or C—R⁴;
Xd is C—R⁵;
provided that one of Xa, Xb and Xc is N;
R¹ is selected from the group consisting of C₁-C₇-alkyl, C₁-C₃-alkyl-O—C₁-C₃-alkyl-, C₃-C₇-cycloalkyl, 4-6-membered heterocycloalkyl, C₃-C₇-cycloalkyl-C₁-C₃-alkyl-, 4-6-membered heterocycloalkylmethyl-, and C₅-C₆-heterocycloalkylethyl-, wherein each of said C₁-C₇-alkyl, C₁-C₃-alkyl-O—C₁-C₃-alkyl-, C₃-C₇-cycloalkyl, 4-6-membered heterocycloalkyl, C₃-C₇-cycloalkyl-C₁-C₃-alkyl-, and 4-6-membered heterocycloalkylmethyl-R¹ groups is optionally substituted with 1-4 substituents selected from the group consisting of C₁-C₄-alkyl, C₁-C₄-alkoxy, C₃-C₇-cycloalkoxy, hydroxy, and fluoro;
R², R³, R⁴ and R⁵ are each independently of each other is selected from the group consisting of hydrogen, halogen, cyano, C₁-C₄-alkyl, C₁-C₃-alkyl-O—C₁-C₃-alkyl-, C₃-C₆-cycloalkyl, 4-6-membered C₄-C₆-heterocycloalkyl, C₁-C₄-alkoxy-, and C₃-C₆-cycloalkoxy-, wherein each of said C₁-C₄-alkyl, C₁-C₃-alkyl-O—C₁-C₃-alkyl-, C₃-C₆-cycloalkyl, 4-6-membered C₄-C₆-heterocycloalkyl, C₁-C₄-alkoxy-, and C₃-C₆-cycloalkoxy-R², R³, R⁴ and R⁵ groups is optionally substituted with 1-4 substituents chosen from C₁-C₄-alkyl, C₁-C₄-alkoxy, hydroxy, and fluoro;
R⁶ is selected from the group consisting of halogen and C₁-C₃-alkyl optionally substituted with 2-3 fluorine atoms;
or a physiologically acceptable salt thereof.

22. The compound according to claim 21 of formula Ia, Ib or Ic 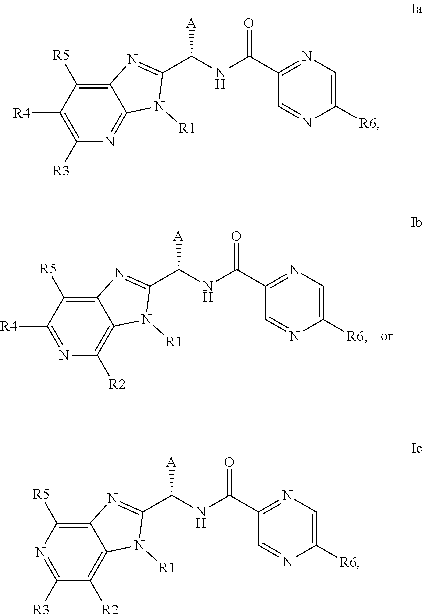
or a physiologically acceptable salt thereof.

23. The compound according to claim 21, wherein
A is selected from the group consisting of C₁-C₃-alkyl, C₃-C₆-cycloalkyl, C₃-C₅-cycloalkylmethyl-, tetrahydrofuranyl-, tetrahydropyranyl-, 1,4-dioxanyl, tetrahydrofuranylmethyl-, tetrahydropyranylmethyl-, 1,4-dioxan-ylmethyl-, and C₁-C₂-alkyl-O—C₁-C₃-alkyl-, wherein each of said C₁-C₃-alkyl, C₃-C₆-cycloalkyl, C₃-C₅-cycloalkylmethyl-, tetrahydrofuranyl-, tetrahydropyranyl-, 1,4-dioxanyl, tetrahydrofuranylmethyl-, tetrahydropyranylmethyl-, 1,4-dioxan-ylmethyl-, C₁-C₂-alkyl-O—C₁-C₃-alkyl- A groups is optionally substituted with 1-4 substituents chosen from methyl, methoxy, hydroxy, and fluoro,
or a physiologically acceptable salt thereof.

24. The compound according to claim 21, wherein
R¹ is selected from the group consisting of C₁-C₃-alkyl, C₁-C₂-alkyl-O—C₁-C₃-alkyl-, C₃-C₄-cycloalkyl, C₄-C₅-heterocycloalkyl-, C₃-C₄-cycloalkyl-O—C₁-C₃-alkyl-, wherein each of said C₁-C₃-alkyl, C₁-C₂-alkyl-O—C₁-C₃-alkyl-, C₃-C₄-cycloalkyl, C₄-C₅-heterocycloalkyl-, C₃-C₄-cycloalkyl-O—C₁-C₃-alkyl- R¹ groups is optionally substituted with 1-4 substituents is selected from the group consisting of C₁-C₄-alkyl, C₁-C₄-alkoxy, C₃-C₄-cycloalkoxy, hydroxy and fluoro,
or a physiologically acceptable salt thereof.

25. The compound according to claim 21, wherein
R², R³, R⁴ and R⁵ are independently of each other selected from the group consisting of hydrogen, fluoro, chloro, bromo, cyano, methyl, cyclopropyl and methoxy, wherein said methyl, cyclopropyl and methoxy groups are optionally substituted with 2-3 fluorine atoms,
or a physiologically acceptable salt thereof.

26. The compound according to claim 21, wherein
R⁶ is C₁-C₃-alkyl optionally substituted with 2-3 fluorine atoms,
or a physiologically acceptable salt thereof.

27. The compound according to claim 21, wherein
A is selected from the group consisting of: 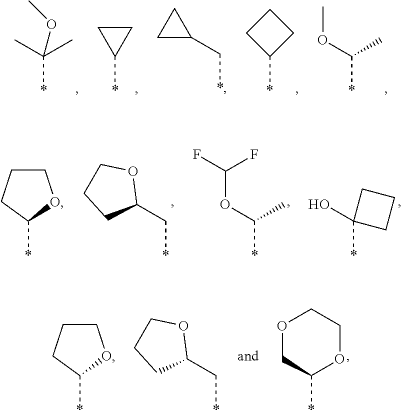
or a physiologically acceptable salt thereof

28. The compound according to claim 21, wherein
R¹ is selected from the group consisting of ethyl, —CH₂—CHF₂, and iso-propyl,
or a physiologically acceptable salt thereof.

29. The compound according to claim 21, wherein
R² is hydrogen;
R³ is selected from the group consisting of hydrogen, methyl and trifluromethyl;
R⁴ is selected from the group consisting of hydrogen, fluoro, chloro, bromo, cyano, methyl and trifluromethyl; and
R⁵ is selected from the group consisting of hydrogen, methyl and methoxy,
or a physiologically acceptable salt thereof.

30. The compound according to claim 21, wherein
R⁶ is selected from the group consisting of methyl, trifluromethyl and —CF₂H,
or a physiologically acceptable salt thereof.

31. The compound according to claim 21 selected from the group consisting of: 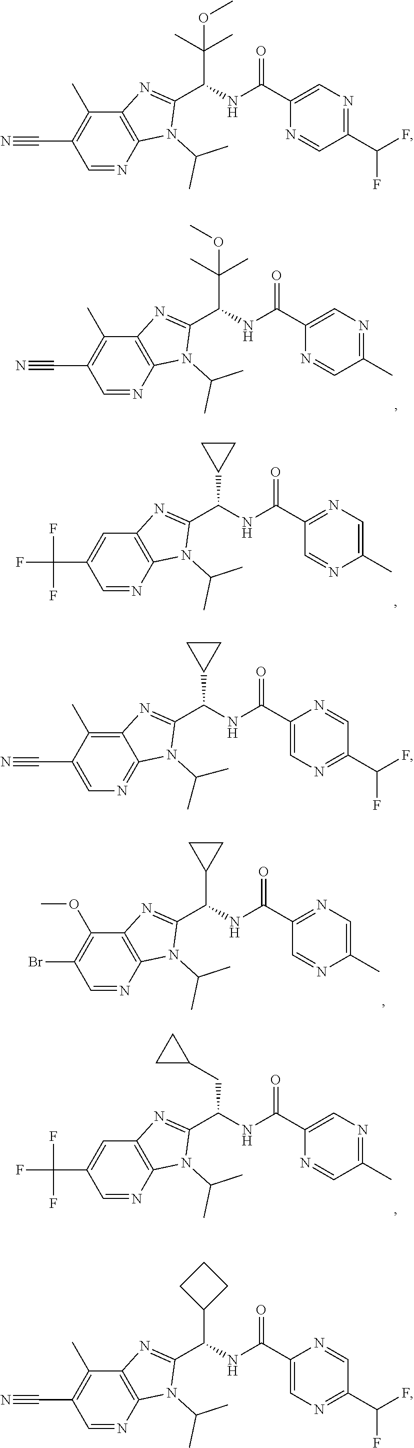 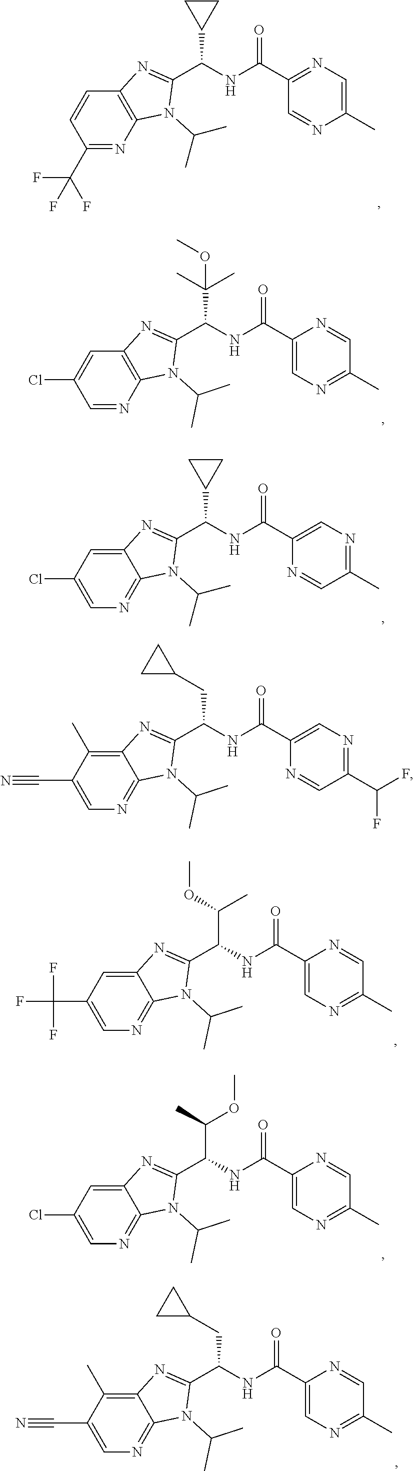 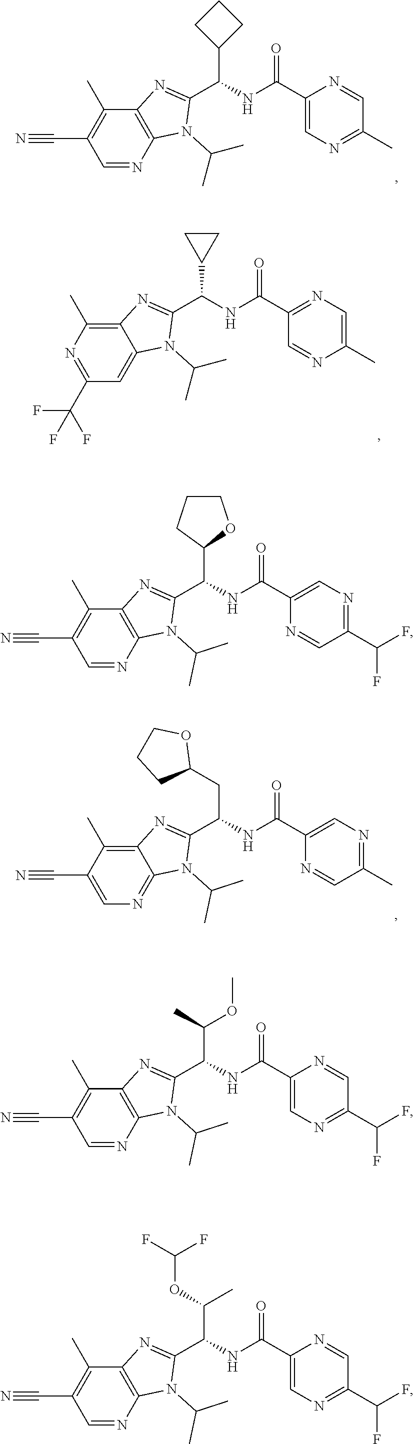 [SMILES: Cc1cnc(C(=O)N[C@H](c2nc3c(C)c(C#N)cnc3n2C(C)C)C2CCC2)cn1 ; Cc1cnc(C(=O)N[C@H](c2nc3c(C)nc(C(F)(F)F)cc3n2C(C)C)C2CC2)cn1 ; Cc1c(C#N)cnc2c1nc([C@@H](NC(=O)c1cnc(C(F)F)cn1)[C@H]1CCCO1)n2C(C)C ; Cc1cnc(C(=O)N[C@@H](C[C@H]2CCCO2)c2nc3c(C)c(C#N)cnc3n2C(C)C)cn1 ; CO[C@H](C)[C@H](NC(=O)c1cnc(C(F)F)cn1)c1nc2c(C)c(C#N)cnc2n1C(C)C ; Cc1c(C#N)cnc2c1nc([C@@H](NC(=O)c1cnc(C(F)F)cn1)[C@@H](C)OC(F)F)n2C(C)C] 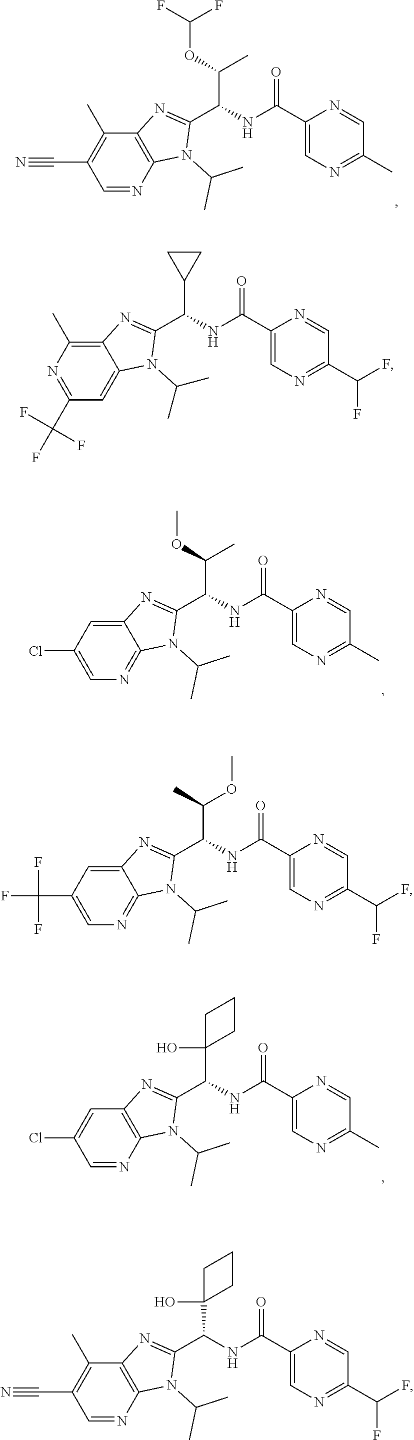 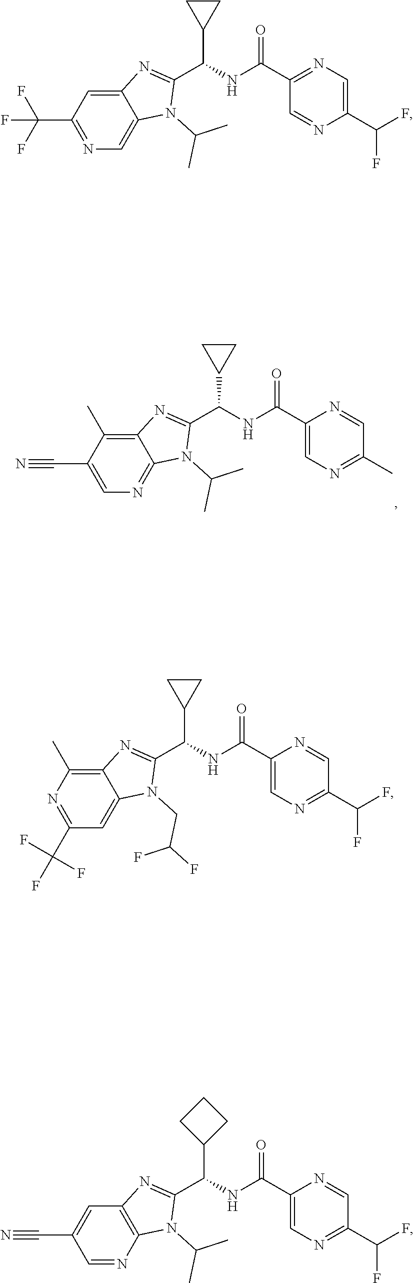 [SMILES: CC(C)n1c([C@@H](NC(=O)c2cnc(C(F)F)cn2)C2CC2)nc2cc(C(F)(F)F)ncc21 ; Cc1cnc(C(=O)N[C@H](c2nc3c(C)c(C#N)cnc3n2C(C)C)C2CC2)cn1 ; Cc1nc(C(F)(F)F)cc2c1nc([C@@H](NC(=O)c1cnc(C(F)F)cn1)C1CC1)n2CC(F)F ; CC(C)n1c([C@@H](NC(=O)c2cnc(C(F)F)cn2)C2CCC2)nc2cc(C#N)cnc21] 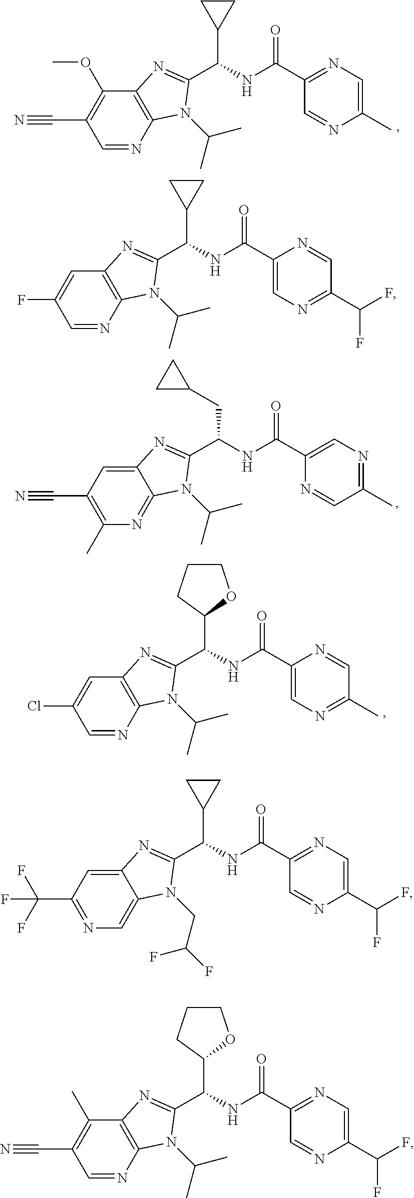 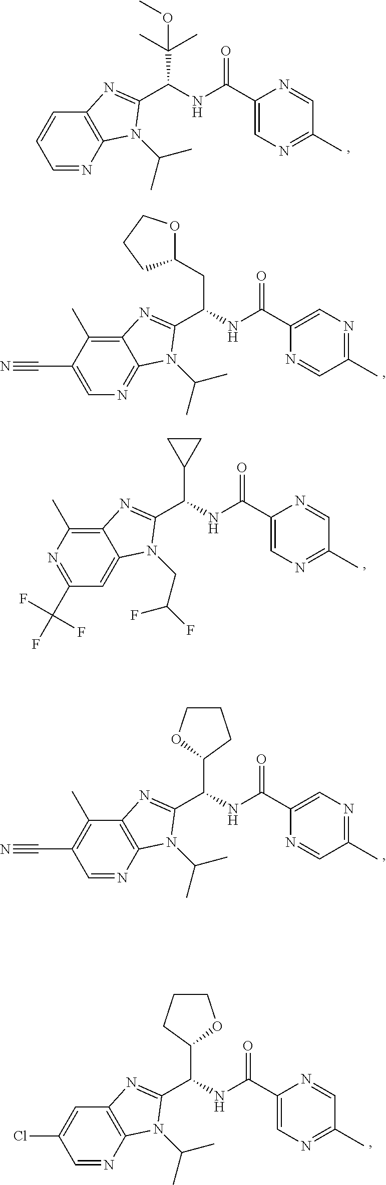 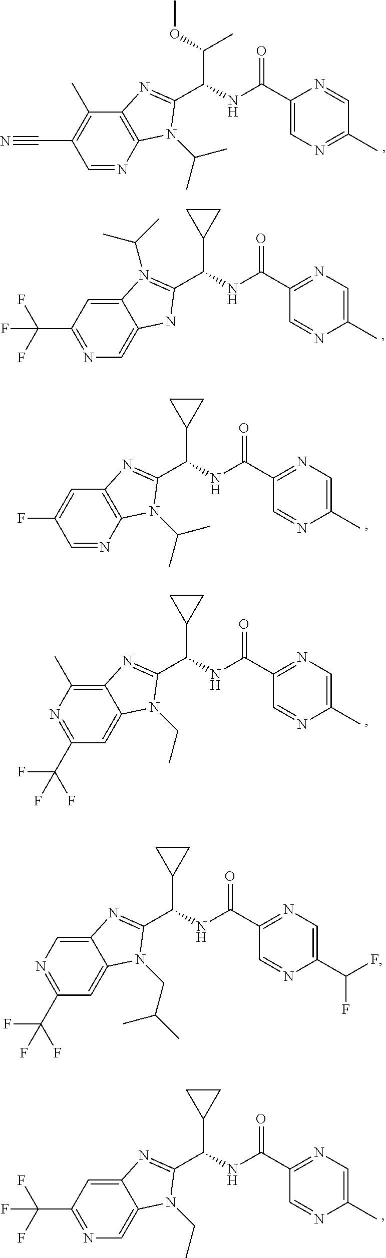 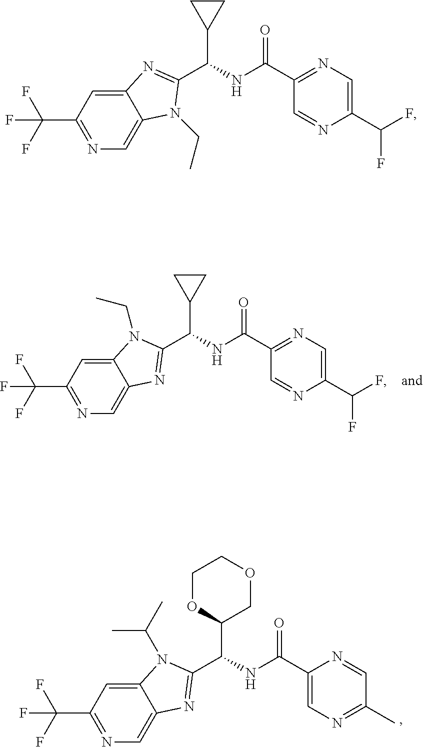 [SMILES: CCn1c([C@@H](NC(=O)c2cnc(C(F)F)cn2)C2CC2)nc2cc(C(F)(F)F)ncc21 ; CCn1c([C@@H](NC(=O)c2cnc(C(F)F)cn2)C2CC2)nc2cnc(C(F)(F)F)cc21 ; "and" ; Cc1cnc(C(=O)N[C@H](c2nc3cnc(C(F)(F)F)cc3n2C(C)C)[C@@H]2COCCO2)cn1]
or a physiologically acceptable salt thereof.

32. A physiologically acceptable salt of the compound according to claim 21.

33. A pharmaceutical composition comprising the compound according to claim 21, or a physiologically acceptable salt thereof.

34. A method of treating or preventing a condition or disorder that can be mediated by inhibiting mGluR4, comprising administering to a patient in need therefor a therapeutically effective amount of the compound of claim 21, or a physiologically acceptable salt thereof, wherein the mGluR4 mediated disease or disorder psychiatric is:
a neurological, neurodegenerative, non-neuronal disease or disorder;
a metabolic disease or disorder; or
cancer or a related disease or disorder.

35. The method of claim 34, wherein the mGluR4 mediated disease or disorder is selected from the group consisting of:
impulse control deficits or maladaptive impulsivity;
substance use disorders;
personality disorders selected from the group consisting of borderline personality disorder, antisocial personality disorder, and conduct disorder;
eating disorder;
attention deficit hyperactivity disorder;
bipolar disorder;
post-traumatic stress disorder;
tic disorders;
movement disorders;
cognitive dysfunction in psychiatric or neurological disorder;
cognitive impairments associated with schizophrenia;
Alzheimer's disease;
overweight;
obesity; and
cancer and related disorders associated with maladaptive tumor genesis like osteosarcoma.
